SEQUENCE LISTING (65 sequences)

SEQ ID NO: 1 - DNA, 20 bp, Artificial Sequence
  Description of Artificial Sequence Synthetic oligonucleotide
cggagttactttgaacaata

SEQ ID NO: 2 - DNA, 20 bp, Artificial Sequence
  Description of Artificial Sequence Synthetic oligonucleotide
acaatatggtactgtgaagg

SEQ ID NO: 3 - DNA, 20 bp, Artificial Sequence
  Description of Artificial Sequence Synthetic oligonucleotide
tgaacaatatggtactgtga

SEQ ID NO: 4 - DNA, 25 bp, Artificial Sequence
  Description of Artificial Sequence Synthetic oligonucleotide
caccgcggagttactttgaacaata

SEQ ID NO: 5 - DNA, 25 bp, Artificial Sequence
  Description of Artificial Sequence Synthetic oligonucleotide
aaactattgttcaaagtaactccgc

SEQ ID NO: 6 - DNA, 25 bp, Artificial Sequence
  Description of Artificial Sequence Synthetic oligonucleotide
caccgacaatatggtactgtgaagg

SEQ ID NO: 7 - DNA, 25 bp, Artificial Sequence
  Description of Artificial Sequence Synthetic oligonucleotide
aaacccttcacagtaccatattgtc

SEQ ID NO: 8 - DNA, 25 bp, Artificial Sequence
  Description of Artificial Sequence Synthetic oligonucleotide
caccgtgaacaatatggtactgtga

SEQ ID NO: 9 - DNA, 25 bp, Artificial Sequence
  Description of Artificial Sequence Synthetic oligonucleotide
aaactcacagtaccatattgttcac

SEQ ID NO: 10 - DNA, 9292 bp, Artificial Sequence
  Description of Artificial Sequence Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcggagttactttgaacaatagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - DNA, 1458 bp, Artificial Sequence
  Description of Artificial Sequence Synthetic polynucleotide
gcagaggaggtgtaagagaagagatggagcaaactgtctctgctttttaggggtagtctggtgttaatttggttcactcatgttgaaatcatacacgtggaaagctgagttctagttctggcgttacttaattgctggttgaatcaggccacatggacagtttggttcttgtaattttcttagtcccttaccggatgtccaatttgaatgaacattgatcacttgcaggtgcagtttaaataactctgtgttaaacttctaatttgttacaatcacgagagcccatttttcaatgtaaattattcttaggtttcaaagtatcagtaacctcaactaaatcaaaagatctgcctatctgaaatagggataatgctacaccaggtgagctgcaaggaaaaggttattaatgttttgagatgtcttaatacagacaaatgagcacaaataaggtggtcaaagtagttgttttttttacaagccagataaagaatgacatgtacatagaaccattcattcagttgagaagatgtggaaccaaagtttcatccatctgaggtgtatttcagttcttgcaaatatctttgtgtaatgtttgaagtgtgttttagagtatggaacacgtcttggtgtcatcagcaacaagaaatggaattgtgtggtctctgtgaacaaatgattccctaaataaacagtaatccagaatccactttcctctgacctgaactgagtgagaaactttgaggctgtgagttacgttcaagtttaaagggtgcacgtggaatgtgggtgtgcgaagcacatcaccgctgtagttattccattacaccatgtagatatgtgcagtgcactcttaagatcctgcttcggtgtgtgccactcagtgacaagatcagtcgttcatatttctcttgtagttaaattgactaaaactttttttctgagtgcacataaagtgaatattctaccaagacggtgtcatctaccttacagctaaattgtagtataactgtaccattgttccaaggaatttcataagctttactcacttcttgagcattacaggcttttgatcaggaaaatggagttcattcattggataatcaattcacagtgtagaacttaagaatttcttgcttgcatctaaaaggaattgtgtaaaaatttgcgatgaataatttcggggttctcattgtaagttgggtagtagcaacaatggttgtgaagcttccagtcaggcaaggctgcttagtgtagcctagcttgagtcttgatctcaaagaagagtaggttcaaacttgtccatttgagtgttccatttgcagtaagtacttcctttaagtaattggaaagatgctattgatacttacggtctcaaactgtatcaataggaagatggaggcctacctagtagtgtattacaatgtgactgaaatatttctgtttaaccttttcagtctg

SEQ ID NO: 12 - DNA, 707 bp, Artificial Sequence
  Description of Artificial Sequence Synthetic polynucleotide
tgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 13 - DNA, 246 bp, Artificial Sequence
  Description of Artificial Sequence Synthetic polynucleotide
cgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaact

SEQ ID NO: 14 - DNA, 1534 bp, Artificial Sequence
  Description of Artificial Sequence Synthetic polynucleotide
actgacagaactggtgtttccaaagggtgagcagaatgtcattagttactgcttttgtagatgtaattctaacataaatgatgtctgttgttaagtagttggtcacttaccatgcttaagcctttcaaactggggtgaattaaagtgaaacatgtaagatcatatagatttaagatcagtcaagttttacaattgagaactggacagattttatggtgtacctgttcagggaaaatagtgttaatgtcactcaaccagtgggagcaaagcataaaacgtagtggatgcttgtggggactgttttacaggctgaaattttgactttctgatggccatagcaattaagcagccatcagtgtagtaccactaatgtaattgagacagggagtagactttcattggggcagttggactgcagtcttttgttgctcaggggtaagttagaggcaatcaaactgtttcaggtggtgagtgaaacttaagggatggtagaaaattagagacattcccattggatatgtagaaagtactctgatctgtagtgaagaacttaagtgaagatgcctaggactctgcccagttgagttcagaggaagctctcccagctttgaaattagacttgctttgcgaggaagacttcacctctaaagatgcaccaattgttttctctgagcaggttccaaaaagtagcattttttttttaatagacacatatagtaatgagctgaaaatactgagcttaatgtctcttgcctggtctttgtggtgaattctaatgtgtgattagcaagcatatgttctgattattgataaattgctgtatgtcaatcagtggaatactctactgcagttctgagaattgtctccaatattaaggcttaaataaacaagaggtagtgagataaattgaaaacctcttttgggatcgcttcctccaatagtgtaattattcctgtagttcctcctttcattcaaacctctgcaggaagtacagaatttagtacatactaattgaaggagcttttggctttctgatgctactaatattaacagtagtactcacttgagtaatttaaatgagagaatattgaatgtggcatttaattcctttcatttggcccagtgtgctgtcagtcagcagcaaatgtactttcatgctgaattatatattaatgtcctgttaatatcagttaatgttctttttactgttttagttttttttaaaaaaaaaactaacagctgtcaaaaaatgaaaatgtagtatttgaataatattttttttcttttcaggtatggatttgtttcattcctggacaatgtggatgttcaaaagatagtagaagtaagctctttatgtcttaagttgtcagaagaaccttctgtatgaaggttgtaggtgtggttaggggataccagtcccaactgagaaaataaaaaagactagaagtgccccaaagtaaacttgcttaaatattgttgtgatttaacccagcagattgtgaagtaccatgtagtattttcctcactgcactcc

SEQ ID NO: 15 - DNA, 463 bp, Artificial Sequence
  Description of Artificial Sequence Synthetic polynucleotide
caaatgcggaagcccagtgtggaactatctcagaggataatacccattcgtcaacaacctgccaaggatatgttttaccagaaggaaaaatcatgccaaatacagtctttgttggtggaattgatataagggtatttatgtactttcaatggttttaaactacatatgacacgctgtagtgggaaagaaataagaattttaacttctggagggcttttttttaattgggtctttactgatcttgaaataatgcattatggtaagagaactttgaaacaaacaaaagagattttcctggaatattaggagatgtgtttaaaaatggtacttgttgctttaaaacaattgtaaccgtttactgtgtctgtgaagtagttcaagacttggtttcttttagatgaatgaagcagaaattcggagttactttgaacaatatggtactgtgaaggaggtgaaaataatc

SEQ ID NO: 16 - DNA, 22 bp, Artificial Sequence
  Description of Artificial Sequence Synthetic oligonucleotide
tgatagcacagtaagctgattc

SEQ ID NO: 17 - DNA, 20 bp, Artificial Sequence
  Description of Artificial Sequence Synthetic oligonucleotide
cgtacatgaactgaggggac

SEQ ID NO: 18 - DNA, 22 bp, Artificial Sequence
  Description of Artificial Sequence Synthetic oligonucleotide
ttgtttattgcagcttataatg

SEQ ID NO: 19 - DNA, 20 bp, Artificial Sequence
  Description of Artificial Sequence Synthetic oligonucleotide
accccgttacccatttttcc

SEQ ID NO: 20 - DNA, 20 bp, Artificial Sequence
  Description of Artificial Sequence Synthetic oligonucleotide
ccttttcagtctgcaaatgc

SEQ ID NO: 21 - DNA, 22 bp, Artificial Sequence
  Description of Artificial Sequence Synthetic oligonucleotide
attttccctgaacagatacacc

SEQ ID NO: 22 - DNA, 712 bp, Artificial Sequence
  Description of Artificial Sequence Synthetic polynucleotide
ccttttcagtctgcaaatgcggaagcccagtgtggaagtatctcagaggataatacccattcgtcaacaacctgccaaggatatgttttaccagaaggaaaaatcatgccaaatacagtctttgttggtggaattgatataagggtatttatgtactttcaatggttttaaactacatatgacacgctgtagtgggaaagaaataagaattttaacttctggagggcttttttttaattgggtctttaccgatcttgaaataatgcattatggtaagagaactttgaaacaaacaagagagattttcctggaatattaggagatgtgtttaaaaatggtacttgttgctttaaaacaattgtaaccgtttactgtgtctgtgaagtagttcaagacttggtttcttttagatgaatgaagcagaaattcggagttactttgaacaatatggtactgtgaaggaggtgaaaataatcactgacagaactggtgtttccaaagggtgagcagaatgtcattagttactgcttttgtagatgtaattctaacataaatgatgtctgttgttaagtagttggtcacttaccatgcttaagcctttcaaattgagatgaattaaagtgaaacatgtaaaactcgactgatcttaaatctgtataatcttacaattgagaactggacatattttatggtgtatctgttcagggaaaat

SEQ ID NO: 23 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 24 - DNA, 711 bp, Artificial Sequence
  Description of Artificial Sequence Synthetic polynucleotide
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 25 - DNA, 26 bp, Artificial Sequence
  Description of Artificial Sequence Synthetic oligonucleotide
aaccttttcagtctgcaaatgcggaa

SEQ ID NO: 26 - protein, 5 aa, Artificial Sequence
  Description of Artificial Sequence Synthetic peptide
SANAE

SEQ ID NO: 27 - DNA, 75 bp, Artificial Sequence
  Description of Artificial Sequence Synthetic oligonucleotide
gaaattcggagttactttgaacaatatggtactgtgaaggaggtgaaaataatcactgacagaactggtgtttcc

SEQ ID NO: 28 - protein, 25 aa, Artificial Sequence
  Description of Artificial Sequence Synthetic peptide
EIRSYFEQYGTVKEVKIITDRTGVS

SEQ ID NO: 29 - DNA, 35 bp, Artificial Sequence
  Description of Artificial Sequence Synthetic oligonucleotide
aaccttttcagtctgtgagcaagggcgaggaggat

SEQ ID NO: 30 - protein, 8 aa, Artificial Sequence
  Description of Artificial Sequence Synthetic peptide
SVSKGEED

SEQ ID NO: 31 - DNA, 21 bp, Artificial Sequence
  Description of Artificial Sequence Synthetic oligonucleotide
actgacagaactggtgtttcc

SEQ ID NO: 32 - protein, 7 aa, Artificial Sequence
  Description of Artificial Sequence Synthetic peptide
TDRTGVS

SEQ ID NO: 33 - DNA, 9292 bp, Artificial Sequence
  Description of Artificial Sequence Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaatatggtactgtgaagggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 34 - DNA, 9292 bp, Artificial Sequence
  Description of Artificial Sequence Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgaacaatatggtactgtgagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 35 - DNA, 21 bp, Artificial Sequence
  Description of Artificial Sequence Synthetic oligonucleotide
tctgtgagcaagggcgaggag

SEQ ID NO: 36 - protein, 7 aa, Artificial Sequence
  Description of Artificial Sequence Synthetic peptide
SVSKGEE

SEQ ID NO: 37 - DNA, 6967 bp, Artificial Sequence
  Description of Artificial Sequence Synthetic polynucleotide
ggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttccatggcagctgagaatattgtaggagatcttctagaaagatgcagaggaggtgtaagagaagagatggagcaaactgtctctgctttttaggggtagtctggtgttaatttggttcactcatgttgaaatcatacacgtggaaagctgagttctagttctggcgttacttaattgctggttgaatcaggccacatggacagtttggttcttgtaattttcttagtcccttaccggatgtccaatttgaatgaacattgatcacttgcaggtgcagtttaaataactctgtgttaaacttctaatttgttacaatcacgagagcccatttttcaatgtaaattattcttaggtttcaaagtatcagtaacctcaactaaatcaaaagatctgcctatctgaaatagggataatgctacaccaggtgagctgcaaggaaaaggttattaatgttttgagatgtcttaatacagacaaatgagcacaaataaggtggtcaaagtagttgttttttttacaagccagataaagaatgacatgtacatagaaccattcattcagttgagaagatgtggaaccaaagtttcatccatctgaggtgtatttcagttcttgcaaatatctttgtgtaatgtttgaagtgtgttttagagtatggaacacgtcttggtgtcatcagcaacaagaaatggaattgtgtggtctctgtgaacaaatgattccctaaataaacagtaatccagaatccactttcctctgacctgaactgagtgagaaactttgaggctgtgagttacgttcaagtttaaagggtgcacgtggaatgtgggtgtgcgaagcacatcaccgctgtagttattccattacaccatgtagatatgtgcagtgcactcttaagatcctgcttcggtgtgtgccactcagtgacaagatcagtcgttcatatttctcttgtagttaaattgactaaaactttttttctgagtgcacataaagtgaatattctaccaagacggtgtcatctaccttacagctaaattgtagtataactgtaccattgttccaaggaatttcataagctttactcacttcttgagcattacaggcttttgatcaggaaaatggagttcattcattggataatcaattcacagtgtagaacttaagaatttcttgcttgcatctaaaaggaattgtgtaaaaatttgcgatgaataatttcggggttctcattgtaagttgggtagtagcaacaatggttgtgaagcttccagtcaggcaaggctgcttagtgtagcctagcttgagtcttgatctcaaagaagagtaggttcaaacttgtccatttgagtgttccatttgcagtaagtacttcctttaagtaattggaaagatgctattgatacttacggtctcaaactgtatcaataggaagatggaggcctacctagtagtgtattacaatgtgactgaaatatttctgtttaaccttttcagtctgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatctactagtagatccatgcatatcactgacagaactggtgtttccaaagggtgagcagaatgtcattagttactgcttttgtagatgtaattctaacataaatgatgtctgttgttaagtagttggtcacttaccatgcttaagcctttcaaactggggtgaattaaagtgaaacatgtaagatcatatagatttaagatcagtcaagttttacaattgagaactggacagattttatggtgtacctgttcagggaaaatagtgttaatgtcactcaaccagtgggagcaaagcataaaacgtagtggatgcttgtggggactgttttacaggctgaaattttgactttctgatggccatagcaattaagcagccatcagtgtagtaccactaatgtaattgagacagggagtagactttcattggggcagttggactgcagtcttttgttgctcaggggtaagttagaggcaatcaaactgtttcaggtggtgagtgaaacttaagggatggtagaaaattagagacattcccattggatatgtagaaagtactctgatctgtagtgaagaacttaagtgaagatgcctaggactctgcccagttgagttcagaggaagctctcccagctttgaaattagacttgctttgcgaggaagacttcacctctaaagatgcaccaattgttttctctgagcaggttccaaaaagtagcattttttttttaatagacacatatagtaatgagctgaaaatactgagcttaatgtctcttgcctggtctttgtggtgaattctaatgtgtgattagcaagcatatgttctgattattgataaattgctgtatgtcaatcagtggaatactctactgcagttctgagaattgtctccaatattaaggcttaaataaacaagaggtagtgagataaattgaaaacctcttttgggatcgcttcctccaatagtgtaattattcctgtagttcctcctttcattcaaacctctgcaggaagtacagaatttagtacatactaattgaaggagcttttggctttctgatgctactaatattaacagtagtactcacttgagtaatttaaatgagagaatattgaatgtggcatttaattcctttcatttggcccagtgtgctgtcagtcagcagcaaatgtactttcatgctgaattatatattaatgtcctgttaatatcagttaatgttctttttactgttttagttttttttaaaaaaaaaactaacagctgtcaaaaaatgaaaatgtagtatttgaataatattttttttcttttcaggtatggatttgtttcattcctggacaatgtggatgttcaaaagatagtagaagtaagctctttatgtcttaagttgtcagaagaaccttctgtatgaaggttgtaggtgtggttaggggataccagtcccaactgagaaaataaaaaagactagaagtgccccaaagtaaacttgcttaaatattgttgtgatttaacccagcagattgtgaagtaccatgtagtattttcctcactgcactccatcttgctgaaaaactcgagccatccggaagatctggcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggc

SEQ ID NO: 38 - DNA, 22 bp, Artificial Sequence
  Description of Artificial Sequence Synthetic oligonucleotide
cccaaatataacacgcttcact

SEQ ID NO: 39 - DNA, 23 bp, Artificial Sequence
  Description of Artificial Sequence Synthetic oligonucleotide
gaaatgaattattttctggcgac

SEQ ID NO: 40 - DNA, 20 bp, Artificial Sequence
  Description of Artificial Sequence Synthetic oligonucleotide
agctctttctcgattccgtg

SEQ ID NO: 41 - DNA, 20 bp, Artificial Sequence
  Description of Artificial Sequence Synthetic oligonucleotide
gggtagacacaagctgagcc

SEQ ID NO: 42 - DNA, 20 bp, Artificial Sequence
  Description of Artificial Sequence Synthetic oligonucleotide
caactatcaggctccaccac

SEQ ID NO: 43 - DNA, 20 bp, Artificial Sequence
  Description of Artificial Sequence Synthetic oligonucleotide
ctcagacggttttcagggtt

SEQ ID NO: 44 - DNA, 20 bp, Artificial Sequence
  Description of Artificial Sequence Synthetic oligonucleotide
aggctatgggatgatgcaag

SEQ ID NO: 45 - DNA, 20 bp, Artificial Sequence
  Description of Artificial Sequence Synthetic oligonucleotide
gtaggtaggcgatccgttca

SEQ ID NO: 46 - DNA, 20 bp, Artificial Sequence
  Description of Artificial Sequence Synthetic oligonucleotide
cgagaccaacgtgaagggaa

SEQ ID NO: 47 - DNA, 20 bp, Artificial Sequence
  Description of Artificial Sequence Synthetic oligonucleotide
cagacccggacaacgtcttt

SEQ ID NO: 48 - DNA, 20 bp, Artificial Sequence
  Description of Artificial Sequence Synthetic oligonucleotide
ctctggggctcacctacaag

SEQ ID NO: 49 - DNA, 20 bp, Artificial Sequence
  Description of Artificial Sequence Synthetic oligonucleotide
agccctggtgaaatgtaggg

SEQ ID NO: 50 - DNA, 20 bp, Artificial Sequence
  Description of Artificial Sequence Synthetic oligonucleotide
agctctcatctcaaggcaca

SEQ ID NO: 51 - DNA, 20 bp, Artificial Sequence
  Description of Artificial Sequence Synthetic oligonucleotide
ggaaagatccactgcttcca

SEQ ID NO: 52 - DNA, 22 bp, Artificial Sequence
  Description of Artificial Sequence Synthetic oligonucleotide
agcacaggtggtgaacgaacca

SEQ ID NO: 53 - DNA, 23 bp, Artificial Sequence
  Description of Artificial Sequence Synthetic oligonucleotide
tccaggcctcttgatgctaccga

SEQ ID NO: 54 - DNA, 925 bp, Artificial Sequence
  Description of Artificial Sequence Synthetic polynucleotide
ctcctacctgcctcttcttctgacagagacgtccccttgatcatacagtagatgtctgagttttcatatggctgcagctctgcagggacagaaagagcacgagggtgtgtggaaggagtggggcagactcgaacagcagctcaggcactgacctgctctccccccaccccagccccgcagccgtacctgtgctggagccctacctatgtaatggtcctcctcggctttgatgggcacggccttccttctcctgggaagacaaagggacggtctgaatttgggaacaggctgacatggccctgctgaacccggtggcagtggaagaagagctctgcacactctgtgcccaggcctctaccccattggcaggcattgcccgtcagcaccccaagttgctgacctgcagcagtggccacaggtctcccagcacacacagcccctgccccaaccctgaggatgacgctcagatgaccccattagcagccctacatctgagccacggttgccatctgagcatcagccggccagcagcacccctgttgcttgaaaagggtgggagcagagcaggaagcctgctctgtcaccagccaccccagaagcacagcgtggcctgaggcagcacagcagctccaagtcccagggctactcacctggacagcacgaaccacacgatcaccgcagagagaatgaccatcactgagagcacaaccaccactgtgactgctgtccatggccaggagcccagaaactgccctgaaactaacacacagccagcagtgggtgtcacagcctggggtgtgtgcccaggccttggcatcaagtgtgtgcacgcgtgcaggtgtgtgtgtccaggatccacagttctgtggggacaggcagcaagtcacagccatgtgctgccttcagctctaatgggcagagaaga

SEQ ID NO: 55 - DNA, 20 bp, Artificial Sequence
  Description of Artificial Sequence Synthetic oligonucleotide
ctcctacctgcctcttcttc

SEQ ID NO: 56 - DNA, 20 bp, Artificial Sequence
  Description of Artificial Sequence Synthetic oligonucleotide
tcttctctgcccattagagc

SEQ ID NO: 57 - DNA, 24 bp, Artificial Sequence
  Description of Artificial Sequence Synthetic oligonucleotide
aagacaaagggacggtctgaattt

SEQ ID NO: 58 - DNA, 25 bp, Artificial Sequence
  Description of Artificial Sequence Synthetic oligonucleotide
tgaaactaacacacagccagcagtg

SEQ ID NO: 59 - DNA, 20 bp, Artificial Sequence
  Description of Artificial Sequence Synthetic oligonucleotide
gctggcattcgctatggagg

SEQ ID NO: 60 - DNA, 20 bp, Artificial Sequence
  Description of Artificial Sequence Synthetic oligonucleotide
ttctgaggagcaggcgtgga

SEQ ID NO: 61 - DNA, 20 bp, Artificial Sequence
  Description of Artificial Sequence Synthetic oligonucleotide
aatggagccatagcagagcc

SEQ ID NO: 62 - DNA, 19 bp, Artificial Sequence
  Description of Artificial Sequence Synthetic oligonucleotide
tgcaggaagcactgcgaag

SEQ ID NO: 63 - DNA, 28 bp, Artificial Sequence
  Description of Artificial Sequence Synthetic oligonucleotide
ctatggaggaggactgggacacggagct

SEQ ID NO: 64 - protein, 8 aa, Artificial Sequence
  Description of Artificial Sequence Synthetic peptide
MEEDWDTE

SEQ ID NO: 65 - DNA, 28 bp, Artificial Sequence
  Description of Artificial Sequence Synthetic oligonucleotide
ctatggatggtgagctgtgtccagggga

(19) United States
(12) Patent Application Publication
(10) Pub. No.: US 2024/0052304 A1
(43) Pub. Date: Feb. 15, 2024

(54) STERILE AVIAN EMBRYOS, PRODUCTION AND USES THEREOF

(71) Applicant: THE STATE OF ISRAEL MINISTRY OF AGRICULTURE & RURAL DEVELOPMENT, AGRICULTURAL RESEARCH ORGANIZATION, Rishon Lezion (IL)

(72) Inventors: Yuval CINNAMON, Jerusalem (IL); Enbal BEN-TAL COHEN, Rehovot (IL)

(73) Assignee: THE STATE OF ISRAEL MINISTRY OF AGRICULTURE & RURAL DEVELOPMENT, AGRICULTURAL RESEARCH, Rishon Lezion (IL)

(21) Appl. No.: 18/269,131

(22) PCT Filed: Dec. 30, 2021
(86) PCT No.: PCT/IL2021/051552

§ 371 Date: Jun. 22, 2023

Related U.S. Application Data
(60) Provisional application No. 63/132,753, filed on Dec. 31, 2020.

(51) Int. Cl. C12N 5/0735; A01K 67/027

(52) U.S. Cl. CPC C12N 5/0611; A01K 67/0275; C12N 2510/00; A01K 2217/075; A01K 2227/30

(57) ABSTRACT

The present disclosure relates to deoxyribonucleic acid (DNA) editing agents, and their use in preparing DNA-edited cells and birds. The present disclosure further relates to gene-edited or genetically modified avians and gene-edited or genetically modified avian primordial germ cells (PGCs) for producing gene-edited or genetically modified avians (birds) that can serve as surrogate hosts for donor PGCs. The present disclosure further relates to methods for producing avian strains that can produce viable embryos and offspring, in both sexes, and for their subsequent use as surrogate hosts for donor PGCs.

### SEQUENCE LISTING STATEMENT

The instant application contains a Sequence Listing which has been submitted electronically in ASCII format and is hereby incorporated by reference in its entirety. Said ASCII copy, created on Dec. 28, 2021, is named P-592523-PC SL.txt and is 70,092 bytes in size.

### FIELD OF INTEREST

The present disclosure relates to deoxyribonucleic acid (DNA) editing agents, and their use in preparing DNA-edited cells and birds. The present disclosure further relates to sterile gene-edited or genetically modified avians and gene-edited or genetically modified avian primordial germ cells (PGCs) for producing sterile gene-edited or genetically modified avians (birds) that can serve as surrogate hosts for donor PGCs. The present disclosure further relates to methods for producing avian strains that can produce viable sterile embryos and offspring, in both sexes, and for their subsequent use as surrogate hosts for donor PGCs.

### BACKGROUND OF THE INVENTION

The poultry industry accounts for over one-third of dietary protein production worldwide. With respect to chickens, one of the most popular poultry species, this FIG. originates mainly from two sources, table eggs from layer-type hens and broiler meat. Increase in world population and predicted demand for food by 2050, requires adaptation to food production to sustain the next generations, in an economical and affordable manner. It is estimated that >65 billion broilers are slaughtered annually worldwide. Notably, this is >10 times more than all the rest of the livestock animals altogether and demand for broiler meat is constantly growing worldwide. Significant increase in broiler egg production per hen would lower the numbers of required hens, thereby improving productivity, animal welfare issues and contribute for worldwide sustainability. Moreover, the production cost and footprint per egg will decrease.

Within the last 60 years of genetic selection, the broiler and layer have become extremely distinguished breeds, exploiting the best performances according to market's needs. While the modern broiler breeder hen produces ˜120-140 eggs per year, the modern layer-type breeder hen can lay more than 340 eggs, for significantly less feed per egg. Moreover, while commercial layer-type breeder hens will reach sexual maturity and start laying eggs at the age of 16-18 weeks, and can lay eggs until >72 weeks of age, the commercial broiler breeder will start laying eggs at the age of 26-28 weeks until reaching 60-62 weeks of age. These figures reflect demonstrate the tremendous advantage of the layer-type hen in terms of efficiency and reproduction.

One problem within the poultry industry is that male chicks, as non-layers, are an inevitable byproduct of the industry, and thus, they are manually sorted and culled using labor-intensive techniques. Sex determination in chickens is based on combinatorial segregation of the sex chromosomes Z and W in the hen's gametes. Each male chick harbors a Z chromosome which segregates from its mother hen.

Chickens and other avians (birds) reproduce by eggs, which, in most species, are fertilized internally in the female and then coated with a shell prior to laying. The avian embryo then incubates in the egg externally until hatching. Typically, one or both parents will participate in the incubation process.

The gametes in adults (sperm in males and eggs in females) originate from a unique population of embryonic cells called Primordial Germ Cells (PGCs). In chickens, the first PGCs are identified in the center of the embryonic blastoderm at oviposition. Within the first 24 h of incubation, the PGCs migrate to an extra embryonic region—the Germinal Crescent, at the anterior side of the embryo. As the blood system develops, the PGCs migrate through the blood circulation and colonize the Germinal Ridge, the anlage of the embryonic gonads. In chickens, two symmetrical embryonic gonads are formed for both sexes and by the ninth day of incubation, in females, the right gonad regresses, while the left gonad develops into a single ovary. In males, both embryonic gonads develop into the testes. Reaching sexual maturity, the PGCs give rise to gametes—ovulating eggs and sperm in females and males, respectively. Since PGCs are not somatic cells, they have no role other than hereditary. In the absence of PGCs, no eggs or sperm are formed, which renders the organism sterile, but otherwise healthy.

Thus, ablating PGCs or affecting their ability to differentiate to gametes results in sterility. At early stages of embryogenesis, PGCs relocate to the embryonic gonads through the bloodstream, where they can be collected from, and returned to.

In addition, PGCs can readily undergo various types of genetic transformation, including, but not limited to, gene silencing, gene misexpression, gene overexpression, and transgenesis, whereas foreign DNA elements, which otherwise do not exist in the genome, can be inserted in a random or targeted manner into the genome. These modifications can improve, amongst other traits, agricultural performance, health, disease resistance, resilience to various stress conditions, and behavioral characteristics, and they can also be used to introduce traits which do not naturally exist in chickens.

Moreover, cryopreservation of chicken gametes has presented a long-standing challenge, because the huge ovulated egg cannot successfully be restored from cryopreservation, inseminated, and restored back to the infundibulum of the oviduct, and sperm cryopreservation is highly unreliable. For many decades, poultry breeding companies created thousands of invaluable genetic colonies having diverse genetic backgrounds. Because gamete cryopreservation is not reliably and efficiently feasible in chickens, these flocks must be kept alive. Even when they are not regularly in use, the vast majority of the colonies are kept merely for genetic records, backup in case of a catastrophe, and genetic diversity preservation. This situation imposes vast economic losses and harms livestock welfare. Additionally, numerous endangered chicken breeds, non-commercial and wild breeds cannot be cryopreserved, thus increasing the chance of losing potential genetic diversity and increasing breed extinctions.

Unlike gamete cryopreservation, cryopreservation of PGCs is readily feasible. PGCs can be collected at various embryonic stages starting from the freshly laid egg, the germinal crescent, the bloodstream, or directly from the gonads. When sufficient quantities of PGCs are obtained, either by direct collection (e.g., from the gonads) or following culturing, PGCs can be cryopreserved in liquid nitrogen for many years.

Generating genome edited chicken breeds is a multi-step process. Following genomic transformation, the genome-modified PGCs are currently injected to a surrogate recipient host embryo alongside to its endogenous PGCs, thereby giving rise to “chimera” and the two populations of PGCs colonize the gonad. The ratio between the endogenous and modified PGCs in the gonads, and their potential to give rise to functional gametes, is reflected in the germline transmission, which is variable. Namely, in the case of males, in this example, this will be the ratio between modified and wild-type sperm cells, in a given semen sample, that can fertilize eggs. Low germline transmission ratio results in months of laborious screening for founder chicks which originate from modified PGCs. Alternatively, modification can be achieved by injecting viruses into the blastoderm, but this method is highly inefficient and inaccurate.

It would be desirable to have compositions and methods for producing modified PGCs and for obtaining sterile birds therefrom. It would also be desirable to have compositions and methods for transforming sterile surrogate host birds in order to have them, upon sexual maturity, produce gametes originating from a selected genetic background of interest.

### SUMMARY OF THE INVENTION

The compositions and methods provided herein are directed to the ability to collect PGCs, culture them and back transplant donor PGCs to host chimera embryos that will hatch and grow to sexual maturity. Upon sexual maturity the host avians will produce gametes originating from the genetic background of the transplanted PGCs.

In some aspects, disclosed herein is a gene-edited or genetically modified avian primordial germ cell (PGC) comprising: a first genetic modification on a chromosome, the first genetic modification modifying a trait in the PGC or in the gene-edited or genetically modified avian produced by the PGC or a combination thereof, when compared to an isogenic PGC or isogenic avian lacking the first genetic modification, the modified trait inducing sterility in the gene-edited or genetically modified avian produced by the PGC without impairing viability of the gene-edited or genetically modified avian produced by the PGC.

In related aspects, disclosed herein is a sterile gene-edited or genetically modified avian embryo comprising gene-edited or genetically modified avian cells, each gene-edited or genetically modified avian cell comprising: a first genetic modification on the same chromosome, the first genetic modification modifying a trait in the gene-edited or genetically modified avian embryo or in the PGC produced by the gene-edited or genetically modified avian embryo as an adult or a combination thereof, when compared to an isogenic avian embryo or a PGC produced by an isogenic avian embryo as an adult lacking the first genetic modification, the modified trait inducing sterility in a gene-edited or genetically modified avian embryo, comprising the gene-edited or genetically modified avian cell, as an adult without impairing viability, or in a gene-edited or genetically modified avian offspring produced by the PGC produced by the gene-edited or genetically modified avian embryo as an adult without impairing viability of the gene-edited or genetically modified avian offspring produced by the PGC.

In a related aspect, disclosed herein is a sterile gene-edited or genetically modified avian comprising gene-edited or genetically modified avian cells, each gene-edited or genetically modified avian cell comprising: a first genetic modification on the same chromosome, the first genetic modification modifying a trait in the gene-edited or genetically modified avian or in the PGC produced by the gene-edited or genetically modified avian or a combination thereof, when compared to an isogenic avian or a PGC produced by an isogenic avian lacking the first genetic modification, the modified trait inducing sterility in a gene-edited or genetically modified avian comprising the gene-edited or genetically modified avian cell without impairing viability, or in a gene-edited or genetically modified avian offspring produced by the PGC produced by the gene-edited or genetically modified avian without impairing viability of the gene-edited or genetically modified avian offspring produced by the PGC.

In another aspect, disclosed herein is a deoxyribonucleic acid (DNA) editing system comprising: a first agent comprising a first nucleic acid sequence for eliciting a sterile phenotype in a gene-edited or genetically modified avian operatively linked to a recombinase recognition site and a sequence for directing the first nucleic acid sequence to a targeted gene of interest (GOI) on a chromosome of interest of a primordial germ cell (PGC); a second agent comprising a second nucleic acid sequence, the second nucleic acid sequence encoding a recombinase enzyme and a sequence for directing the second nucleic acid sequence to the targeted GOI on the chromosome of interest of the PGC.

In yet another aspect, disclosed herein is a method for producing a sterile gene-edited or genetically modified avian, the method comprising: obtaining a primordial germ cell (PGC) from an avian; stably integrating into a targeted gene of interest (GOI) on a chromosome of interest in the PGC a first exogenous polynucleotide which is operatively linked to a recombinase recognition site, the first exogenous polynucleotide eliciting a sterility-inducing phenotype in the PGC or in a gene-edited or genetically modified avian derived from the PGC, and stably integrating into the targeted GOI on the chromosome of interest in the PGC a second exogenous polynucleotide encoding a recombinase enzyme: wherein the first exogenous polynucleotide comprises a mutated or null GOI sequence or fragment thereof or encodes an endonuclease enzyme that can carry out genome editing; or wherein insertion of the first exogenous polynucleotide in the chromosome of interest modifies or disrupts the targeted GOI, the targeted GOI having: an isolated function specific to a PGC; or a function specific to gametogenesis, gamete maturation, or gamete function in the gene-edited or genetically modified avian; producing pure PGC colonies, the PGC colonies comprising the first exogenous polynucleotide and the second exogenous polynucleotide; transplanting a pure PGC colony to a male chick embryo to produce a chimera male chick embryo and transplanting a pure PGC colony to a female chick embryo to produce a chimera female chick embryo; hatching and rearing the chimera founder chick to sexual maturity as a chimera founder adult avian; screening the chimera founder adult avian to verify heterozygosity for the edited GOI; breeding a male chimera founder adult avian having heterozygosity for the edited GOI with a female chimera founder adult avian having heterozygosity for the edited GOI to produce progeny embryos; and identifying a sterile homozygotic embryo from the progeny embryos.

### BRIEF DESCRIPTION OF THE DRAWINGS

The patent or application file contains at least one drawing executed in color. Copies of this patent or patent application publication with color drawing(s) will be provided by the Office upon request and payment of the necessary fees.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1B are schematics depicting an example of genetic mutation to produce a mutation for producing a sterile avian embryo. FIG. 1A is a schematic depicting a chromosomal map of Deleted in AZoospermia-Like (DAZL) (DAZL Locus on Chromosome 2: 34429592 . . . 34442888; GRCg6a). The transcript is depicted as the solid blue arrow, while the corresponding complementary deoxyribonucleic acid (cDNA) sequence is shown with the relative positions of the exons shown in gray and the relative positions of the introns indicated by dashed lines. The relative positions of the 5′ homology arm (5′ HA) and the 3′ homology arm (3′ HA) are indicated in red. The overlapping region and flanking regions of the 5′HA and Exon2 are shown (left) with the DNA sequence (black; SEQ ID NO: 25) and corresponding protein sequence (blue; SEQ ID NO: 26) of Exon2 as shown. Similarly, the overlapping region and flanking regions of Exon3 and the 3′HA are shown (right) with the DNA sequence (black; SEQ ID NO: 27) and corresponding protein sequence (blue; SEQ ID NO: 28) of Exon3 as shown. The positions of the CRISPR1 (green; SEQ ID NO: 1), CRISPR2 (light blue; SEQ ID NO: 2), and CRISPR3 (pink; SEQ ID NO: 3) single guide ribonucleic acid (sgRNA) sequences are also indicated. FIG. 1B is a schematic demonstrating how the coding sequence of the reporter gene mCherry is subsequently introduced into the site using the targeting vector (TV) and replaces the coding sequence of DAZL. The polyadenylation site following the coding sequence of mCherry is shown (gold, PA). The DNA sequences (SEQ ID NOs: 29 and 31) are shown in black. The wild-type DAZL protein sequence is shown in blue for the 5′ HA, while the replacement mCherry marker protein sequence is shown in red (SEQ ID NO: 30). The wild-type DAZL protein sequence is also shown in blue for the 3′HA (SEQ ID NO: 32). The overall sequence of the pX3361-DAZL-CRISPR1 plasmid is SEQ ID NO: 10.

FIG. 2 is a schematic depicting the pX3361-DAZL-CRISPR1 plasmid (9292 bp) map. The locations of CRISPR sequences, the chimeric guide RNA scaffold, and the U6 terminator are indicated, and the CRISPR 1-3 DNA sequences are shown (inset; SEQ ID NO: 1-3). The overall sequence of the pX3361-DAZL-CRISPR1 with SEQ ID NO: 1 plasmid is SEQ ID NO: 10. The overall sequence of the pX3361-DAZL-CRISPR2 with SEQ ID NO: 2 plasmid is SEQ ID NO: 33. The overall sequence of the pX3361-DAXL-CRISPR3 with SEQ ID NO: 3 plasmid is SEQ ID NO: 34.

FIG. 3 is a schematic depicting the pJet1.2 DAZL-mCherry targeting vector (TV) plasmid (6967 bp) map. The locations of the 5′ HA and mCherry sequences are indicated, and a portion of the coding region of the 5′ HA-mCherry sequence is shown (inset). The DNA sequence is shown in black (SEQ ID NO: 35), while the corresponding encoded protein is shown in red (SEQ ID NO: 36). The overall sequence of the pJet1.2 DAZL-mCherry TV plasmid is SEQ ID NO: 37.

FIGS. 4A-4C depict polymerase chain reaction (PCR) validation of the correct (homologous recombination) HR. FIG. 4A depicts a diagram of a PCR validation method to confirm the integrity of the wild-type (WT) allele, with the forward (primer 5, P5) and reverse (primer 6, P6) primer locations as shown, flanking the CRISPR sites. This PCR product was subsequently confirmed by sequencing. FIG. 4B depicts a diagram of a PCR validation method to confirm correct HR and integration of the target vector, using two sets of primers (for the 5′ and the 3′ integration sites), the locations of which are shown in relation to the knock-in allele. For the 5′ integration site, the forward primer (primer 1, P1) is located upstream and outside the 5′ HA and the reverse primer (primer 2, P2) is located in the mCherry gene, as shown. For the 3′ integration site, the forward primer (primer 3, P3) is located in the mCherry region and the reverse primer (primer 4, P4) is located downstream and outside the 3′ HA region. FIG. 4C shows the result of gel electrophoresis of PCR products of the reactions depicted in FIG. 4B. The predicted PCR product sizes of the reactions are 1803 bp (3′ integration site; center lane) and 1794 bp (5′ integration site; right lane), with gel electrophoresis bands confirming these sizes of the actual PCR products, as shown in comparison with the DNA ladder marker (left lane).

FIGS. 5A-5E are photographs demonstrating the process of colonization of modified PGCs within surrogate gonads of a chick embryo. In FIG. 5A, viable dividing mCherry positive cells (red) were observed in culture. mCherry positive cells were collected using fluorescence-activated cell sorting (FACS) and subsequently grown to form stable colonies (FIG. 5B). Genomic DNA from these colonies was analyzed to further confirm the homologous recombination (HR) (using the method shown in FIGS. 4B-4C). Recombinant PGCs were injected into the bloodstream of chick embryos (58-70 hours [h] after laying). FIG. 5C shows a representative chick embryo of a subset of chick embryos analyzed under fluorescent microscope 48 h post-injection. mCherry positive PGCs are shown to be located in the anlage of the embryonic gonads, in the genital ridge (arrows). FIG. 5D shows a representative chick embryo of a subset of chick embryos dissected, ventral-side up, to observe the gonads (delineated with a red line) 8 days (d) post-injection. FIG. 5E shows higher magnification of the inset region denoted by the blue rectangle in FIG. 5D. FIG. 5E, Left panel: fluorescence on the green channel as negative control; middle panel: fluorescence on the red channel showing mCherry-positive cells; and right panel: showing overlapping merged image of both channels, taken under a fluorescent microscope. The embryonic gonads, delineated with a white line, show female phenotype gonads in which numerous mCherry positive cells have been colonized (red dots).

FIG. 6 is a flow chart depicting an embodiment of a method for producing a sterile avian embryo.

FIG. 7 presents a photograph of a surrogate layer-breed hen and the broiler-breed male chick hatched from an egg she laid. The surrogate hen, shown on the right, reached a weight of 2.6 kg by the age of 32 weeks. The hatched male chick was reared and reached a weight of 3.3 kg, by the age of 7 weeks, as shown on the left.

FIGS. 8A-8B presents a genetic motherhood test of the PGCs injected, male broiler chick hatched, surrogate layer hen, and a male half layer sibling. FIG. 8A shows a partial sequence chromatogram of SEQ ID NO: 57: AAGACAAAGGGACGGTCTGAATTT that includes the dbSNP:rs736292769 C/T marked in blue. FIG. 8B shows a partial sequence chromatogram of SEQ ID NO: 58: TGAAACTAACACACAGCCAGCAGTG that includes dbSNP:rs731066568 C/A marked in blue. Both SNPs in the PGCs line and the broiler chicks are identical and differ from the SNPs of the surrogate hen and sibling half layer chick, confirming that the broiler chick originated from an egg derived from the injected PGCs.

FIG. 9 presents the results of PCR analysis to identify the genotype of pure colonies. Genomic DNA from 11 colonies (C1-C11) was extracted, and used as template for PCR with primers SEQ ID NO: 61 (forward) and SEQ ID NO: 62 (reverse). The 1 kb DNA ladder is shown on the left with the indicated bp size, followed by no-template negative control (NTC) lane. The white and black arrows indicate the two alleles present in the colony.

FIG. 10 presents the DNA sequences of the two alleles of colony C11 (of FIG. 9), which were sequenced separately. The chromatogram confirms that one allele is WT (SEQ ID NO: 63) and the second allele harbors a deleterious 70 bp deletion that renders the DDX4 copy on this allele null (SEQ ID NO: 65). The WT amino acid translation from the first methionine is provided in SEQ ID NO: 64.

It will be appreciated that for simplicity and clarity of illustration, elements shown in the FIGS. have not necessarily been drawn to scale. For example, the dimensions of some of the elements may be exaggerated relative to other elements for clarity. Further, where considered appropriate, reference numerals may be repeated among the FIGS. to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Before explaining at least one embodiment of the disclosure in detail, it is to be understood that the disclosure is not necessarily limited in its application to the details set forth in the following description or exemplified by the Examples. The disclosure encompasses other embodiments or can be practiced or carried out in various ways.

Sterile avians are beneficial for several applications and their usefulness is relevant to both poultry (e.g., layer and broiler chickens) and game industries, as well as for research and for wild avian breed and species conservation. Provided herein are sterile gene-edited or genetically modified avians and gene-edited or genetically modified avian primordial germ cells (PGCs) for producing sterile gene-edited or genetically modified avians (birds) that can serve as surrogate hosts for donor PGCs. Also provided herein are deoxyribonucleic acid (DNA) editing systems and methods for producing avian strains that can produce viable sterile embryos and offspring, in both sexes, and for their subsequent use as surrogate hosts for donor PGCs.

In some aspects, disclosed herein is a gene-edited or genetically modified avian primordial germ cell (PGC) comprising: a first genetic modification on a chromosome, the first genetic modification modifying a trait in the PGC or in the gene-edited or genetically modified avian produced by the PGC or a combination thereof, when compared to an isogenic PGC or isogenic avian lacking the first genetic modification, the modified trait inducing sterility in the gene-edited or genetically modified avian produced by the PGC without impairing viability of the gene-edited or genetically modified avian produced by the PGC.

In some embodiments, the first genetic modification comprising modification of a gene having an isolated function specific to a PGC or modification of a gene having a function specific to gametogenesis, gamete maturation, or gamete function. In some embodiments, a first genetic modification eliminates a function specific to PGCs. In some embodiments, a first genetic modification reduces a function specific to PGCs. In some embodiments, a first genetic modification eliminates a function specific to gametogenesis, gamete maturation, or gamete function. In some embodiments, a first genetic modification reduces a function specific to gametogenesis, gamete maturation, or gamete function.

In some embodiments, the modification of the gene having an isolated function specific to a PGC reduces or inhibits survival, maturation, or differentiation of a PGC derived from the gene-edited or genetically modified avian. In some embodiments, the modification of a gene having a function specific to gametogenesis, gamete maturation, or gamete function reduces or inhibits gametogenesis, meiosis, gamete function, or gamete fertilization in the gene-edited or genetically modified avian.

In some embodiments, the first genetic modification comprises modification of a gene encoding a protein, said protein comprising a Zona Pellucida Binding Protein 1/2 (ZPBP1/2) protein, Cyclin-Dependent Kinases Regulatory Subunit 2 (CKS2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis Specific With Coiled-Coil Domain (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein. In some embodiments, the first genetic modification comprises modification of a gene encoding a Deleted In Azoospermia-Like (DAZL) protein. In some embodiments, the first genetic modification comprises modification of a gene encoding a Deleted In Azoospermia-Like (DAZL) protein. In some embodiments, the first genetic modification comprises modification of a gene encoding a DEAD-Box Helicase 4 (DDX4) protein. A skilled artisan would appreciate that there may be redundancy mechanisms or

shared activities between proteins. Therefore, in some embodiments, the first genetic modification comprises modification of a combination of genes. In some embodiments, the first genetic modification comprises modification of a combination of at least 2 genes, said genes comprising those encoding a protein comprising a Zona Pellucida Binding Protein 1/2 (ZPBP1/2) protein, Cyclin-Dependent Kinases Regulatory Subunit 2 (CKS2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis Specific With Coiled-Coil Domain (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein. In some embodiments, a combination comprises mutations in two genes. In some embodiments, a combination comprises mutations in more than two genes. In some embodiments, a combination comprises mutations in at least 2, 3, 4, or 5 genes. In some embodiments, a combination comprises a combination of mutations such that the function specific to gametogenesis, gamete maturation, or gamete function is eliminated. In some embodiments, a combination comprises a combination of mutations such that the function specific to gametogenesis, gamete maturation, or gamete function is reduced.

The gene-edited or genetically modified avian PGC disclosed herein, wherein the chromosome is an autosomal chromosome.

In some embodiments, the modified trait induces sterility in a male gene-edited or genetically modified avian produced by the PGC and a female gene-edited or genetically modified avian produced by the PGC.

In some embodiments, a second genetic modification is on the same chromosome as the first genetic modification, the second genetic modification encoding a marker detectable in the PGC, in the gene-edited or genetically modified avian produced by the PGC, or in a PGC produced by the gene-edited or genetically modified avian produced by the PGC, wherein the marker is detectable in the cytoplasm of the PGC. In some embodiments, the marker is a fluorescent protein, a luminescent protein, or a chromoprotein. In some embodiments, the marker is (a) a fluorescent protein comprising Green Fluorescent Protein (GFP), Enhanced Green Fluorescent Protein (EGF), Emerald, Superfolder GFP, Azami Green, mWasabi, Tag-Green Fluorescent Protein (TagGFP), Turbo-Green Fluorescent Protein (TurboGFP), mNeonGreen, mUKG, acGFP, ZsGreen, Cloverm Sapphire, T-Sapphire, Enhanced Blue Fluorescent Protein (EBFP), Enhanced Blue Fluorescent Protein 2 (EBFP2), Azurite, Tag-Enhanced Blue Fluorescent Protein (TagBFP), mTagBFP, mKalamal, Cyan Fluorescent Protein (CFP), mCFP, Enhanced Cyan Fluorescent Protein (ECFP), mECFP, Cerulean, SCFP3A, mTurquoise, mTurquoise2, CyPet, AmCyan1, Midori-Ishi Cyan, Tag-Cyan Fluorescent Protein (TagCFP), mTFP1 (Teal), Yellow Fluorescent Protein (YFP), Enhanced Yellow Fluorescent Protein (EYFP), Super Yellow Fluorescent Protein (SYFP), Topaz, Venus, Citrine, mCitrine, YPet, Tag-Yellow Fluorescent Protein (TagYFP), Turbo-Yellow Fluorescent Protein (TurboYFP), Phi-Yellow Fluorescent Protein (PhiYFP), ZsYellow1, mBanana, Kusabira Orange, Kusabira Orange2, mOrange, mOrange2, dTomato, dTomato-Tandem, Red Fluorescent Protein (RFP), Turbo-Red Fluorescent Protein (TurboRFP), TurboFP602, TurboFP635, Tag-Red Fluorescent Protein (RFP), TagRFP-T, DsRed, DsRed2, DsRed-Express (T1), DsRed-Monomer, mTangerine, mKeima-Red, mRuby, mRuby2, mApple, mStrawberry, AsRed2, mRFP1, J-Red, mCherry, mKate (TagFP635), mKate2, HcRed1, mRaspberry, dKeima-Tandem, HcRed-Tandem, mPlum, mNeptune, NirFP, Sinus, TagFRP657, AQ143, Kaede, KikGR1, PX-CFP2, mEos2, IrisFP, mEOS3.2, PSmOrange, PAGFP, Dronpa, Allowphycocyanin, GFPuv, R-Phycoerythrin (RPE), Peridinin Chlorophyll (PerCP), P3, Katusha, B-Phycoerythrin (BPE), and mKO; or (b) a chromoprotein comprising ShadowR, Stichodactyla gigantea (sgBP), Heteractis crispa (hcCP), Anemonia sulcata (asCP), Cnidopus japonicus (cjBlue), or Goniopora tenuidens (gtCP). In some embodiments, the marker is mCherry.

In some embodiments, the gene-edited or genetically modified avian PGC is derived from an avian of the Galliformes order, the Anseriformes order, the Otidiformes order, the Columbiformes order, or the Struthioniformes order. In some embodiments, the gene-edited or genetically modified avian PGC derived from an avian of the Galliformes order comprises the Phasianidae family or the Numididae family. In some embodiments, the gene-edited or genetically modified avian PGC is derived from an avian of the Phasianidae family comprising the Gallus genus or the Meleagris genus. In some embodiments, the gene-edited or genetically modified avian PGC is derived from an avian of the Anseriformes order comprising the Anatidae family, the Anseranatidae family, or the Anhimidae family. In some embodiments, the gene-edited or genetically modified avian PGC is derived from an avian of the Otidiformes order comprising the Otididae family. In some embodiments, the gene-edited or genetically modified avian PGC is derived from an avian of the Columbiformes order comprising the Columbidae family. In some embodiments, the gene-edited or genetically modified avian PGC derived from an avian of the Struthioniformes order comprising the Struthionidae family.

In related aspects, disclosed herein is a sterile gene-edited or genetically modified avian embryo comprising gene-edited or genetically modified avian cells, each gene-edited or genetically modified avian cell comprising: a first genetic modification on the same chromosome, the first genetic modification modifying a trait in the gene-edited or genetically modified avian embryo or in the PGC produced by the gene-edited or genetically modified avian embryo as an adult or a combination thereof, when compared to an isogenic avian embryo or a PGC produced by an isogenic avian embryo as an adult lacking the first genetic modification, the modified trait inducing sterility in a gene-edited or genetically modified avian embryo, comprising the gene-edited or genetically modified avian cell, as an adult without impairing viability, or in a gene-edited or genetically modified avian offspring produced by the PGC produced by the gene-edited or genetically modified avian embryo as an adult without impairing viability of the gene-edited or genetically modified avian offspring produced by the PGC.

In a related aspect, disclosed herein is a sterile gene-edited or genetically modified avian comprising gene-edited or genetically modified avian cells, each gene-edited or genetically modified avian cell comprising: a first genetic modification on the same chromosome, the first genetic modification modifying a trait in the gene-edited or genetically modified avian or in the PGC produced by the gene-edited or genetically modified avian or a combination thereof, when compared to an isogenic avian or a PGC produced by an isogenic avian lacking the first genetic modification, the modified trait inducing sterility in a gene-edited or genetically modified avian comprising the gene-edited or genetically modified avian cell without impairing viability, or in a gene-edited or genetically modified avian offspring produced by the PGC produced by the gene-edited or genetically modified avian without impairing viability of the gene-edited or genetically modified avian offspring produced by the PGC.

In another aspect, disclosed herein is a deoxyribonucleic acid (DNA) editing system comprising: a first agent comprising a first nucleic acid sequence for eliciting a sterile phenotype in a gene-edited or genetically modified avian operatively linked to a recombinase recognition site and a sequence for directing the first nucleic acid sequence to a targeted gene of interest (GOI) on a chromosome of interest of a primordial germ cell (PGC); a second agent comprising a second nucleic acid sequence, the second nucleic acid sequence encoding a recombinase enzyme and a sequence for directing the second nucleic acid sequence to the targeted GOI on the chromosome of interest of the PGC.

In some embodiments, the first nucleic acid sequence comprises a mutated or null GOI sequence or a fragment thereof or encodes an endonuclease enzyme that can carry out genome editing; or wherein insertion of the first nucleic acid sequence in the chromosome of interest modifies or disrupts the targeted GOI, the targeted GOI has: an isolated function specific to a PGC; or a function specific to gametogenesis, gamete maturation, or gamete function in the gene-edited or genetically modified avian. In some embodiments, the modification or disruption of the gene has an isolated function specific to a PGC reduces or inhibits survival, maturation, or differentiation of a PGC derived from the gene-edited or genetically modified avian. In some embodiments, the modification or disruption of a gene has a function specific to gametogenesis, game maturation, or gamete function reduces or inhibits gametogenesis, meiosis, gamete function, or gamete fertilization in the gene-edited or genetically modified avian.

In some embodiments, the modification or disruption of a gene comprises modification or disruption of a gene encoding a protein selected from the group consisting of Zona Pellucida Binding Protein 1/2 (ZPBP1/2) protein, Cyclin-Dependent Kinases Regulatory Subunit 2 (CKS2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis-Specific With Coiled-Coil Domain-Containing Protein (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein. In some embodiments, the modification or disruption of a gene comprises modification or disruption of a gene encoding a Deleted In Azoospermia-Like (DAZL) protein.

In some embodiments, the modification or disruption of a gene comprises modification or disruption of at least 2 genes, said genes comprising those encoding a protein selected from the group consisting of Zona Pellucida Binding Protein 1/2 (ZPBP1/2) protein, Cyclin-Dependent Kinases Regulatory Subunit 2 (CKS2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis-Specific With Coiled-Coil Domain-Containing Protein (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein. In some embodiments, the modification or disruption of a gene comprises modification or disruption of at least 2, 3, 4, or 5 genes

In some embodiments, the chromosome of interest is an autosomal chromosome.

In some embodiments, the sterile phenotype induces sterility in a male gene-edited or genetically modified avian produced by the PGC and a female gene-edited or genetically modified avian produced by the PGC.

In some embodiments, the sequence for directing the first nucleic acid sequence or the second nucleic acid sequence to the chromosome of interest of the PGC comprises: a left homology arm (LHA) nucleotide sequence that is substantially homologous to the 5′ region flanking a target gene locus in the chromosome of interest of the PGC; and a right homology arm (RHA) nucleotide sequence that is substantially homologous to the 3′ region flanking the target gene locus in the chromosome of interest of the PGC.

In some embodiments, the first nucleic acid sequence or the second nucleic acid sequence comprises a detectable marker. In some embodiments, the marker is a fluorescent protein, a luminescent protein, or a chromoprotein. In some embodiments, the marker is (a) a fluorescent protein comprising Green Fluorescent Protein (GFP), Enhanced Green Fluorescent Protein (EGF), Emerald, Superfolder GFP, Azami Green, mWasabi, Tag-Green Fluorescent Protein (TagGFP), Turbo-Green Fluorescent Protein (TurboGFP), mNeonGreen, mUKG, acGFP, ZsGreen, Cloverm Sapphire, T-Sapphire, Enhanced Blue Fluorescent Protein (EBFP), Enhanced Blue Fluorescent Protein 2 (EBFP2), Azurite, Tag-Enhanced Blue Fluorescent Protein (TagBFP), mTagBFP, mKalamal, Cyan Fluorescent Protein (CFP), mCFP, Enhanced Cyan Fluorescent Protein (ECFP), mECFP, Cerulean, SCFP3A, mTurquoise, mTurquoise2, CyPet, AmCyan1, Midori-Ishi Cyan, Tag-Cyan Fluorescent Protein (TagCFP), mTFP1 (Teal), Yellow Fluorescent Protein (YFP), Enhanced Yellow Fluorescent Protein (EYFP), Super Yellow Fluorescent Protein (SYFP), Topaz, Venus, Citrine, mCitrine, YPet, Tag-Yellow Fluorescent Protein (TagYFP), Turbo-Yellow Fluorescent Protein (TurboYFP), Phi-Yellow Fluorescent Protein (PhiYFP), ZsYellow1, mBanana, Kusabira Orange, Kusabira Orange2, mOrange, mOrange2, dTomato, dTomato-Tandem, Red Fluorescent Protein (RFP), Turbo-Red Fluorescent Protein (TurboRFP), TurboFP602, TurboFP635, Tag-Red Fluorescent Protein (RFP), TagRFP-T, DsRed, DsRed2, DsRed-Express (T1), DsRed-Monomer, mTangerine, mKeima-Red, mRuby, mRuby2, mApple, mStrawberry, AsRed2, mRFP1, J-Red, mCherry, mKate (TagFP635), mKate2, HcRed1, mRaspberry, dKeima-Tandem, HcRed-Tandem, mPlum, mNeptune, NirFP, Sinus, TagFRP657, AQ143, Kaede, KikGR1, PX-CFP2, mEos2, IrisFP, mEOS3.2, PSmOrange, PAGFP, Dronpa, Allowphycocyanin, GFPuv, R-Phycoerythrin (RPE), Peridinin Chlorophyll (PerCP), P3, Katusha, B-Phycoerythrin (BPE), and mKO; or (b) a chromoprotein comprising ShadowR, Stichodactyla gigantea (sgBP), Heteractis crispa (hcCP), Anemonia sulcata (asCP), Cnidopus japonicus (cjBlue), or Goniopora tenuidens (gtCP). In some embodiments, the marker is mCherry.

In some embodiments, the first nucleic acid sequence comprises any one of the sequences set forth in SEQ ID NO: 10, SEQ ID NO: 33, or SEQ ID NO: 34; and the second nucleic acid sequence comprises sequences set forth in SEQ ID NO: 37.

In yet another aspect, disclosed herein is a method for producing a sterile gene-edited or genetically modified avian, the method comprising: obtaining a primordial germ cell (PGC) from an avian; stably integrating into a targeted gene of interest (GOI) on a chromosome of interest in the PGC a first exogenous polynucleotide which is operatively linked to a recombinase recognition site, the first exogenous polynucleotide eliciting a sterility-inducing phenotype in the PGC or in a gene-edited or genetically modified avian derived from the PGC, and stably integrating into the targeted GOI on the chromosome of interest in the PGC a second exogenous polynucleotide encoding a recombinase enzyme: wherein the first exogenous polynucleotide comprises a mutated or null GOI sequence or fragment thereof or encodes an endonuclease enzyme that can carry out genome editing; or wherein insertion of the first exogenous polynucleotide in the chromosome of interest modifies or disrupts the targeted GOI, the targeted GOI having: an isolated function specific to a PGC; or a function specific to gametogenesis, gamete maturation, or gamete function in the gene-edited or genetically modified avian; producing pure PGC colonies, the PGC colonies comprising the first exogenous polynucleotide and the second exogenous polynucleotide; transplanting a pure PGC colony to a male chick embryo to produce a chimera male chick embryo and transplanting a pure PGC colony to a female chick embryo to produce a chimera female chick embryo; hatching and rearing the chimera founder chick to sexual maturity as a chimera founder adult avian; screening the chimera founder adult avian to verify heterozygosity for the edited GOI; breeding a male chimera founder adult avian having heterozygosity for the edited GOI with a female chimera founder adult avian having heterozygosity for the edited GOI to produce progeny embryos; and identifying a sterile homozygotic embryo from the progeny embryos.

In some embodiments, the method further comprises providing a desired PGC having a desired trait of interest; and transplanting the desired PGC into the sterile homozygotic embryo.

In some embodiments, the modification or disruption of the targeted GOI has an isolated function specific to a PGC reduces or inhibits survival, maturation, or differentiation of a PGC derived from the gene-edited or genetically modified avian.

A skilled artisan would appreciate that the term “isolated function” may encompass a situation wherein if a GOI were knocked-out (KO), no other systems will be affected. This is a restriction that that is adopted as a measure of caution. i.e. one would not KO a gene that causes sterility if it causes blindness as well. Combining two GOI to maximize the effect of sterility, in case one is insufficient, means that the two genes have isolated function in the gametes. In certain embodiments, the term “isolated function” encompasses the function of a GOI that is only active in gametes.

In some embodiments, the modification or disruption of the targeted GOI has a function specific to gametogenesis, game maturation, or gamete function reduces or inhibits gametogenesis, meiosis, gamete function, or gamete fertilization in the gene-edited or genetically modified avian.

In some embodiments, the modification or disruption of the targeted GOI comprising modification or disruption of a gene encoding a protein selected from the group consisting of Zona Pellucida Binding Protein 1/2 (ZPBP1/2) protein, Cytokine-Dependent Kinases Regulatory Subunit 2 (CKS2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis-Specific With Coiled-Coil Domain-Containing Protein (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein. In some embodiments, the modification or disruption of the targeted GOI comprising modification or disruption of a gene encoding a Deleted In Azoospermia-Like (DAZL) protein.

In some embodiments, the modification or disruption of the targeted GOI comprising modification or disruption of more than one GOI in order to account for redundancy of mechanisms or shared functions. In some embodiments, the modification or disruption of at least 2 targeted GOIs comprises modification or disruption of at least 2 genes selected from those encoding a protein selected from the group consisting of Zona Pellucida Binding Protein 1/2 (ZPBP1/2) protein, Cytokine-Dependent Kinases Regulatory Subunit 2 (CKS2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis-Specific With Coiled-Coil Domain-Containing Protein (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein, or a combination thereof. In some embodiments, the modification or disruption comprises modification or disruption of at least 2, 3, 4, or 5 targeted GOIs.

In some embodiments, the chromosome of interest is an autosomal chromosome.

In some embodiments, the sterile phenotype induces sterility in a male gene-edited or genetically modified avian produced by the PGC and a female gene-edited or genetically modified avian produced by the PGC.

In some embodiments, the sequence for directing the first nucleic acid sequence or the second nucleic acid sequence to the chromosome of interest of the PGC comprises: a left homology arm (LHA) nucleotide sequence that is substantially homologous to the 5′ region flanking a target gene locus for the targeted GOI in the chromosome of interest of the PGC; and a right homology arm (RHA) nucleotide sequence that is substantially homologous to the 3′ region flanking the target gene locus for the targeted GOI in the chromosome of interest of the PGC.

In some embodiments, the first nucleic acid sequence or the second nucleic acid sequence comprises a detectable marker. In some embodiments, the marker is a fluorescent protein, a luminescent protein, or a chromoprotein. In some embodiments, the marker is (a) a fluorescent protein comprising Green Fluorescent Protein (GFP), Enhanced Green Fluorescent Protein (EGF), Emerald, Superfolder GFP, Azami Green, mWasabi, Tag-Green Fluorescent Protein (TagGFP), Turbo-Green Fluorescent Protein (TurboGFP), mNeonGreen, mUKG, acGFP, ZsGreen, Cloverm Sapphire, T-Sapphire, Enhanced Blue Fluorescent Protein (EBFP), Enhanced Blue Fluorescent Protein 2 (EBFP2), Azurite, Tag-Enhanced Blue Fluorescent Protein (TagBFP), mTagBFP, mKalamal, Cyan Fluorescent Protein (CFP), mCFP, Enhanced Cyan Fluorescent Protein (ECFP), mECFP, Cerulean, SCFP3A, mTurquoise, mTurquoise2, CyPet, AmCyan1, Midori-Ishi Cyan, Tag-Cyan Fluorescent Protein (TagCFP), mTFP1 (Teal), Yellow Fluorescent Protein (YFP), Enhanced Yellow Fluorescent Protein (EYFP), Super Yellow Fluorescent Protein (SYFP), Topaz, Venus, Citrine, mCitrine, YPet, Tag-Yellow Fluorescent Protein (TagYFP), Turbo-Yellow Fluorescent Protein (TurboYFP), Phi-Yellow Fluorescent Protein (PhiYFP), ZsYellow1, mBanana, Kusabira Orange, Kusabira Orange2, mOrange, mOrange2, dTomato, dTomato-Tandem, Red Fluorescent Protein (RFP), Turbo-Red Fluorescent Protein (TurboRFP), TurboFP602, TurboFP635, Tag-Red Fluorescent Protein (RFP), TagRFP-T, DsRed, DsRed2, DsRed-Express (T1), DsRed-Monomer, mTangerine, mKeima-Red, mRuby, mRuby2, mApple, mStrawberry, AsRed2, mRFP1, J-Red, mCherry, mKate (TagFP635), mKate2, HcRed1, mRaspberry, dKeima-Tandem, HcRed-Tandem, mPlum, mNeptune, NirFP, Sinus, TagFRP657, AQ143, Kaede, KikGR1, PX-CFP2, mEos2, IrisFP, mEOS3.2, PSmOrange, PAGFP, Dronpa, Allowphycocyanin, GFPuv, R-Phycoerythrin (RPE), Peridinin Chlorophyll (PerCP), P3, Katusha, B-Phycoerythrin (BPE), and mKO; or (b) a chromoprotein comprising ShadowR, Stichodactyla gigantea (sgBP), Heteractis crispa (hcCP), Anemonia sulcata (asCP), Cnidopus japonicus (cjBlue), or Goniopora tenuidens (gtCP). In some embodiments, the marker is mCherry.

In some embodiments, the first exogenous polynucleotide comprises any one of the sequences set forth in SEQ ID NO: 10, SEQ ID NO: 33, or SEQ ID NO: 34; and the second exogenous polynucleotide comprises sequences set forth in SEQ ID NO: 37.

In some embodiments, the PGC derived from an avian of the Galliformes order, the Anseriformes order, the Otidiformes order, the Columbiformes order, or the Struthioniformes order.

The method of claim 50, the PGC derived from an avian of the Galliformes order comprising the Phasianidae family or the Numididae family.

The method of claim 50, the PGC derived from an avian of the Anseriformes order comprising the Anatidae family, the Anseranatidae family, or the Anhimidae family.

The method of claim 50, the PGC derived from an avian of the Otidiformes order comprising the Otididae family.

The method of claim 50, the PGC derived from an avian of the Columbiformes order comprising the Columbidae family.

The method of claim 50, the PGC derived from an avian of the Struthioniformes order comprising the Struthionidae family.

### Avians

“Ayes,” “avians,” or “birds” are a class of warm-blooded vertebrates generally having feathers, forelimbs modified as wing, toothless beaked jaws, the laying of hard-shelled eggs, a high metabolic rate and body temperature, a four-chambered heart, and a lightweight skeleton often characterized by large air-filled cavities (pneumatic cavities) connecting with the respiratory system. In non-flightless birds and in most reluctant flyers, the sternum is keeled for the attachment of flight muscles. The avian immune system includes the bursa of Fabricius, which is unique to avians and which is the site of hematopoiesis and B cell development.

Avian reproduction takes place via cloacal kiss, and many female avians have sperm storage mechanisms enabling sperm from one or more males to remain viable within the female for a period of time following copulation. Following fertilization, the shell is applied to the egg, and the egg is then laid and incubated externally by one or both parents, by a non-parent male partner, or by a non-parent (even of another species, as in brood parasitism). The degree of monogamy depends on the species.

As used herein, the terms “ave,” “avian,” or “bird” refer to any avian species, including, but are not limited to, chicken, turkey, duck, goose, quail, pheasant, guinea fowl, pigeon, and ostrich. In certain embodiments, the bird is a species of fowl. In certain embodiments, the bird is a species of poultry. In certain embodiments, the bird is a domestic bird. In certain embodiments, the bird is Gallus gallus. In certain embodiments, the bird is a domestic Gallus gallus. In certain embodiments, the bird is Gallus gallus domesticus.

In certain embodiments, the bird is a female. In certain embodiments, the bird is a male. In certain embodiments, the bird is a broiler. In certain embodiments, the bird is a hen. In certain embodiments, the bird is layer hen. In certain embodiments, the bird is a domestic chicken. In certain embodiments, the bird is Gallus gallus domesticus layer hen.

“Domestication” by humans is a sustained multi-generational relationship in which humans significantly influence the reproduction and care of another species to secure a more predictable supply of, e.g., resources from the other species. Domestication is characterized by conscious selective breeding in which humans directly select for desirable traits, in contrast to unconscious selection in which traits evolve as a by-product of natural selection or from selection on other traits. As a result, there are genetic differences between wild and domesticated populations, even of the same species.

“Fowl” are primitive avians belonging to one of two orders—Galliformes (gamefowl or landfowl) and Anseriformes (waterfowl), which together form the fowl Glade Galloanserae. Galloanserae are noted for being very prolific, for having a high rate of polygamy compared with other avians, for hybridization and increased ability to interbreed (even where not closely related), and for precocious young.

“Poultry” are domesticated birds or birds that are captive-raised for meat, eggs, feathers, and the like. Most poultry belong to the Galloanserae Glade, but there are a few exceptions (e.g., ostriches). Popular examples of poultry include, but are not limited to, chicken, turkey, duck, goose, quail, pheasant, guinea fowl, pigeon, and ostrich.

Galliformes (gamefowl or landfowl) is an avian order characterized by a heavy body and ground-feeding and includes, but is not limited to, chicken, turkey, grouse, quail (both New World and Old World), ptarmigan, partridge, pheasant, francolin, junglefowl, and the Cracidae. The order comprises five families: Phasianidae (e.g., chicken, quail, partridge, pheasant, turkey, peafowl, grouse); Odontophoridae (e.g., New World quail); Numididae (e.g., guineafowl); Cracidae (e.g., chachalacas, curassows); and Megapodiidae (e.g., malleefowl, brush-turkey). The Phasianidae family includes, but is not limited to, the Gallus genus (e.g., Gallus gallus [wild or domestic chicken]) and the Meleagris genus (e.g., Meleagris gallopavo [wild or domestic turkey] or Meleagris ocellate [ocellated turkey]). The Numididae family of guineafowl includes the Agelastes, Numida, Guttera, and Acryllium genera.

Anseriformes (waterfowl) is an avian order characterized by aquatic lifestyle and swimming skills and includes, but is not limited to duck, goose, swan, magpie-goose, and screamer. The order comprises three families: Anhimidae (e.g., screamer); Anseranatidae (e.g., magpie-goose); and Anatidae (over 146 species in 43 genera, including e.g., duck, goose, swan). Unlike most birds, all except the screamers have phalli. The Anatidae family includes, but is not limited to, numerous species of ducks; three genera of gees (e.g., Answer, Branta, and Chen); and the Cygnus genus (e.g., Cygnus [swan]). The Anhimidae family includes, but is not limited to, the Anhima and Chauna genera.

Otidiformes (e.g., Otididae [bustards]) is an avian order characterized by large, terrestrial birds living mainly in dry grassland areas and on the steppes of the Old World.

They make up the family Otididae (formerly, Otidae or Gryzajidae). Bustards are omnivorous and opportunistic, eating leaves, buds, seeds, fruit, small vertebrates, and invertebrates. Twenty-six species currently recognized. Bustards include, but are not limited to, floricans and korhaans. The order comprises several families, including: Lissotis, Ardeotis, Neotis, Tetrax, Otis, Chlamydotis, Houbaropsis, Sypheotides, Lophotis, Eupodotis, and Afrotis. Chlamydotis (Houbara) is a genus of large birds in the bustard family.

Other avians used for meat, eggs, feathers, and the like include, but are not limited to members of the Columbiformes order (e.g., Columba Livia [pigeon]), the Struthioniformes order (e.g., Struthionidae camelus [ostrich]).

As used herein, the term “egg” refers to an avian egg that contains a viable or a live embryonic bird. In one embodiment, the term “egg” is intended to refer to a fertilized avian egg. In one embodiment, an egg is an egg containing an avian embryo that is capable of undergoing normal embryogenesis.

### Primordial Germ Cells (PGCs)

An “anlage” or “primordium” is an organ or tissue in its earliest recognizable stage of development or the simplest set of cells capable of triggering growth of the would-be organ or tissue and the initial foundation from which an organ or tissue is able to grow. A skilled artisan would appreciate that the terms “pluripotent cells” and “totipotent cells” of the primordium encompass “primordial cells.”

A “primordial germ cell” (PGC), also known as a “precursor germ cell” or a “gonocyte,” is a pluripotent diploid germ cell prior to its maturation as a gamete and is one of a small group of cells set aside during embryonic gastrulation to eventually form an oocyte or a spermatozoon. In birds and mammals, germ cells are formed during development in response to signals controlled by zygotic genes. In birds and reptiles, the PGC comes from the epiblast and migrates to the hypoblast to form the germinal crescent (an anterior extraembryonic structure). The PGC then enters into a blood vessel and uses the circulatory system for transport to a gonadal ridge (genital ridge), where it exits the blood vessel and enters the gonad. During its migration, it will divide repeatedly. Upon reaching the gonad, it becomes a “germ cell.” A germ cell gives rise to one or more “gametes” (ovum or sperm) and is the only type of cell capable of both meiosis and mitosis.

“Gametogenesis” refers to, e.g., the development of a diploid germ cell into a haploid ovum (oocyte, egg) or sperm (“oogenesis” or “spermatogenesis,” respectively). The process, especially oogenesis, may be arrested for a period of time prior to full “gamete maturation.” Once matured, a haploid male gamete and a haploid female gamete have the ability to unite to form a new, diploid cell (“zygote”).

PGCs, germ cells and gametes each have many unique properties. Two examples of features that make PGCs unique are their ability to maintain totipotency and differentiate into functional gametes, and their ability to migrate along the route from their initial location of formation to the gonads. With respect to migration, some genes involved in this process may also be involved in other functions in somatic cells. With respect to totipotency and gametogenesis, there are several genes active in these processes, but only a few of them are known to be expressed solely in PGCs without having a redundant alternative gene (e.g., Deleted in AZoospermia-Like [DAZL]).

Modification of a gene having an isolated function specific to a PGC includes, but is not limited to, reduction or inhibition of PGC survival, maturation, or differentiation or a combination thereof of a PGC derived from a gene-edited or genetically modified avian.

Modification of a gene having a function specific to gametogenesis, gamete maturation, or gamete function includes, but is not limited to, reduction or inhibition or a combination thereof, of gametogenesis, meiosis, gamete function, or gamete fertilization in the gene-edited or genetically modified avian.

For example, at sexual maturity of the organism, the primary oocyte secretes proteins to form a coat called zona pellucida and also produces cortical granules containing enzymes and proteins needed for fertilization. In addition, large non-mammalian oocytes accumulate egg yolk, glycogen, lipids, ribosomes, and the mRNA needed for protein synthesis during embryonic growth. In another example, the sperm cell undergoes nuclear condensation, ejection of the cytoplasm, and formation of the acrosome and flagellum.

### Target Genes for Sterility

Several genes are associated with sterility in basic research studies, as well as in clinical medicine. These can be broadly divided into two groups: the first, genes which are associated with sterility and also function in somatic cells, and the second, are genes with isolated function in PGCs, gametes maturation or gametes function. The latter group are targeted to induce embryonic sterility while leaving the organism otherwise healthy. These include, but are not limited to, zona pellucida binding protein 1/2 (ZPBP1/2) gene, Cyclin-dependent kinases regulatory subunit 2 (CKS2; CDC28 Protein Kinase Regulatory Subunit 2) gene, spermatogenesis associated 16 (SPATA16) gene, DEAD-box helicase 4 (DDX4) gene, serine/threonine-protein phosphatase PP1-gamma catalytic subunit (PPP1CC) gene, Izumo sperm-egg fusion 1 (IZUMO1) gene, synaptonemal complex central element protein 1 (SYCE1) gene, YTH domain-containing 2 (YTHDC2) gene, Meiosis Specific With Coiled-Coil Domain (MEIOC) gene, septin-4 (SEPT4) gene, Stromal antigen 3 (STAG3) gene, Nanos C2HC-type zinc finger 3 (NANOS3) gene, deleted in azoospermia 1 (DAZ1) gene, deleted in azoospermia-like (DAZL) gene and many more. Collectively, these genes each have an isolated role in one or more aspects of PGC survival, maturation or differentiation in one or more aspects of gametogenesis, meiosis, gametes function or fertilization. In certain cases, the proteins encoded by these genes may have a redundancy of function or mechanism. Thus, in some instances, it may be advantageous to mutate at least two genes in order to reduce or eliminate a specific function, e.g., functions associated with sterility, gametes maturation, or gametes function or a combination thereof. From these genes, the ones associated with both sexes' sterility, located on autosomal chromosomes and having well annotated evolutionary-conserved orthologous sequences in chickens or other avians, are advantageous.

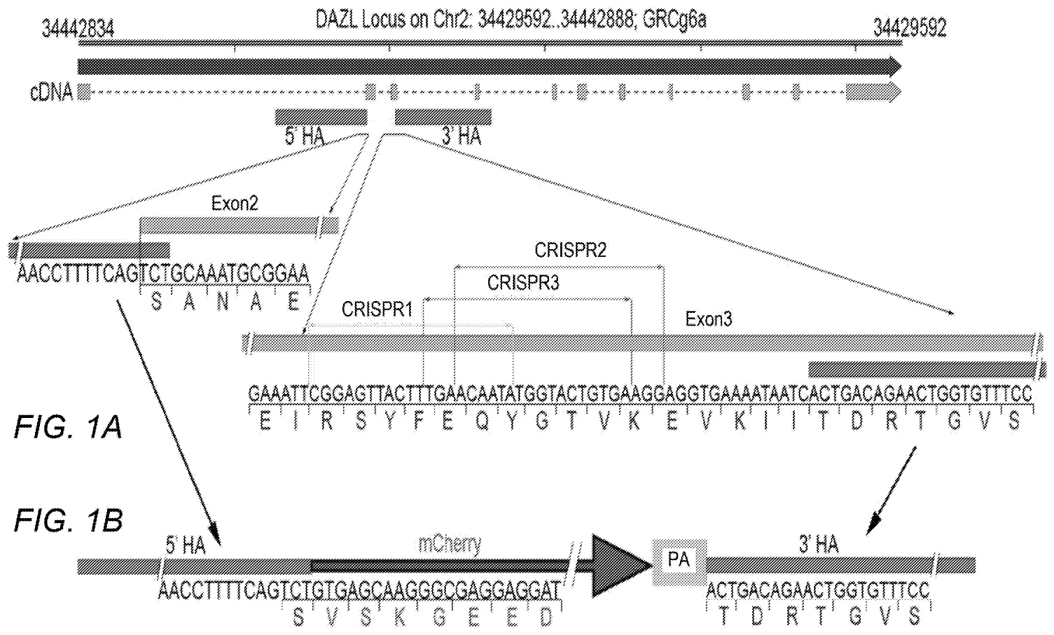

Notably, in some embodiments, the RNA binding protein Deleted in AZoospermia-Like (DAZL) is a key determinant of germ cell maturation and entry into meiosis in many species. Highly conserved in evolution the chicken DAZL (Smorag et al. 2014. Wiley Interdiscip. Rev.: RNA 5: 527-535), located on chromosome 2 in a well annotated region (Chr2: 34429592 . . . 34442834; GRCg6a; see FIG. 1A, which depicts a chromosomal map of DAZL). In accordance with its roles, DAZL is expressed in PGCs in both male and female embryos, thus it serves as marker for PGCs population. DAZL is a member of the Daz family genes, which share conserved evolutionary role in numerous model-organisms, including Caenorhabditis elegans, Drosophila melanogaster fly, fish, frog, mice and human patients (Fu et al. [2015] Intl. J. Biol. Sci. 11: 1226-1235), that result in sterility due to PGCs failure to form or mature to a functional adult gametes. Clearly, by definition, sterile chickens cannot breed, therefore healthy and fertile heterozygotes, which carry a single mutated allele, are required to preserve the trait, and by crossing heterozygotes, DAZL null sterile embryos are obtained. Where the mutation or ablation of the gene does not have an impact on viability, the heterozygous-heterozygous cross will produce homozygous DAZL null sterile embryos in a Mendelian ratio of 1:4.

Examples of genes having an isolated function specific to a PGC or genes having a function specific to gametogenesis, gamete maturation, or gamete function include, but are not limited to, zona pellucida binding protein 1/2 (ZPBP1/2) gene, cyclin-dependent kinases regulatory subunit 2 (CKS2; CDC28 Protein Kinase Regulatory Subunit 2) gene, spermatogenesis associated 16 (SPATA16) gene, DEAD-box helicase 4 (DDX4) gene, serine/threonine-protein phosphatase PP1-gamma catalytic subunit (PPP1CC) gene, Izumo sperm-egg fusion 1 (IZUMO1) gene, synaptonemal complex central element protein 1 (SYCE1) gene, YTH domain-containing 2 (YTHDC2) gene, meiosis specific with coiled-coil domain (MEIOC) gene, septin-4 (SEPT4) gene, stromal antigen 3 (STAG3) gene, Nanos C2HC-type zinc finger 3 (NANOS3) gene, deleted in azoospermia 1 (DAZ1) gene, deleted in azoospermia-like (DAZL) gene, respectively encoding Zona Pellucida Binding Protein 1/2 (ZPBP1/2) protein, Cytokine-Dependent Kinases Regulatory Subunit 2 (CKS2; CDC28 Protein Kinase Regulatory Subunit 2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis Specific With Coiled-Coil Domain (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein. While these genes may encode proteins comprising isolated functions, for example those specific to a PGC or specific to gametogenesis, gamete maturation, or gamete function, a skilled artisan would appreciate that in some embodiments, proteins may have redundant mechanisms or a shared function.

In some embodiments, the chromosome of interest is an autosomal chromosome. In some embodiments, the sterile phenotype induces sterility in a male gene-edited or genetically modified avian produced by the PGC and a female gene-edited or genetically modified avian produced by the PGC.

In some embodiments, the sterile phenotype induces sterility in a male gene-edited avian produced by the PGC and a female gene-edited avian produced by the PGC. In some embodiments, the sterile phenotype induces sterility in a mail gene knockout avian produced by the PGC and a female gene knockout produced by the PGC.

In some embodiments, the sterile phenotype induces sterility in a male genetically modified avian produced by the PGC and a female genetically modified avian produced by the PGC. In some embodiments, the sterile phenotype induces sterility in a male transgenic avian produced by the PGC and a female transgenic avian produced by the PGC.

### Genome Editing

Genome editing using engineered endonucleases refers to a genetic method using nucleases to cut and create specific double-stranded breaks at a desired location(s) in the genome (e.g. on the chromosome of interest of a bird), which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize only a few base pairs on the DNA as their target and the probability is very high that the recognized base pair combination will be found in many locations across the genome, resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and the CRISPR/Cas system.

Meganucleases—They are commonly grouped into four families: the LAGLIDADG family, the GIY-YIG family, the His-Cys box family and the HNH family. These families are characterized by structural motifs that affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp), thus making them naturally very specific for cutting at a desired location. This can be exploited to make site-specific double-stranded breaks in genome editing. One of skilled in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (e.g. U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514.

ZFNs and TALENs—Two distinct classes of engineered nucleases, zinc-finger nucleases (ZFNs) and transcription activator-like effector nucleases (TALENs), have both been proven to be effective at producing targeted double-stranded breaks. Basically, ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separated from each other is selected. The cleaving portion is separated and then linked to a DNA binding domain, thereby yielding an endonuclease with very high specificity for a desired sequence. An exemplary restriction enzyme with such properties is Fokl. Additionally, Fold has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example, ZFNs and TALENs can be constructed as nuclease pairs, with each member of the pair designed to bind adjacent sequences at the targeted site. Upon transient expression in cells, the nucleases bind to their target sites and the Fold domains heterodimerize to create a double-stranded break. Repair of these double-stranded breaks through the non-homologous end-joining (NHEJ) pathway most often results in INDELs which are small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (see, e.g., Carlson et al., 2012, Proc Natl Acad Sci USA.; 109(43):17382-7; Lee et al., 2010, Trends Biotechnol.; 28(9):445-6). In addition, when a fragment of DNA with homology to the targeted region is introduced in conjunction with the nuclease pair, the double-stranded break can be repaired via homology directed repair to generate specific modifications (see, e.g., Li et al., 2011, Nucleic Acids Res. 39(1):359-72; Miller et al., 2010, Nat Struct Mol Biol. 17(9):1144-51; Urnov et al., 2005, Nature 435(7042):646-51).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others.

CRISPR-Cas system—Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (e.g. Cho et al., 2013, Nat Biotechnol. 31(3):230-2; Cong et al., 2013, Science 339(6121):819-23; DiCarlo et al., 2013, Nucleic Acids Res. 41(7):4336-43; Hwang et al., 2013, Nat Biotechnol. 31(3):227-9; Jinek et al., 2013, Elife. 2013 Jan. 29; 2:e00471; Mali et al., 2013, Nat Methods. 10(10):957-63).

It is known that the CRIPSR/Cas system for genome editing contains two distinct components: a guide RNA (gRNA) and an endonuclease e.g. Cas9. The gRNA is typically a 20-nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded breaks produced by CRISPR/Cas can undergo homologous recombination or NHEJ. In certain embodiments, the CRISPR/Cas system comprises single guide RNA (sgRNA) and a Cas protein. In certain embodiments, the CRISPR/Cas system comprises a complex of single guide RNA (sgRNA) and a Cas protein. In certain embodiments, the Cas of the CRISPR/Cas system comprises a single polypeptide. In certain embodiments, the Cas of the CRISPR/Cas system is an endonuclease. In certain embodiments, the CRISPR/Cas is CRISPR/Cas9.

The Cas9 nuclease has two functional domains: RuvC and HNH, each cutting a different DNA strand. When both domains are active, the Cas9 causes double strand breaks in the genomic DNA. A significant advantage of CRISPR/Cas is that the high efficiency of this system coupled with the ability to easily create synthetic gRNAs enables multiple genes to be targeted simultaneously. Apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription. In certain embodiments, the CRISPR/Cas is CRISPR/dCas9.

In order to use the CRISPR system, both gRNA and Cas9 should be expressed in a target cell. The insertion vector can contain both cassettes on a single plasmid or the cassettes are expressed from two separate plasmids. CRISPR plasmids are publicly available such as the px330 plasmid from Addgene. Additionally, mRNA encoding Cas9 and the gRNA can be introduced to the target cells as well as recombinant Cas9 protein in complex with the gRNA (i.e. insert the RNP complex into the cell).

In certain embodiments, the CRISPR/Cas system is a Class 1 CRISPR/Cas system. In certain embodiments, a Class 1 CRISPR/Cas system comprises a multi-subunit crRNA—effector complex. In certain embodiments, the CRISPR/Cas system is a type I CRISPR—Cas system. In certain embodiments, the CRISPR/Cas system is a type III CRISPR/Cas system. In certain embodiments, the CRISPR/Cas system is a type IV CRISPR—Cas system.

In certain embodiments, the CRISPR/Cas system is a Class 2 CRISPR/Cas system. In certain embodiments, a Class 2 CRISPR/Cas system comprises a single subunit crRNA—effector module. In certain embodiments, the CRISPR/Cas system is a type II CRISPR—Cas system. In certain embodiments, the CRISPR/Cas system is a type V CRISPR/Cas system.

In certain embodiments, the Cas in the Class 2 CRISPR/Cas system can be Cas9, Cpf1, C2c1, C2c2 or C2c3. A person of ordinary skill in the art would understand the classification of CRISPR/Cas systems as it is well-known in the art (e.g., Nat Rev Microbiol. 2017 Mar., 15(3): 169-182; Nat Rev Microbiol. 2015 Nov., 13(11): 722-736), and that this classification is evolving with time (Mol Cell. 2015 Nov. 5, 60(3): 385-397). In some embodiments, the CRISPR/Cas is any CRISPR associated protein (CAS) endonuclease known in the art.

Genome editing using recombinant adeno-associated viruses (rAAVs) is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross or subtle endogenous gene alterations in a cell or a combination thereof rAAV genome editing has the advantage in that it targets a single allele and does not result in any off-target genomic alterations.

### DNA Editing Agent

The technology described herein provides, in certain aspects and embodiments, a DNA-editing agent. The DNA editing agent may be constructed using recombinant DNA technology well known to persons skilled in the art.

In one embodiment, the DNA editing agent disclosed herein may be comprised in a single nucleic acid construct, or comprised in a combination of nucleic acid constructs. In one embodiment, the DNA editing agent comprises at least two key elements as described below:

A first agent comprising a first nucleic acid sequence for eliciting a sterile phenotype in a gene-edited or genetically modified avian operatively linked to a recombinase recognition site and a sequence for directing the first nucleic acid sequence for effecting the sterile phenotype to a targeted gene of interest (GOI) on a chromosome of interest of a PGC. In some embodiments, the first nucleic acid sequence encodes a sterility-inducing protein or an endonuclease enzyme that can carry out genome editing. In some embodiments, insertion of the first nucleic acid sequence in the chromosome of interest modifies or disrupts the GOI, e.g., where the GOI has an isolated function specific to a PGC (e.g., reducing or inhibiting survival, maturation, or differentiation of a PGC derived from a gene-edited or genetically modified avian derived from the DNA edited PGC), or where the GOI has a function specific to gametogenesis, gamete maturation, or gamete function in the gene-edited or genetically modified avian (e.g., reducing or inhibiting gametogenesis, meiosis, gamete function, or gamete fertilization in the gene-edited or genetically modified avian) or a combination thereof. Examples of target genes for sterility are described elsewhere herein. In some embodiments, the chromosome of interest is an autosomal chromosome. In some embodiments, the sterile phenotype induces sterility in a male gene-edited or genetically modified avian produced by the PGC and a female gene-edited or genetically modified avian produced by the PGC.

A second agent comprising a second nucleic acid sequence, the second nucleic acid sequence encoding a recombinase enzyme and a sequence for directing the second nucleic acid sequence to the targeted GOI on the chromosome of interest of the PGC.

In some embodiments, the sequence for directing the first nucleic acid sequence or the second nucleic acid sequence or a combination thereof, to the chromosome of interest of the PGC comprises a left homology arm (LHA) nucleotide sequence that is substantially homologous to the 5′ region flanking a target gene locus in the chromosome of interest of the PGC; and a right homology arm (RHA) nucleotide sequence that is substantially homologous to the 3′ region flanking the target gene locus in the chromosome of interest of the PGC.

A person of skill in the art would understand that the term “DNA editing agent” generally refers to any molecule, such as a nucleotide sequence or an enzyme, which promotes a change in a genome of an organism, such as a bird. The change may be an addition to the DNA, e.g. by the agent being integrated to the DNA, a replacement of a sequence of the DNA, e.g. by homological recombination, or a deletion of the DNA.

In one embodiment, the DNA editing agent may be constructed in a viral vector (e.g. using a single vector or multiple vectors). Such vectors are commonly used in gene transfer and gene therapy applications. Different viral vector systems have their own unique advantages and disadvantages. Viral vectors that may be used to integrate the first nucleotide sequence of certain embodiments into the chromosome of interest of a bird include, but are not limited to, adenoviral vectors, adeno-associated viral vectors, alphavirus vectors, herpes simplex viral vectors, retroviral vectors, or lentiviral vectors.

A viral construct such as a retroviral construct includes at least one transcriptional promoter/enhancer or locus-defining element(s), or other elements that control gene expression by means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. In certain embodiments, the signal sequence can be a mammalian signal sequence. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, or dendrimers.

In certain embodiments, the DNA editing agent comprises the sequence of one of SEQ ID NOs: 10, 33, or 34.

The DNA editing agent may encode a reporter protein that is readily detectable either by its presence or activity, including, but not limited to, luciferase, fluorescent protein (e.g., green fluorescent protein), chloramphenicol acetyl transferase, beta-galactosidase, secreted placental alkaline phosphatase, beta-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and typically without the need to kill the cells for signal analysis. In certain embodiments, a reporter gene encodes an enzyme, which produces a change in fluorometric properties of the host cell, which is detectable by qualitative, quantitative, or semi-quantitative function or transcriptional activation. Exemplary enzymes include esterases, β-lactamase, phosphatases, peroxidases, proteases (tissue plasminogen activator or urokinase) and other enzymes whose function can be detected by appropriate chromogenic or fluorogenic substrates known to those skilled in the art or developed in the future. The reporter gene may report on successful integration of the construct into the chromosome of interest.

In certain embodiments, the DNA editing agent may comprise a nucleotide sequence that encodes a detectable marker (e.g., a reporter polypeptide).

In certain embodiments, the DNA editing agent further comprises a positive or a negative selection marker, or a combination thereof, for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations or elimination of a marker sequence (e.g. positive marker), or combinations thereof. Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT), Diphtheria toxin (DT) and adenine phosphoribosyltransferase (ARPT).

In certain embodiments, the codons encoding the proteins of the DNA editing agent are “optimized” codons, i.e., the codons are those that appear frequently in, e.g., highly expressed genes in the bird species, instead of those codons that are frequently used by, for example, an influenza virus. Such codon usage provides for efficient expression of the protein in avian cells. Codon usage patterns are known in the literature for highly expressed genes of many species (e.g., Nakamura et al., 1996, Nucleic Acids Res. 24(1):214-5; McEwan et al., 1998, Biotechniques. 24(1):131-6, 138).

In certain embodiments, the DNA editing agent may further include self-cleaving peptides such as the 2A, including but not limited to P2A, T2A, E2A (Wang et al., Scientific Report 5, Article 16273 (2015)), or internal ribosome entry site (IRES) sequences.

### Left and Right Homology Arms

In certain embodiments, (i) the length of the LHA is about 0.5 to about 5 kilobases (kb); (ii) the length of the RHA is about 0.5 to about 5 kb; or (iii) any combination of (i) and (ii). In certain embodiments, (i) the length of the LHA is about 1.5 kb; (ii) the length of the RHA is about 1.5 kb; or (iii) any combination of (i) and (ii).

In certain embodiments, the length of the LHA is about 0.5 to about 5 kilobases (kb). In certain embodiments, the length of the LHA is about 0.5 kb. In certain embodiments, the length of the LHA is about 1 kb. In certain embodiments, the length of the LHA is about 1.5 kb. In certain embodiments, the length of the LHA is about 2 kb. In certain embodiments, the length of the LHA is about 2.5 kb. In certain embodiments, the length of the LHA is about 3 kb. In certain embodiments, the length of the LHA is about 3.5 kb. In certain embodiments, the length of the LHA is about 4 kb. In certain embodiments, the length of the LHA is about 4.5 kb. In certain embodiments, the length of the LHA is about 5 kb.

In certain embodiments, the length of the RHA is about 0.5 to about 5 kilobases (kb). In certain embodiments, the length of the RHA is about 0.5 kb. In certain embodiments, the length of the RHA is about 1 kb. In certain embodiments, the length of the RHA is about 1.5 kb. In certain embodiments, the length of the RHA is about 2 kb. In certain embodiments, the length of the RHA is about 2.5 kb. In certain embodiments, the length of the RHA is about 3 kb. In certain embodiments, the length of the RHA is about 3.5 kb. In certain embodiments, the length of the RHA is about 4 kb. In certain embodiments, the length of the RHA is about 4.5 kb. In certain embodiments, the length of the RHA is about 5 kb.

In certain embodiments, the length of each of the LHA and RHA is about 0.5 kb. In certain embodiments, the length of each of the LHA and RHA is about 1 kb. In certain embodiments, the length of each of the LHA and RHA is about 1.5 kb. In certain embodiments, the length of each of the LHA and RHA is about 2 kb. In certain embodiments, the length of each of the LHA and RHA is about 2.5 kb. In certain embodiments, the length of each of the LHA and RHA is about 3 kb. In certain embodiments, the length of each of the LHA and RHA is about 3.5 kb. In certain embodiments, the length of each of the LHA and RHA is about 4 kb. In certain embodiments, the length of each of the LHA and RHA is about 4.5 kb. In certain embodiments, the length of each of the LHA and RHA is about 5 kb.

In certain embodiments, the length of each of the left and the right homology arms is sufficient to allow specific recombination into chromosomal DNA of a bird. In one embodiment, the LHA or the RHA or a combination thereof, are at least 500 nucleotides long, for example, between 500-3000 nucleotides long. Typically, the required size of the LHA or the RHA or both homology arms relies on the length of the cassettes which are flanked by these arms. Smaller cassettes require shorter arms and vice versa.

In certain embodiments, (i) the LHA is substantially homologous to a corresponding first nucleotide sequence located in an openly transcribed region on the chromosome of interest of a bird; (ii) the RHA is substantially homologous to a corresponding second nucleotide sequence located in an openly transcribed region on the chromosome of interest of a bird; or (iii) both (i) and (ii).

As it would be apparent to those skilled in the art, a first sequence is “substantially homologous” to a second sequence if the first sequence and the second sequence are similar or identical in sequence, as long as the first sequence and the second sequence can replace one another by homologous recombination. Method to test and identify homologous recombination are well-known in the art.

In certain embodiments, substantially homologous is at least 50% identical. In certain embodiments, substantially homologous is at least 60% identical. In certain embodiments, substantially homologous is at least 70% identical. In certain embodiments, substantially homologous is at least 80% identical. In certain embodiments, substantially homologous is at least 90% identical. In certain embodiments, substantially homologous is at least 95% identical. In certain embodiments, substantially homologous is at least 99% identical.

In certain embodiments, the first nucleotide sequence in the LHA is 50% to 100% identical in sequence to a first corresponding nucleotide sequence on the chromosome of interest. In certain embodiments, the first nucleotide sequence in the LHA is 80% to 100% identical in sequence to a first corresponding nucleotide sequence on the chromosome of interest. In certain embodiments, the first nucleotide sequence in the LHA is 85% to 100% identical in sequence to a first corresponding nucleotide sequence on the chromosome of interest. In certain embodiments, the first nucleotide sequence in the LHA is 90% to 100% identical in sequence to a first corresponding nucleotide sequence on the chromosome of interest. In certain embodiments, the first nucleotide sequence in the LHA is 95% to 100% identical in sequence to a first corresponding nucleotide sequence on the chromosome of interest. In certain embodiments, the first nucleotide sequence in the LHA is 99% to 100% identical in sequence to a first corresponding nucleotide sequence on the chromosome of interest. In certain embodiments, the first nucleotide sequence in the LHA is 100% identical in sequence to a first corresponding nucleotide sequence on the chromosome of interest.

In certain embodiments, the fourth nucleotide sequence in the RHA is 50% to 100% identical in sequence to a second corresponding nucleotide sequence on the chromosome of interest. In certain embodiments, the fourth nucleotide sequence in the RHA is 80% to 100% identical in sequence to a second corresponding nucleotide sequence on the chromosome of interest. In certain embodiments, the fourth nucleotide sequence in the RHA is 85% to 100% identical in sequence to a second corresponding nucleotide sequence on the chromosome of interest. In certain embodiments, the fourth nucleotide sequence in the RHA is 90% to 100% identical in sequence to a second corresponding nucleotide sequence on the chromosome of interest. In certain embodiments, the fourth nucleotide sequence in the RHA is 95% to 100% identical in sequence to a second corresponding nucleotide sequence on the chromosome of interest. In certain embodiments, the fourth nucleotide sequence in the RHA is 99% to 100% identical in sequence to a second corresponding nucleotide sequence on the chromosome of interest. In certain embodiments, the fourth nucleotide sequence in the RHA is 100% identical in sequence to a second corresponding nucleotide sequence on the chromosome of interest.

In certain embodiments, the LHA or the RHA or bother, are homologous or show homology or identity of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to at least one nucleotide sequence within a target loci within the chromosome of interest of a bird that serves as the integration site.

A person of skill in the art would understand that the term “openly transcribed region in a chromosome” generally refers to regions of the chromosome which include genes that are transcribed in a level sufficient to allow other genes to be easily transcribed as well. Non-limiting examples of openly transcribed regions are regions in proximity to house-keeping genes which are highly transcribed during the life of the cell or the organism. Other non-limiting examples of openly transcribed regions are regions in-between loci (e.g. chromatin regulatory elements, non-coding DNA, “junk DNA”, etc.). Non-limiting examples of poorly transcribed regions are regions at the ends of each chromosomes, called telomers, which are not transcribed during the life of the cell or the organism.

In certain embodiments, the openly transcribed region is located on chromosome 2 of a bird. In certain embodiments, the openly transcribed region is located on chromosome 2 of Gallus gallus.

In one embodiment, the LHA or the RHA or both correspond to a genomic sequence which is present on chromosome 2 in birds.

The LHA or the RHA targeting sequences or both, may be selected such that the LHA or the RHA targeting sequence or both integrate specifically into the chromosome of interest and not any other chromosome of the cell, e.g. by spontaneous homologous recombination or by homology directed repair (HDR). Homologous recombination can occur spontaneously. Furthermore, the LHA or the RHA targeting sequence or both may be selected depending on what method is being relied upon to integrate the first targeting sequence into the chromosome. Methods of integrating nucleotide sequences into chromosomes are well known in the art, including targeted homologous recombination, site specific recombinases and genome editing by engineered nucleases (see e.g. Menke D. Genesis (2013) 51:-618; Capecchi, Science (1989) 244:1288-1292; Santiago et al., Proc Natl Acad Sci USA (2008) 105:5809-5814; International Patent Application Nos. WO 2014/085593, WO 2009/071334 and WO 2011/146121; U.S. Pat. Nos. 8,771,945, 8,586,526, 6,774,279 and US Patent Application Publication Nos. 2003/0232410, 2005/0026157, 2006/0014264). PB transposases are also contemplated. Agents for introducing nucleic acid alterations to a gene of interest can be designed by publicly available resources. In certain embodiments, the first 5′ nucleotide of LHA corresponds to chromosome 2, position 34439663. In certain embodiments, the first corresponding nucleotide sequence of LHA is located at chromosome 2, position 34439663 to position 34438203.

In certain embodiments, the first 5′ nucleotide of LHA corresponds to Gallus gallus chromosome 2, Assembly GRCg6a, NC_006089.5https://www.ncbi.nlm.nih.gov/nucleotideNC_006089.5?report=genbank&lo g$=nuclalign&blast_rank=1&RID=HDGPY1G3014, position 34439663. In certain embodiments, the first corresponding nucleotide sequence of LHA is located at Gallus gallus chromosome 2, Assembly GRCg6a, NC 006089.5, position 34439663 to position 34438203.

In certain embodiments, the first 5′ nucleotide of RHA corresponds to chromosome 2, position 34437739. In certain embodiments, the second corresponding nucleotide sequence of RHA is located at chromosome 2, position 34437739 to position 34436209.

In certain embodiments, the first 5′ nucleotide of RHA corresponds to Gallus gallus chromosome 2, Assembly GRCg6a, NC 006089.5, position 34437739. In certain embodiments, the second corresponding nucleotide sequence of RHA is located at Gallus gallus chromosome 2, Assembly GRCg6a, NC 006089.5, position 34437739 to position 34436209.

### Detectable Markers

Genetic modification of the avian PGC chromosome may include a detectable marker that is detectable, e.g., in the PGC, in a gene-edited or genetically modified avian produced by the PGC, or in a PGC produced by the gene-edited or genetically modified avian produced by the PGC. In some embodiments, the detectable marker comprises a reporter polypeptide. Examples of a “detectable marker” include, but are not limited to, a fluorescent protein, a luminescent protein, a chromoprotein, an audible (vibrating) protein, a sonic protein, a metabolic marker, or a selective chelating protein. Examples of a fluorescent protein include, but are not limited to, green fluorescent protein (GFP), enhanced green fluorescent protein (EGF), Emerald, Superfolder GFP, Azami Green, mWasabi, TagGFP, TurboGFP, mNeonGreen, mUKG, acGFP, ZsGreen, Cloverm Sapphire, T-Sapphire, Enhanced blue fluorescent protein (EBFP), EBFP2, Azurite, TagBFP, mTagBFP, mKalamal, Cyan fluorescent protein (CFP), mCFP, Enhanced cyan fluorescent protein (ECFP), mECFP, Cerulean, SCFP3A, mTurquoise, mTurquoise2, CyPet, AmCyan1, Midori-Ishi Cyan, TagCFP, mTFP1 (Teal), Yellow fluorescent protein (YFP), Enhanced yellow fluorescent protein (EYFP), Super yellow fluorescent protein (SYFP), Topaz, Venus, Citrine, mCitrine, YPet, TagYFP, TurboYFP, PhiYFP, ZsYellow1, mBanana, Kusabira Orange, Kusabira Orange2, mOrange, mOrange2, dTomato, dTomato-Tandem, Red fluorescent protein (RFP), TurboRFP, TurboFP602, TurboFP635, Tag ref fluorescent protein (RFP), TagRFP-T, DsRed, DsRed2, DsRed-Express (T1), DsRed-Monomer, mTangerine, mKeima-Red, mRuby, mRuby2, mApple, mStrawberry, AsRed2, mRFP1, J-Red, mCherry, mKate (TagFP635), mKate2, HcRed1, mRaspberry, dKeima-Tandem, HcRed-Tandem, mPlum, mNeptune, NirFP, Sinus, TagFRP657, AQ143, Kaede, KikGR1, PX-CFP2, mEos2, IrisFP, mEOS3.2, PSmOrange, PAGFP, Dronpa, Allowphycocyanin, GFPuv, R-phycoerythrin (RPE), Peridinin Chlorophyll (PerCP), P3, Katusha, B-phycoerythrin (BPE), and mKO, as well as derivatives and combinations thereof. In some embodiments, the marker is mCherry.

Examples of chromoproteins include, but are not limited to, or (b) a chromoprotein comprising ShadowR, Stichodactyla gigantea (sgBP), Heteractis crispa (hcCP), Anemonia sulcata (asCP), Cnidopus japonicus (cjBlue), or Goniopora tenuidens (gtCP), as well as derivatives and combinations thereof.

In certain embodiments, the detectable marker can be a green fluorescence protein (GFP) (SEQ ID NO: 23), or mCherry/RFP (SEQ ID NO: 24), as shown in Table 1.

TABLE 1
Examples of marker sequences.

| Marker | Sequence |
|---|---|
| GFP | ATGGTGAGCAAGGGCGAGGAGCTGTTCACCGGGGTGGTG |
| | CCCATCCTGGTCGAGCTGGACGGCGACGTAAACGGCCAC |
| | AAGTTCAGCGTGTCCGGCGAGGGCGAGGGCGATGCCACC |
| | TACGGCAAGCTGACCCTGAAGTTCATCTGCACCACCGGCA |
| | AGCTGCCCGTGCCCTGGCCCACCCTCGTGACCACCCTGAC |
| | CTACGGCGTGCAGTGCTTCAGCCGCTACCCCGACCACATG |
| | AAGCAGCACGACTTCTTCAAGTCCGCCATGCCCGAAGGCT |
| | ACGTCCAGGAGCGCACCATCTTCTTCAAGGACGACGGCA |
| | ACTACAAGACCCGCGCCGAGGTGAAGTTCGAGGGCGACA |
| | CCCTGGTGAACCGCATCGAGCTGAAGGGCATCGACTTCA |
| | AGGAGGACGGCAACATCCTGGGGCACAAGCTGGAGTACA |
| | ACTACAACAGCCACAACGTCTATATCATGGCCGACAAGC |
| | AGAAGAACGGCATCAAGGTGAACTTCAAGATCCGCCACA |
| | ACATCGAGGACGGCAGCGTGCAGCTCGCCGACCACTACC |
| | AGCAGAACACCCCCATCGGCGACGGCCCCGTGCTGCTGC |
| | CCGACAACCACTACCTGAGCACCCAGTCCGCCCTGAGCA |
| | AAGACCCCAACGAGAAGCGCGATCACATGGTCCTGCTGG |
| | AGTTCGTGACCGCCGCCGGGATCACTCTCGGCATGGACG |
| | AGCTGTACAAGTAA (SEQ ID NO: 23) |
| mCherry | ATGGTGAGCAAGGGCGAGGAGGATAACATGGCCATCATC |
| | AAGGAGTTCATGCGCTTCAAGGTGCACATGGAGGGCTCC |
| | GTGAACGGCCACGAGTTCGAGATCGAGGGCGAGGGCGAG |
| | GGCCGCCCCTACGAGGGCACCCAGACCGCCAAGCTGAAG |
| | GTGACCAAGGGTGGCCCCCTGCCCTTCGCCTGGGACATCC |
| | TGTCCCCTCAGTTCATGTACGGCTCCAAGGCCTACGTGAA |
| | GCACCCCGCCGACATCCCCGACTACTTGAAGCTGTCCTTC |
| | CCCGAGGGCTTCAAGTGGGAGCGCGTGATGAACTTCGAG |
| | GACGGCGGCGTGGTGACCGTGACCCAGGACTCCTCCCTG |
| | CAGGACGGCGAGTTCATCTACAAGGTGAAGCTGCGCGGC |
| | ACCAACTTCCCCTCCGACGGCCCCGTAATGCAGAAGAAG |
| | ACCATGGGCTGGGAGGCCTCCTCCGAGCGGATGTACCCC |
| | GAGGACGGCGCCCTGAAGGGCGAGATCAAGCAGAGGCT |
| | GAAGCTGAAGGACGGCGGCCACTACGACGCTGAGGTCAA |
| | GACCACCTACAAGGCCAAGAAGCCCGTGCAGCTGCCCGG |
| | CGCCTACAACGTCAACATCAAGTTGGACATCACCTCCCAC |
| | AACGAGGACTACACCATCGTGGAACAGTACGAACGCGCC |
| | GAGGGCCGCCACTCCACCGGCGGCATGGACGAGCTGTAC |
| | AAGTAA (SEQ ID NO: 24) |

### Chimeric PGCs and Chimeric Avians

As used herein, the term “chimeric PGC” refers to a gene-edited or genetically modified avian PGC that contains the DNA editing agent disclosed herein, either a gene-edited or genetically modified PGC transformed by genetic modification or a PGC from a gene-edited or genetically modified avian (chimeric avian). In some embodiments, provided herein is a cell colony comprising gene-edited or genetically modified avian PGCs derived from a single individual parent PGC. In some embodiments, provided herein is a cell population comprising gene-edited or genetically modified avian cells, the cell population derived from a single parent PGC.

As used herein, the term “chimera”, “chimeric chick,” “chimeric adult avian,” or “chimeric avian” refers to a bird cell that contains the DNA editing agent disclosed herein, or a bird that has cells containing the DNA editing agent disclosed herein. Representative examples of chimeric bird cells include, but are not limited to, bird primordial germ cells (PGCs) such as gonadal PGCs, blood PGCs, germinal crescent PGCs, or gametes that contain the DNA editing agent disclosed herein. Representative examples of chimeric bird include, but are not limited to, chicken, turkey, duck, geese, quail, pheasant, or ostrich that has cells containing the DNA editing agent disclosed herein.

In certain embodiments, the cells of the bird comprising the exogenous polynucleotide cassette comprise bird primordial germ cells (PGCs). In certain embodiments, the bird PGCs can be gonadal PGCs, blood PGCs, or germinal crescent PGCs.

In certain embodiments, the cells of the bird comprising the exogenous polynucleotide cassette comprise bird primordial germ cells (PGCs). In certain embodiments, the bird PGCs can be gonadal PGCs, blood PGCs, or germinal crescent PGCs.

As used herein, the terms “primordial germ cell” and “PGC” refer to a diploid cell that is present in the early embryo and that can differentiate/develop into haploid gametes (i.e. spermatozoa and ova) in an adult bird.

In certain aspects, provided herein are methods for producing a sterile gene-edited or genetically modified avian, the method comprising: (a) obtaining a PGC from an avian; (b) stably integrating into a GOI on a chromosome of interest in the PGC a first exogenous polynucleotide which is operatively linked to a recombinase recognition site, the first exogenous polynucleotide eliciting a sterility-inducing phenotype in the PGC or in a gene-edited or genetically modified avian derived from the PGC, and stably integrating into the targeted GOI on the chromosome of interest in the PGC a second exogenous polynucleotide encoding a recombinase enzyme, (i) wherein the first exogenous polynucleotide encodes a sterility-inducing protein or an endonuclease enzyme that can carry out genome editing; or (ii) wherein insertion of the first exogenous polynucleotide in the chromosome of interest modifies or disrupts the GOI, the GOI having: (1) an isolated function specific to a PGCC; or (2) a function specific to gametogenesis, gamete maturation, or gamete function in the gene-edited or genetically modified avian; (c) producing pure PGC colonies, the PGC colonies comprising the first exogenous polynucleotide and the second exogenous polynucleotide; (d) transplanting a pure PGC colony to a male chick embryo to produce a chimera male chick embryo and transplanting a pure PGC colony to a female chick embryo to produce a chimera female chick embryo; (e) hatching and rearing the chimera founder chick to sexual maturity as a chimera founder adult avian; (f) screening the chimera founder adult avian to verify heterozygosity for the edited GOI; (g) breeding a male chimera founder adult avian having heterozygosity for the edited GOI with a female chimera founder adult avian having heterozygosity for the edited GOI to produce progeny embryos; and (h) identifying a sterile homozygotic embryo from the progeny embryos. Also provided herein are methods further comprising: (i) providing a desired PGC having a desired trait of interest; and (j) transplanting the desired PGC into the sterile homozygotic embryo.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As is known to those of skill in the art, primordial germ cells can be isolated from different developmental stages and from various sites in a developing avian embryo such as, but not limited to, genital ridge, developing gonad, blood, and germinal crescent (Chang et al., Cell Biol Int 21:495-9, 1997; Chang et al., Cell Biol Int 19:143-9, 1995; Allioli et al., Dev Biol 165:30-7, 1994; Swift, Am J Physiol 15:483-516; PCT International Publication No. WO 99/06533). The genital ridge is a section of a developing embryo that is known to a person of ordinary skill in the art (Strelchenko, Theriogenology 45: 130-141, 1996; Lavoir, J Reprod Dev 37: 413-424, 1994). Typically, PGCs can be stained positively by the periodic acid-Schiff (PAS) technique. In several species, PGCs can be identified using an anti-SSEA antibody (one notable exception being turkeys, the PGCs from which do not display the SSEA antigen). Various techniques for isolation and purification of PGCs are known in the art, including the concentration of PGCs from blood using Ficoll density gradient centrifugation (Yasuda et al., J Reprod Fertil 96:521-528, 1992).

The in-vitro culture of PGCs is possible using a medium containing chicken and bovine serum, conditioned media, feeder cells and growth factors such as FGF2 (van de Lavoir et al. 2006, Nature 441:766-769. doi:10.1038/nature04831; Choi et al. 2010, PLoS ONE 5:e12968. doi:10.1371/journal.pone.0012968; MacDonald et al., 2010. PLoS ONE 5:e15518. doi:10.1371/journal.pone.0015518). It has been shown that a feeder replacement medium containing growth factors to activate the FGF, insulin and TGF-β signaling pathways could be used to propagate PGCs (Whyte et al. 2015, Stem Cell Rep 5:1171-1182. doi:10.1016/j.stemcr.2015.10.008).

Primordial germ cells (PGCs) can be provided and formulated for carrying out the presently disclosed subject matter by any suitable technique, and stored, frozen, cultured, or the like prior to use as desired. For example, primordial germ cells can be collected from donor embryos at an appropriate embryonic stage. Stages of avian development are referred to herein by one of two art-recognized staging systems: the Eyal-Giladi & Kochav system (EG&K; Eyal-Giladi & Kochav, Dev Biol 49:321-327, 1976), which uses Roman numerals to refer the pre-primitive streak stages of development, and the Hamburger & Hamilton staging system (H&H; Hamburger & Hamilton, J Morphol 88:49-92, 1951), which uses Arabic numerals to reference the post-laying stages. Unless otherwise indicated, the stages referred to herein are stages as per the H&H staging system. In certain embodiments, PGCs are derived from blood isolated from stage 14 (H&H) embryos. In certain embodiments, PGCs are derived from blood isolated from stage 15 (H&H) embryos. In certain embodiments, PGCs are derived from blood isolated from stage 16 (H&H) embryos.

In one embodiment, PGCs can be isolated at stage 4, or the germinal crescent stage, through stage 30, with cells being collected from blood, genital ridge, or gonad in the later stages. The primordial germ cells are, in general, twice the size of somatic cells and can easily be distinguished and separated on the basis of size. Male (or homogametic) primordial germ cells (ZZ) can be distinguished from heterogametic primordial germ cells (ZW by any suitable technique, such as collecting germ cells from a particular donor and typing other cells from that donor, the collected cells being of the same chromosome type as the typed cells.

An alternative to the use of PGCs is the direct transfection of spermatozoa using a DNA editing agent disclosed herein (Cooper et al., 2016 Transgenic Res 26:331-347, doi:10.1007/s11248-016-0003-0).

In one embodiment, to produce chimeric birds from PGCs edited in-vitro, the exogenous edited cells are injected intravenously into surrogate host embryos at a stage when their endogenous PGCs are migrating to the genital ridge. The “donor” PGCs may be of the same breed or species as the surrogate host embryo or of a different breed or species. The edited “donor” PGCs must remain viable and in one embodiment, out-compete the endogenous PGCs if they are to colonize the forming gonad and transmit the edited chromosome(s) through the germline. To provide donor PGCs with an advantage, the number of endogenous PGCs can be reduced by chemical or genetic ablation (Smith et al., 2015, Andrology 3:1035-1049. doi:10.1111/andr.12107). Exposing the blastoderm of surrogate embryos to emulsified Busulfan has been shown to increase germline transmission of donor PGCs to over 90%, though this rate drops significantly if PGCs have been cultured or cryopreserved (Nakamura et al., 2008, Reprod Fertil Dev 20:900-907. doi:10.1071/RD08138; Naito et al., 2015, Anim Reprod Sci. 153:50-61. doi:10.1016/j.anireprosci.2014.12.003). Other methods of skewing the ratio of edited PGCs to native PGCs are described in US Application No. 2006/0095980.

In certain embodiments, genetically modified PGCs can be transplanted into adult gonads as known in the art (Trefil et al., 2017 Sci Rep, October 27; 7(1):14246 doi: 10.1038/541598-017-14475-w).

The genetically modified cells (e.g. PGCs) can be formulated for administration to other birds by dissociating the cells (e.g., by mechanical dissociation) and intimately admixing the cells with a pharmaceutically acceptable carrier (e.g., phosphate buffered saline solution). In one embodiment, the primordial germ cells are gonadal primordial germ cells, or blood primordial germ cells (“gonad” or “blood” referring to the tissue of origin of the original embryonic donor). In one embodiment, the PGCs can be administered in physiologically acceptable carrier at a pH of from about 6 to about 8 or 8.5, in a suitable amount to achieve the desired effect (e.g., 100 to 30,000 PGCs per embryo). The PGCs can be administered free of other ingredients or cells, or other cells and ingredients can be administered along with the PGCs.

Administration of the primordial germ cells to the recipient animal in-ovo can be carried out at any suitable time at which the PGCs can still migrate to the developing gonads. In one embodiment, the administration is carried out from about stage IX according to the Eyal-Giladi & Kochav (EG&K) staging system to about stage 30 according to the Hamburger & Hamilton staging system of embryonic development, or in another embodiment, at stage 15. For chickens, the time of administration is thus during days 1, 2, 3, or 4 of embryonic development, for example, day 2 to day 2.5. Administration is typically done by injection into any suitable target site, such as the region defined by the amnion (including the embryo), the yolk sac, etc. In one embodiment, the cells are injected into the embryo itself (including the embryo body wall). In alternative embodiments, intravascular or intracoelomic injection into the embryo can be employed. In other embodiments, the injection is performed into the heart. The methods of the presently disclosed subject matter can be carried out with prior sterilization of the recipient bird in-ovo (e.g. by chemical treatment using Busulfan of by gamma or X-ray irradiation). As used herein, the term “sterilization” refers to render partially or completely incapable of producing gametes derived from endogenous PGCs. When donor gametes are collected from such a recipient, they can be collected as a mixture with gametes of the donor and the recipient. This mixture can be used directly, or the mixture can be further processed to enrich the proportion of donor gametes therein. Thus, a skilled artisan would appreciate that a sterile avian disclosed herein comprises an avian with a reduced capacity for producing gametes derived from endogenous PGCs, compared to an isogenic avian lacking the first genetic modification.

The in-ovo administration of the primordial germ cells can be carried out by any suitable technique, either manually or in an automated manner. In one embodiment, in-ovo administration is performed by injection. The mechanism of in-ovo administration is not critical, but the mechanism should not unduly damage the tissues and organs of the embryo or the extraembryonic membranes surrounding it so that the treatment will not unduly decrease hatch rate. A hypodermic syringe fitted with a needle of about 18 to 26 gauge is suitable for the purpose. A sharpened pulled glass pipette with an opening of about 20-50 microns diameter may be used. Depending on the precise stage of development and position of the embryo, a one-inch needle will terminate either in the fluid above the chick or in the chick itself. A pilot hole can be punched or drilled through the shell prior to insertion of the needle to prevent damaging or dulling of the needle. If desired, the egg can be sealed with a substantially bacteria-impermeable sealing material such as wax or the like to prevent subsequent entry of undesirable bacteria. It is envisioned that a high-speed injection system for avian embryos would be suitable for practicing the presently disclosed subject matter. All such devices, as adapted for practicing the methods disclosed herein, comprise an injector containing a formulation of the primordial germ cells as described herein, with the injector positioned to inject an egg carried by the apparatus. In addition, a sealing apparatus operatively connected to the injection apparatus can be provided for sealing the hole in the egg after injection. In another embodiment, a pulled glass micropipette can be used to introduce the PGCs into the appropriate location within the egg, for example directly into the blood stream, either to a vein or an artery or directly into the heart.

Once the eggs have been injected with the modified PGCs, the chimeric embryo is incubated until hatch. In one embodiment, the chick is raised to sexual maturity, wherein the chimeric bird produces gametes derived from the donor PGCs.

In certain embodiments, the cells of the bird comprise bird gametes. The gametes, (either eggs or sperm) from the chimeras (or from material that has been directly genetically manipulated, as described herein above) are then used to raise founder chickens (F1). Molecular biology techniques known in the art (e.g. PCR or Southern blot or both) may be used to confirm germline transmission. F1 chickens may be back-crossed to generate homozygous carrier males and carrier females (F2). Gametes from founder chickens F2 can then be used to expand the breeding colonies. The colonies are typically grown until sexual maturity.

In certain embodiments, the method further comprises incubating the chimeric bird embryo, in-ovo, until hatching. In certain embodiments, the method further comprises raising the chimeric bird to sexual maturity, wherein the chimeric bird produces gametes derived from the administered cells.

In certain embodiments, the genome-edited cells are administered by in-ovo injection. In certain embodiments, the administrated cell population is derived from the same avian species as the recipient bird embryo. In certain embodiments, the administrated cell population is derived from a different avian species as the recipient bird embryo.

In certain embodiments, the genome-edited bird cell population is administered when the recipient embryo is about stage IX according to the Eyal-Giladi & Kochav staging system. In certain embodiments, the bird cell population is administered when the recipient embryo is about stage 30 according to the Hamburger & Hamilton staging system. In certain embodiments, the bird cell population is administered when the recipient embryo is about stage IX according to the Eyal-Giladi & Kochav staging system; and about stage 30 according to the Hamburger & Hamilton staging system. In certain embodiments, the bird cell population is administered when the recipient embryo is after stage 14 according to the Hamburger & Hamilton staging system.

In certain embodiments, the genome-edited bird cell population is administered after irradiation of the embryo. In certain embodiments, the irradiation comprises γ-irradiation or X-ray irradiation. In certain embodiments, the irradiation comprises 600-800 rad of γ irradiation. In certain embodiments, the irradiation comprises 600-800 rad of irradiation. In certain embodiments, the irradiation comprises 400-1000 rad of irradiation. In certain embodiments, the irradiation comprises 200-1200 rad of irradiation.

Further provided, in another aspect, is a chimeric bird obtainable from the methods described above.

### Genome Editing and Transforming PGCs in Chickens

Transcription Activator-like Effector Nuclease (TALEN), Zinc finger nucleases, and Clustered Regularly Interspaced Short Palindromic Repeats (CRISPR)-Cas9 (Taylor et al. [2017] Devel. (Cambridge, England) 144: 928-934) are among the variety of useful tools for genome editing in chickens. For example, CRISPR-Cas9 benefits from easy construction, rapid application and high efficiency.

In some embodiments, the CRISPR-Cas9 system is introduced to PGCs as transient episomal plasmids or as a recombinant sgRNA-Cas9 protein complex (RNP). Apart from the genome editing per se, the strategies leave no alien DNA traces of the CRISPR system. Generating DNA breaks invokes the DNA repair mechanism that could result in INDELs (i.e., insertion or deletion of 1-10,000 bases in the genome of the organism) in the break site. These INDELs may render the targeted gene inactive. This inactivation can be achieved by the non-homologous-end-joining repair mechanism. On the other hand, the homologous recombination repair mechanism facilitates targeted integration of a foreign DNA sequence, which can replace the genomic DNA sequence flanking the break point. Using targeted integration to the genome, a gene of interest (GOI), such as a reporter gene, could substitute for an endogenous gene to be under the regulation of the endogenous promoter. To this end, PGCs are co-transfected with the CRISPR system expressing plasmid with a targeting vector (TV) plasmid which contains a desired genomic modification to be introduced. With relevance to some embodiments, the coding sequence of the reporter gene mCherry is introduced and replaces the coding sequence of DAZL which is expressed in PGCs (see FIG. 1B). This results in the expression of mCherry in all PGCs, such that they fluoresce in a red color to indicate transformation.

In other embodiments, to knockout the expression of a sterility-inducing gene, a conditional-activated mechanism can also be used, such as the Cre-LoxP or Flp-FRT systems. To this end, using homologous recombination, two LoxP sites (for example) can be introduced in intronic regions, flanking an essential exon. This method allows for normal expression of the gene. However, by crossing with Cre expressing strain, the LoxP flanked exon is removed, and the gene becomes inactive.

Importantly, the rate of intrinsic homologous recombination events in PGCs, without DNA break, is high. Thus, transfection of targeting vector alone can result in a specific homologous recombination-mediated genomic integration, without risking damaging the genomic DNA by off-target modifications known to be generated by genome editing agents.

### Gene Knockout

In some embodiments, the gene-editing method for producing sterile chicks comprises a gene knockout method. In some embodiments, the genome editing techniques above (e.g., specific gene knockout chickens via TALEN-mediated, CRISPR/Cas9, or other methods known in the art) are adapted to be used to knock out a gene (e.g., to silence its expression), including, but not limited to removal, replacement, inactivation, mutation, and the like for knocking out or otherwise silencing or inactivating the gene. Other methods include, but are not limited to, the use of viral vectors (e.g., avian leukosis virus, lentivirus, retrovirus, and other vectors), e.g., via microinjection, electroporation, or other techniques.

### Genetically Modified Chicks

In some embodiments, the method for producing sterile chicks comprises genetic modification to alter the genome of the chick. In some embodiments, the altered genome comprises a DNA sequence or gene from a different species to produce a transgenic chick.

Genetic modification methods include, but are not limited to, the methods described herein, including the use of endonucleases and genome modifiers, such as CRISPR, TALEN, etc., with or without the combination of homologous recombination repair mechanisms. Examples of DNA sequences or genes from a different species include, but are not limited to, the examples described herein, including green fluorescent protein (e.g., jellyfish Aequorea Victoria), 3-phosphoglycerate kinase (Pgk) promoter (e.g., mouse), cytomegalovirus (CMV) promoter (cytomegalovirus), internal ribosome entry site (IRES) (poliovirus [PV]), and encephalomyocarditis virus (EMCV).

Unless otherwise indicated, all numbers expressing quantities, ratios, and numerical properties of ingredients, reaction conditions, and so forth used in the specification and claims are to be understood as being modified in all instances by the term “about”. All parts, percentages, ratios, etc. herein are by weight unless indicated otherwise.

As used herein, the singular forms “a” or “an” or “the” are used interchangeably and intended to include the plural forms as well and fall within each meaning, unless expressly stated otherwise or unless the context clearly dictates otherwise. For example, the term “a compound” or “at least one compound” may include a plurality of compounds, including mixtures thereof.

Also as used herein, “at least one” is intended to mean “one or more” of the listed elements. Singular word forms are intended to include plural word forms and are likewise used herein interchangeably where appropriate and fall within each meaning, unless expressly stated otherwise. Except where noted otherwise, capitalized and non-capitalized forms of all terms fall within each meaning.

“Consisting of” shall thus mean excluding more than traces of other elements. The skilled artisan would appreciate that while, in some embodiments the term “comprising” is used, such a term may be replaced by the term “consisting of”, wherein such a replacement would narrow the scope of inclusion of elements not specifically recited. The terms “comprises”, “comprising”, “includes”, “including”, “having” and their conjugates encompass “including but not limited to”.

The term “about” or “approximately” means within an acceptable error range for the particular value as determined by one of ordinary skill in the art, which will depend in part on how the value is measured or determined. In some embodiments, the term “about” refers to a deviance of between 0.0001-5% from the indicated number or range of numbers. In some embodiments, the term “about” refers to a deviance of between 1-10% from the indicated number or range of numbers. In some embodiments, the term “about” refers to a deviance of up to 25% from the indicated number or range of numbers. In some embodiments, the term “about” refers to ±10%.

Throughout this application, various embodiments may be presented in a range format. It should be understood that the description in range format is merely for convenience and brevity and should not be construed as an inflexible limitation on the scope of certain embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Any patent, patent application publication, or scientific publication, cited herein, is incorporated by reference herein in its entirety.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Production of Sterile Avian Embryos Devoid of Functional Primordial Germ Cells (PGCs) Using Pooled PGCs

Objective: To produce sterile avian embryos devoid of functional PGCs.

PGCs of a desired avian species or breed are isolated and cultured. A gene of interest (GOI), the product of which has a function related to fertility (e.g., having an isolated function in PGCs, gamete maturation, or gamete function), is identified, such that deletion or mutation of the gene product will lead to sterility, preferably without having an impact on viability. The PGCs are subjected to targeted genome editing of the GOI and cultured.

Pooled PGC cells are transplanted to an avian embryo, thereby creating a chimera avian embryo having both mutant (various mutations) and naturally occurring PGCs. The chimera offspring are hatched and reared to sexual maturity, then screened for heterozygosity for the edited GOI in order to identify founders.

Male and female heterozygote founder pairs are bred, and the sterile embryos are identified and retrieved.

### Example 2: Production of Sterile Avian Embryos Devoid of Functional Primordial Germ Cells (PGCs) Using Pure or Enriched PGCs

Objective: To produce sterile avian embryos devoid of functional PGCs.

PGCs of a desired avian species or breed are isolated and cultured. A gene of interest (GOI), the product of which has a function related to fertility (e.g., having an isolated function in PGCs, gamete maturation, or gamete function), is identified, such that deletion or mutation of the gene product will lead to sterility, preferably without having an impact on viability. The PGCs are subjected to targeted genome editing of the GOI and cultured. Pure or enriched PGC colonies are obtained (e.g., via fluorescence-activated cell sorting [FACS]), and the edited genome is analyzed or optionally verified as containing the mutation or ablation of the GOI.

Selected pure PGC colonies having verified mutation/ablation of the GOI (or selected enriched PGC colonies) are transplanted to an avian embryo, thereby creating a chimera avian embryo having both mutant and naturally occurring PGCs. The chimera offspring are hatched and reared to sexual maturity, then screened for heterozygosity for the edited GOI in order to identify founders.

Male and female heterozygote founder pairs are bred, and the sterile embryos are identified and retrieved. (Theoretically, where pure PGC colonies are used, homozygote sterile embryos should occur at a 25% Mendelian ratio, provided that the mutation or ablation does not have an impact on viability.)

### Example 3: Production of Sterile Avian Embryos that Will Lay Eggs of Birds Having Desirable Traits

Objective: To produce sterile avian embryos that will lay eggs of birds having desirable traits.

Sterile avian embryos are produced according to the methods described herein (e.g., Example 1 or Example 2).

Donor PGCs having a desirable trait are transplanted into the surrogate/host sterile avian embryos so that the sterile avian embryos will lay eggs of birds having desirable traits.

### Example 4: Production of Female Birds Having Good Reproduction Capabilities that Will Lay Eggs of Birds Having Other Desirable Traits

Objective: To produce female birds having good reproduction capabilities that will lay eggs of birds having another desired trait.

This example exploits the benefits of both breeds by generating a female bird having exceptional reproduction capabilities, which will lay eggs from which young birds having another desired trait will hatch. Of particular significance, these hatchlings will be otherwise identical to hatchings currently used by the industry, namely non-GMO.

PGCs from highly performing birds having a desired trait are cultured and transplanted into sterile, reproductively strong embryos, as described above. These host embryos will hatch, and upon reaching sexual maturity, by standard interface breeding, will give rise to hatchlings having the desired trait. Since PGCs can be practically expended in culture for many generations, it is possible to select for the highest preforming birds having the desired trait and to use their PGCs as donors, thereby exploiting even further their desired properties.

### Example 5: Production of Female Birds that Will Lay Genetically Transformed Eggs

Objective: To produce female birds that will lay genetically transformed eggs.

This example exploits the benefits of multiples types of birds by generating a female with exceptional reproduction capabilities, which will lay eggs from which genetically modified young birds will hatch.

PGCs are genetically transformed. One or more modifications are made that improve, e.g., agricultural performances, health, disease resistance, resilience to various stress conditions, behavior characteristics, and can also be used to introduce traits which are not naturally exist in birds of a particular species.

Genetically transformed PGCs are injected into sterile embryos that will hatch, and upon reaching to breeding stage, will produce genetically modified hatchlings that originate from the genetically transformed PGCs, using the methods as described herein.

### Example 6: Production of Sterile Birds for Cryopreservation

Objective: To produce birds for cryopreservation or cryo-banking purposes.

PGCs are collected from one of the various embryonic stages from the freshly laid egg, the germinal crescent, the blood stream, or directly from gonads. When sufficient amount of PGCs are obtained, either by direct collection, e.g., from the gonads, or following culturing, PGCs can be cryopreserved in liquid nitrogen, for many years, and once thawed they can be injected as donor PGCs to a sterile host embryo, using the methods as described herein. Thus, generating sterile embryos will allow an easy and reliable retrieval of cryopreserved colonies, e.g., for industrial breeds as well for non-industrial breeds, for both females and males. Of particular significance, while the sterile embryo genome is modified, the end-product in this application is not. The retrieved breeds are genetically identical to the wild-type source of the PGCs.

### Example 7: Production of Sterile Birds for Developing Genome-Edited or Modified Breeds or Both

Objective: To produce sterile birds for developing genome-edited or modified breeds or both.

Current methods for generating genome-edited avian breeds entail a multi-step process. Following genomic transformation, the genome-modified PGCs are currently injected to a surrogate recipient host embryo alongside to its endogenous PGCs, thereby giving rise to a “chimera,” and the two populations of PGCs colonize the gonad. The ratio between the endogenous and modified PGCs in the gonads, and their potential to give rise to functional gametes, is reflected in the germline transmission (Macdonald et al. 2012), which is variable. Low germline transmission ratio results in months of laborious screening for founder chicks which originate from modified PGCs.

However, injecting modified PGCs into a sterile embryo (e.g., of the same species), using the methods as described herein, will result in 100% transmission rate as all the gametes will originate from the injected PGCs, effectively making screening redundant, thus saving time and efforts in creating future new genome-edited avian breeds. Moreover, by generating surrogate sterile chickens, from both sexes, harboring PGCs with similar genetic modification, it is possible to breed the two and obtain homozygotes, already on the first generation, thus again saving time and efforts.

### Example 8: Production of Sterile Female Birds that Will Lay Eggs Having Genetically Modified Characteristics

Objective: To produce sterile birds that will lay eggs having genetically modified characteristics.

The ability to genetically modify PGCs, and to have these PGCs mature to gametes in sterile female birds opens an opportunity to solve numerous issues in the poultry and game industries. For example, solving the need to cull day-old males due to a breeding or producing preference for females. Within these industries, birds of a non-desired gender are an inevitable by-product of the industry, and thus, they are manually sorted and culled. Sex determination in young birds is based on combinatorial segregation of the sex chromosomes Z and W in the female's gametes. All male birds harbor a Z chromosome which segregates from their respective mothers. Introducing an embryonic lethality-inducing gene on this chromosome will render all male embryos to cease developing soon after fertilization. Thus, only female birds will survive embryogenesis, will hatch normally, and will mature to egg-laying adult females.

### Example 9: Production of Sterile Chicken Embryos Devoid of Functional Primordial Germ Cells (PGCs) Using Pooled PGCs

Objective: To produce sterile chicken embryos devoid of functional PGCs.

PGCs of a desired chicken breed are isolated and cultured. A gene of interest (GOI), the product of which has a function related to fertility (e.g., having an isolated function in PGCs, gamete maturation, or gamete function), is identified, such that deletion or mutation of the gene product will lead to sterility. The PGCs are subjected to targeted genome editing of the GOI and cultured.

Pooled PGC cells are transplanted to an avian embryo, thereby creating a chimera avian embryo having both mutant (various mutations) and naturally occurring PGCs. The chimera offspring are hatched and reared to sexual maturity, then screened for heterozygosity for the edited GOI in order to identify founders.

Male and female heterozygote founder chick pairs are bred, and the sterile embryos are identified and retrieved.

### Example 10: Production of Sterile Chicken Embryos Devoid of Functional Primordial Germ Cells (PGCs) Using Pure or Enriched PGCs

Objective: To produce sterile chicken embryos devoid of functional PGCs.

PGCs of a desired chicken breed are isolated and cultured. A gene of interest (GOI), the product of which has a function related to fertility (e.g., having an isolated function in PGCs, gamete maturation, or gamete function), is identified, such that deletion or mutation of the gene product will lead to sterility. The PGCs are subjected to targeted genome editing of the GOI and cultured. Pure PGC (or enriched) colonies are obtained (e.g., via FACS), and the edited genome is analyzed or optionally verified as containing the mutation or ablation of the GOI.

Selected pure PGC colonies having verified mutation/ablation of the GOI (or selected enriched PGC colonies) are transplanted to a chick embryo, thereby creating a chimera chick embryo having both mutant and naturally occurring PGCs. The chimera chicks are hatched and reared to sexual maturity, then screened for heterozygosity for the edited GOI in order to identify founder chicks.

Male and female heterozygote founder chick pairs are bred, and the sterile embryos are identified and retrieved. (Theoretically, where pure PGC colonies are used, homozygote sterile embryos should occur at a 25% Mendelian ratio, provided that the mutation or ablation does not have an impact on viability.)

### Example 11: Production of Sterile Chick Embryos that Will Lay Eggs of Birds Having Desirable Traits

Objective: To produce sterile chick embryos that will lay eggs of chickens having desirable traits.

Sterile avian embryos are produced according to the methods described herein (e.g., Example 1, Example 2, or any of Examples 8-10).

Donor chicken PGCs having a desirable trait are transplanted into the surrogate/host sterile chick embryos so that the sterile chick embryos will lay eggs of chickens having desirable traits.

### Example 12: Production of Layer-Type Hens that Will Lay Broiler-Type Eggs

Objective: To produce layer-type hens that will lay broiler-type eggs.

This example exploits the benefits of both breeds by generating a layer-type hen, with exceptional reproduction capabilities, which will lay eggs from which broiler chicks will hatch. Of particular significance, these broilers will be otherwise identical to broilers chicks currently used by the industry, namely non-GMO.

PGCs from highly preforming broilers are cultured and transplanted into sterile, layer-type embryos, as described above. These host embryos will hatch, and upon reaching sexual maturity, by standard interface breeding, will give rise to broilers. Since PGCs can be practically expended in culture for many generations, it is possible to select for the highest preforming broilers and to use their PGCs as donors, thereby exploiting even further their high growth rate and low feed conversion ratio (FCR) properties. This strategy also benefits from increasing uniformity in broiler flocks and production workflow of broiler meat.

### Example 13: Production of Layer-Type Hens that Will Lay Genetically Transformed Eggs

Objective: To produce layer-type hens that will lay genetically transformed eggs.

This example further exploits the benefits of both breeds by generating a layer-type hen, with exceptional reproduction capabilities, which will lay eggs from which genetically modified chicks will hatch.

PGCs are genetically transformed. Modifications are made that improve, e.g., agricultural performances, health, disease resistance, resilience to various stress conditions, behavior characteristics, and can also be used to introduce traits which are not naturally exist in chickens.

Genetically transformed PGCs are injected into sterile embryos that will hatch, and upon reaching to breeding stage, will produce chicks that originate from the genetically transformed PGCs, as described above.

### Example 14: Production of Sterile Female Chickens for Cryopreservation

Objective: To produce sterile chickens for cryopreservation or cryo-banking purposes.

PGCs is collected from one of the various embryonic stages, e.g., from the freshly laid egg, the germinal crescent, the blood stream, or directly from gonads. When sufficient amounts of PGCs are obtained, either by direct collection, e.g., from the gonads, or following culturing, PGCs can be cryopreserved in liquid nitrogen for many years, and once thawed they can be injected as a donor PGCs to a sterile host embryo, using the methods as described herein. Thus, generating sterile chicken embryos will allow an easy and reliable retrieval of cryopreserved colonies for both broiler and layer industry breeds, as well for non-industrial breeds, for both females and males. Of particular significance, while the sterile embryo genome is modified, the end-product in this application is not. The retrieved breeds are genetically identical to the WT source of PGCs.

### Example 15: Production of Sterile Chickens for Developing Genome-Edited or Modified Breeds or Both

Objective: To produce sterile chickens for developing genome-edited or modified breeds or both.

Current methods for generating genome-edited avian breeds entail a multi-step process. Following genomic transformation, the genome-modified PGCs are currently injected to a surrogate recipient host embryo alongside to its endogenous PGCs, thereby giving rise to a “chimera,” and the two populations of PGCs colonize the gonad. The ratio between the endogenous and modified PGCs in the gonads, and their potential to give rise to functional gametes, is reflected in the germline transmission (Macdonald et al. 2012), which is variable. Low germline transmission ratio results in months of laborious screening for founder chicks which originate from modified PGCs.

However, injecting modified PGCs into a sterile embryo, using the methods as described herein, will result in 100% transmission rate as all the gametes will originate from the injected PGCs, effectively making screening redundant, thus saving time and efforts in the creating future new genome-edited chicken breeds. In some examples, the origin breed of the donor chicken and the origin breed of the host chicken are identical. In other examples, the origin breed of the donor chicken and the origin breed of the host chicken are different (e.g., a donor broiler chicken and a host layer-type chicken).

### Example 16: Production of Sterile Female Chickens that Will Lay Eggs Having Genetically Modified Characteristics

Objective: To produce sterile birds that will lay eggs having genetically modified characteristics.

The ability to genetically modified PGCs, and to have these PGCs mature to gametes in sterile chickens opens an opportunity to solve numerous issues in the poultry and game industries. For example, solving the need to cull day-old male chicks due to a breeding or producing preference for female chicks in the layer-type chicken industry. Within this industry, male chicks are an inevitable by-product of the industry, and thus, they are manually sorted and culled, a labor-intensive process, particularly in view of the similarity of appearance between male and female chicks. Sex determination in chickens is based on combinatorial segregation of the sex chromosomes Z and Win the hen's gametes. All male chicks harbor a Z chromosome which segregates from their respective mothers. Introducing an embryonic lethality-inducing gene on this chromosome will render all male embryos to cease developing soon after fertilization. Thus, only female chicks will survive embryogenesis, will hatch normally, and will mature to table-egg laying hens.

### Example 17: Production of Sterile Embryos Devoid of Functional Primordial Germ Cells (PGCs)

Objective: To produce sterile chicken embryos devoid of functional PGCs.

Methods: Generating a genome-edited chicken for the production of eggs containing sterile embryos is a multi-step process which begins with isolation and culturing of PGCs; targeted genome-editing of a gene of interest (GOI) in the PGCs and obtaining colonies of pure edited PGCs; verification of the edited genome and selection of pure PGC colonies having the edited GOI; transplantation of pure PGCs colonies comprising the edited GOI to chimera embryos; incubating and hatching the chimeric chicks and then rearing the chimera chicks to sexual maturity; screening the chimeric chicks for founder heterozygous chicks with germline transmission in both their somatic cells and in their gametes, which therefore includes the edited GOI; breeding the heterozygote founder chicks to produce mature chickens; and collecting the sterile eggs (embryos) for further use. Embodiments of certain methods may be found, for example but not limited to International Application Publication No. WO 2019/058376, which is incorporated in its entirety, in particular, the Example Section.

Alternative methods: Additionally, generating a genome-edited chicken for the production of eggs containing sterile embryos is a multi-step process which begins with isolation and culturing of PGCs; targeted genome-editing of a gene of interest (GOI) in the PGCs and obtaining colonies of enriched PGCs; analysis or optional verification of the edited genome and optional selection of pure or enriched PGC colonies having the edited GOI; transplantation of PGCs colonies to chimera embryos; incubating and hatching the chimeric chicks and then rearing the chimera chicks to sexual maturity; screening the chimeric chicks for founder heterozygous chicks with germline transmission in both their somatic cells and in their gametes, which therefore includes the edited GOI; breeding the heterozygote founder chicks to produce mature chickens; and collecting the sterile eggs (embryos) for further use. Further still, generating a genome-edited chicken for the production of eggs containing sterile embryos is a multi-step process which begins with isolation and culturing of PGCs; targeted genome-editing of a gene of interest (GOI) in the PGCs and obtaining pools of PGCs; transplantation of pooled PGCs colonies to chimera embryos; incubating and hatching the chimeric chicks and then rearing the chimera chicks to sexual maturity; screening the chimeric chicks for founder heterozygous chicks with germline transmission in both their somatic cells and in their gametes, which therefore includes the edited GOI; breeding the heterozygote founder chicks to produce mature chickens; and collecting the sterile eggs (embryos) for further use.

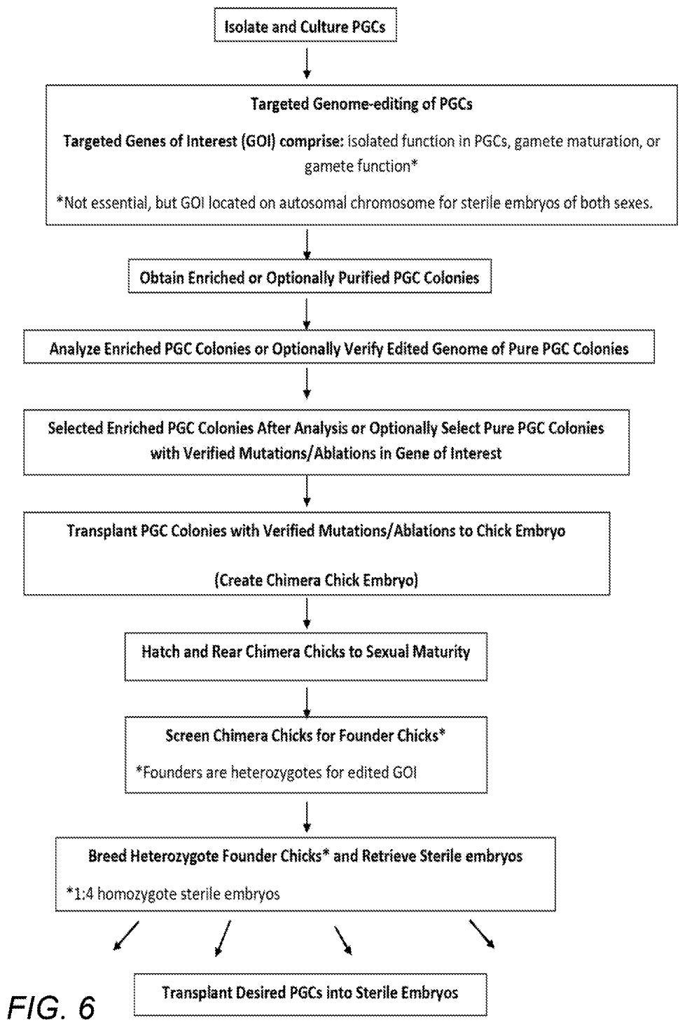

FIG. 6 provides an overview of the steps for producing sterile embryos devoid of functional PGCs. This non-limiting example, as provided in the FIGS., demonstrates knock-in of mCherry to the DAZL locus, thus knocking-out DAZL activity on this allele. However, any other method to knockout method sterility-inducing genes can similarly be used, without the need to introduce a reporter gene. Moreover, while genome editing tools clearly improve the efficiency of knocking-out/knocking-in genes, this method can also be achieved in a “genome editing-independent” way, e.g., using homologous recombination, without CRISPR, which works good in chicken PGCs and in many other cell types. Other methods for producing sterile embryos devoid of functional PGCs include, but are not limited to:

1. Inducing knockout in a sterility-inducing gene can be done by using “genome-editing agents,” such as CRISPRs, TALEN, Z-fingers, etc.

2. Replacing genomic sequence using homologous recombination (HR) can be done using various sizes of targeting vectors, from about 100 bp to introduce a STOP codon, a restriction enzyme site, etc. Larger targeting vectors can be used to remove larger DNA fragments (e.g., exons) or to knocking reporters or a combination thereof, as described herein (SEQ ID NO: 37).

3. HR by itself, without generating DNA double-stranded break (DSB) using “genome-editing agents” is less efficient but it benefits from better off-target effects.

4. Without a reporter gene, it is more difficult to identify cells which underwent heterozygous KO, on one allele. (Knocking both alleles may result in PGCs death). However, given reasonably good efficiency, and enough screening, these cells can be identified.

5. Sterility may also be induced by binding to, or modifying, the mRNA or by modifying the level or protein or by inhibiting the protein.

For example, inserting a STOP codon in the genomic DNA, at the beginning of the coding sequence (CDS) of DAZL will result in sterility. However, by generating a transgenic chicken which expresses Cas13 and gRNA which binds to the mRNA of DAZL, sterility will be induced as well, while the genomic DNA sequence of the GOI DAZL is kept unchanged.

As a result, blocking the activity of DAZL and causing sterility, can be induced by modifying the DNA, mRNA, and possibly at the protein levels.

Details of different embodiments of the steps are provided below.

Isolation and Culturing of Source PGCs

PGCs are embryonic germs cells that give rise to gametes in adults. In chicken embryos they are first evident in the blastoderm soon after the egg is laid. At this stage they consist of 20-100 cells. Thereafter, PGCs can be collected from embryos from the blastoderm, the Germinal Crescent, the blood, the Genital Ridge or the embryonic gonads. Throughout their migration pathway they preserve their totipotency characteristics.

PGCs were isolated at stage 4, or the germinal crescent stage, through stage 30, with cells being collected from blood, genital ridge, or gonad in the later stages. The primordial germ cells are, in general, twice the size of somatic cells and can easily be distinguished and separated on the basis of size. Male (or homogametic) primordial germ cells (ZZ) were distinguished from female (or heterogametic) primordial germ cells (ZW) by a variety of techniques, such as collecting germ cells from a particular donor and typing other cells from that donor, the collected cells being of the same chromosome type as the typed cells.

PGCs were collected from chicken embryos either by microdissection or by drawing blood. For example, PGCs were collected from blood by placing ˜1.0-3.0 μL of blood isolated from stage 14 to 16 (H&H) embryos in medium in a 48-well plate, wherein the medium was changed as needed; cells were allowed to grow and divide and then propagated at 2-4×10⁵ cells/ml medium. Cells were then frozen in PGC culture medium containing 10% DMSO, temperature was gradually decreased to −80° C., the frozen PGC are stored for 1-3 days, and transferred to liquid nitrogen.

In-vitro culture of PGCs was performed using a medium containing chicken and bovine serum, conditioned media, feeder cells and growth factors such as FGF2 (For non-limiting examples, See: van de Lavoir et al. 2006, Nature 441:766-769. doi:10.1038/nature04831; Choi et al. 2010, PLoS ONE 5:e12968. doi:10.1371/journal.pone.0012968; and MacDonald et al. 2010. PLoS ONE 5:e15518. doi:10.1371/journal.pone.0015518). Recently, it has been shown that a feeder replacement medium containing growth factors to activate the FGF, insulin and TGF-β signaling pathways could be used to propagate PGCs (Whyte et al. 2015, Stem Cell Rep 5:1171-1182. doi:10.1016/j.stemcr.2015.10.008). Furthermore, the use of ovotransferrin as a replacement for the iron-carrying proteins present in avian serum permitted feeder-free and serum-free propagation of PGCs, with cells maintaining a high rate of proliferation. Any of these media may be used in embodiments herein for culturing isolated PGCs in vitro.

Sex determination and PGC line characterization: Each PGC line was characterized for sexing, mRNA expression of PGC markers, and protein expression of the known PGCs marker SSEA1. DNA from the donor embryo was isolated and kept for future reference. For sexing, DNA from 2-4×10 5 PGCs cells was collected, re-suspended in tail buffer (102-T, VIAGEN™) containing 100 μg/ml Proteinase K (SIGMA™) and incubated at 55° C. for 3 hours. The Proteinase K was inactivated at 85° C. for 45 minutes. PCR for sex determination was performed with primers from W chromosome that target female chromosome (P17, P18) and Ribosomal S18 (P19, P20) as a control. For gene expression analysis, RNA was purified using TRIZOL™ reagent (SIGMA-ALDRICH™ or THERMO-FISHER SCIENTIFIC™) and 1 μg of RNA was used for cDNA library production by reverse transcription PCR reaction (GOSCRIPT™ Reverse transcriptase, PROMEGA™). The cDNA served as a template for PCR by using DAZL, Sox2, cPouV, Nanog, Klf4, cVH primers, P21-P22, P23-P24, P25-P26, P27-P28, P29-P30, P31-P32, respectively. The sequences for primers P17-P32 are shown in in Table 2 below.

TABLE 2
Primer Sequences.

| Primer | | | Sequence |
|---|---|---|---|
| Number | Primer Name | Direction | (SEQ ID NO:) |
| P17 | Fw W chromosome | FWD | CCCAAATATAACACGCTTCACT |
| | | | (SEQ ID NO: 38) |
| P18 | Rev W chromosome | REV | GAAATGAATTATTTTCTGGCGAC |
| | | | (SEQ ID NO: 39) |
| P19 | Fw S18 | FWD | AGCTCTTTCTCGATTCCGTG |
| | | | (SEQ ID NO: 40) |
| P20 | Rev S18 | REV | GGGTAGACACAAGCTGAGCC |
| | | | (SEQ ID NO: 41) |
| P21 | Fw DAZL | FWD | CAACTATCAGGCTCCACCAC |
| | | | (SEQ ID NO: 42) |
| P22 | Rev DAZL | REV | CTCAGACGGTTTTCAGGGTT |
| | | | (SEQ ID NO: 43) |
| P23 | Fw SOX2 | FWD | AGGCTATGGGATGATGCAAG |
| | | | (SEQ ID NO: 44) |
| P24 | Rev SOX2 | REV | GTAGGTAGGCGATCCGTTCA |
| | | | (SEQ ID NO: 45) |
| P25 | Fw cPouV | FWD | CGAGACCAACGTGAAGGGAA |
| | | | (SEQ ID NO: 46) |
| P26 | Rev cPouV | REV | CAGACCCGGACAACGTCTTT |
| | | | (SEQ ID NO: 47) |
| P27 | Fw NANOG | FWD | CTCTGGGGCTCACCTACAAG |
| | | | (SEQ ID NO: 48) |
| P28 | Rev NANOG | REV | AGCCCTGGTGAAATGTAGGG |
| | | | (SEQ ID NO: 49) |
| P29 | Fw KLF4 | FWD | AGCTCTCATCTCAAGGCACA |
| | | | (SEQ ID NO: 50) |
| P30 | Rev KLF4 | REV | GGAAAGATCCACTGCTTCCA |
| | | | (SEQ ID NO: 51) |
| P31 | Fw cvh | FWD | AGCACAGGTGGTGAACGAACCA |
| | | | (SEQ ID NO: 52) |
| P32 | Rev cvh | REV | TCCAGGCCTCTTGATGCTACCGA |
| | | | (SEQ ID NO: 53) |

Chimeras which have been injected with the same sex PGCs are retained. In addition, DNA from the donor embryo was isolated and kept for future reference.

Design and construction of CRISPR-Cas9 plasmids for targeted gene editing of DAZL (deleted in azoospermia like) in PGC from chick embryos.

The Deleted in AZoospermia-Like (DAZL) (DAZL Locus on Chromosome 2: 34429592 . . . 34442888; GRCg6a) was selected as a target of genetic mutation for the production of sterile avian embryos (FIG. 1A). Mutations in this somatic gene result in both male and female sterility. The region of the gene in the area of exons 2-4 was targeted (FIG. 1A).

Specificity of the CRISPR-Cas9 system relies on the sgRNA sequence. For identifying the sgRNA sequences, three independent, online-available bioinformatics tools were used: https://design.synthego.com/#/, http://www.rgenome.net/, and http://www.e-crisp.org/E-CRISP/. These tools use different algorithms, thus cross comparing the results between them assists producing optimal results.

The criteria for choosing the sgRNA sequences were: 1. The sgRNA was identified within the highest ranking using the three algorithms; 2. The sgRNA target sequence was located in the vicinity of the beginning of the coding region in a shared exon; 3. Only 1 result with 0 mismatch (the sgRNA targets a unique site on the target DAZL gene); 4. There were no 1 or 2 mismatches predicted off-target sequences; and 5. Lowest number of 3 mismatch predicted off-targets, preferably in non-coding region. Essentially, the least number of potential mismatch sites, the better, when selecting the sgRNA. The algorithms were used to provide the number of putative off-target sites, and sgRNAs with the fewest chances of off-targets were selected. Searching under these criteria identified three (3) sgRNA sequences for the DAZL gene, as described in TABLE 3 below.

TABLE 3
sgRNA Sequences for DAZL Gene.

| sgRNA | CRISPR sgRNA | | | | |
|---|---|---|---|---|---|
| Sequence | sequence | | Genomic | | 3 mismatch |
| name | (SEQ ID NO:) | Strand | position | Targets | targets |
| CRISPR1 | CGGAGTTACTTTGAACAATA | — | Ch2- | 1 | 2 in non- |
| 1 | (SEQ ID NO: 1) | | 34437765 | | coding region |
| CRISPR2 | ACAATATGGTACTGTGAAGG | — | Ch2- | 1 | 0 |
| 2 | (SEQ ID NO: 2) | | 34437751 | | |
| CRISPR3 | TGAACAATATGGTACTGTGA | — | Ch2- | 1 | 3 in non- |
| 3 | (SEQ ID NO: 3) | | 34437754 | | coding region |
| | | | | | 1 in LRIT3 |

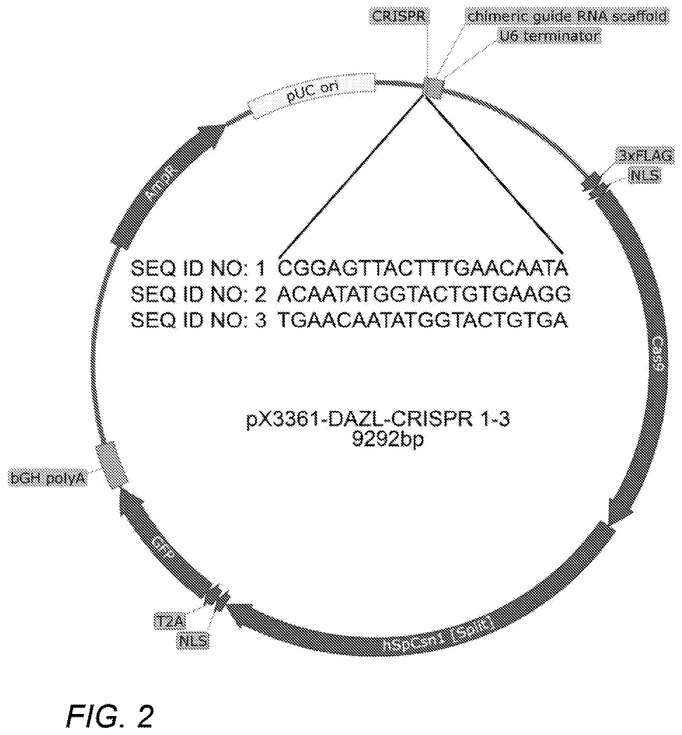

The CRISPR1, CRISPR2, and CRISPR3 sgRNA sequences were each cloned into the “all-in-one” px3361-GFP plasmid (modified from Addgene plasmid #42230, px330) (Mashiko et al. Feasibility for a large scale mouse mutagenesis by injecting CRISPR/Cas plasmid into zygotes. Devel. Growth Different. 56:122-129.) (see FIG. 2, showing the px3361-DAZL-CRISPR1 plasmid map), by cutting the plasmid with BbsI restriction enzyme. The digested plasmid serves as the backbone vector for CRISPR site insertion to form the sgRNA insert for ligation. This plasmid benefits from integrating the expression of the sgRNA and Cas9 fused in-frame to the GFP reporter gene and linked by the self-cleaving 2A peptide T2A, thus allowing for positive identification of transfected cells and single cell fluorescence-activated cell sorting (FACS) sorting. For ligation, the oligos for the sgRNA CRISPR sites—CRISPR1, CRISPR2 & CRISPR3 (oligos for CRISPR1 [forward (FWD)-caccgcggagttactttgaacaata (SEQ ID NO: 4); reverse (REV)-aaactattgttcaaagtaactccgc (SEQ ID NO: 5)]; for CRISPR2 [FWD-caccgacaatatggtactgtgaagg (SEQ ID NO: 6); REV-aaacccttcacagtaccatattgtc (SEQ ID NO: 7)]; for CRISPR3 [FWD-caccgtgaacaatatggtactgtga (SEQ ID NO: 8); REV-aaactcacagtaccatattgttcac (SEQ ID NO: 9)]) were denatured at 95° C. for 30 seconds, slowly annealed and ligated to the BbsI cut plasmids, transformed into E. coli, purified and sequence verified as described [mediadotaddgenedotorg/cms/filerpublic/e6/5a/e65a9ef8-c8ac-4B8-98da-3b7d7960394c/zhang-lab-general-cloning-protocoldotpdf; and Cong L, et al., Science. 2013 Jan. 3. 10.1126/science.1231143 PubMed 23287718]. Three expression vector plasmids: pX3361-DAZL-CRISPR1, pX3361-DAZL-CRISPR2, pX3361-DAZL-CRISPR3, were created. The plasmids were sequence verified and produced at the required concentration for expression in PGCs (1 μg/μl).

Expression vector plasmid pX3361-DAZL-CRISPR1 was found to have the sequence (SEQ ID NO: 10) shown in TABLE 4. The 20 bp CRISPR1 sgRNA sequence (CGGAGTTACTTTGAACAATA (SEQ ID NO: 1)) location is indicated (BOLD & Italics) at 251-270 bp. Expression vector plasmid pX3361-DAZL-CRISPR2 was found to have the sequence (SEQ ID NO: 33) shown in TABLE 4. The 20 bp CRISPR2 sgRNA sequence (ACAATATGGTACTGTGAAGG (SEQ ID NO: 2)) location is indicated (BOLD & Italics) at 251-270 bp. Expression vector plasmid pX3361-DAZL-CRISPR3 was found to have the sequence (SEQ ID NO: 34) shown in TABLE 4. The 20 bp CRISPR3 sgRNA sequence (TGAACAATATGGTACTGTGA (SEQ ID NO: 3)) location is indicated (BOLD & Italics) at 251-270 bp. FIG. 2 shows the plasmid map for pX3361-DAZL-CRISPR1, pX3361-DAZL-CRISPR2, and pX3361-DAZL-CRISPR3.

TABLE 4
Nucleotide Sequences of pX3361-DAZL-CRISPR1, pX3361-
DAZL-CRISPR2, and pX3361-DAZL-CRISPR3.

| Plasmid | Sequence |
|---|---|
| pX3361- | GAGGGCCTATTTCCCATGATTCCTTCATATTTGCATATACGAT |
| DAZL- | ACAAGGCTGTTAGAGAGATAATTGGAATTAATTTGACTGTAA |
| CRISPR1 | ACACAAAGATATTAGTACAAAATACGTGACGTAGAAAGTAA |
| | TAATTTCTTGGGTAGTTTGCAGTTTTAAAATTATGTTTTAAAA |
| | TGGACTATCATATGCTTACCGTAACTTGAAAGTATTTCGATTT |
| | CTTGGCTTTATATATCTTGTGGAAAGGACGAAACACCG [symbol] |
| | [symbol] [symbol] GTTTTAGAGCTAGAAATAGCAAGTTA |
| | AAATAAGGCTAGTCCGTTATCAACTTGAAAAAGTGGCACCGA |
| | GTCGGTGCTTTTTTGTTTTAGAGCTAGAAATAGCAAGTTAAA |
| | ATAAGGCTAGTCCGTTTTTAGCGCGTGCGCCAATTCTGCAGA |
| | CAAATGGCTCTAGAGGTACCCGTTACATAACTTACGGTAAAT |
| | GGCCCGCCTGGCTGACCGCCCAACGACCCCCGCCCATTGACG |
| | TCAATAGTAACGCCAATAGGGACTTTCCATTGACGTCAATGG |
| | GTGGAGTATTTACGGTAAACTGCCCACTTGGCAGTACATCAA |
| | GTGTATCATATGCCAAGTACGCCCCCTATTGACGTCAATGAC |
| | GGTAAATGGCCCGCCTGGCATTGTGCCCAGTACATGACCTTA |
| | TGGGACTTTCCTACTTGGCAGTACATCTACGTATTAGTCATCG |
| | CTATTACCATGGTCGAGGTGAGCCCCACGTTCTGCTTCACTCT |
| | CCCCATCTCCCCCCCCTCCCCACCCCCAATTTTGTATTTATTT |
| | ATTTTTTAATTATTTTGTGCAGCGATGGGGGCGGGGGGGGGG |
| | GGGGGGCGCGCGCCAGGCGGGGCGGGGCGGGGCGAGGGGC |
| | GGGGCGGGGCGAGGCGGAGAGGTGCGGCGGCAGCCAATCAG |
| | AGCGGCGCGCTCCGAAAGTTTCCTTTTATGGCGAGGCGGCGG |
| | CGGCGGCGGCCCTATAAAAAGCGAAGCGCGCGGCGGGCGGG |
| | AGTCGCTGCGACGCTGCCTTCGCCCCGTGCCCCGCTCCGCCG |
| | CCGCCTCGCGCCGCCCGCCCCGGCTCTGACTGACCGCGTTAC |
| | TCCCACAGGTGAGCGGGCGGGACGGCCCTTCTCCTCCGGGCT |
| | GTAATTAGCTGAGCAAGAGGTAAGGGTTTAAGGGATGGTTG |
| | GTTGGTGGGGTATTAATGTTTAATTACCTGGAGCACCTGCCT |
| | GAAATCACTTTTTTTCAGGTTGGACCGGTGCCACCATGGACT |
| | ATAAGGACCACGACGGAGACTACAAGGATCATGATATTGAT |
| | TACAAAGACGATGACGATAAGATGGCCCCAAAGAAGAAGCG |
| | GAAGGTCGGTATCCACGGAGTCCCAGCAGCCGACAAGAAGT |
| | ACAGCATCGGCCTGGACATCGGCACCAACTCTGTGGGCTGGG |
| | CCGTGATCACCGACGAGTACAAGGTGCCCAGCAAGAAATTC |
| | AAGGTGCTGGGCAACACCGACCGGCACAGCATCAAGAAGAA |
| | CCTGATCGGAGCCCTGCTGTTCGACAGCGGCGAAACAGCCGA |
| | GGCCACCCGGCTGAAGAGAACCGCCAGAAGAAGATACACCA |
| | GACGGAAGAACCGGATCTGCTATCTGCAAGAGATCTTCAGCA |
| | ACGAGATGGCCAAGGTGGACGACAGCTTCTTCCACAGACTG |
| | GAAGAGTCCTTCCTGGTGGAAGAGGATAAGAAGCACGAGCG |
| | GCACCCCATCTTCGGCAACATCGTGGACGAGGTGGCCTACCA |
| | CGAGAAGTACCCCACCATCTACCACCTGAGAAAGAAACTGG |
| | TGGACAGCACCGACAAGGCCGACCTGCGGCTGATCTATCTGG |
| | CCCTGGCCCACATGATCAAGTTCCGGGGCCACTTCCTGATCG |
| | AGGGCGACCTGAACCCCGACAACAGCGACGTGGACAAGCTG |
| | TTCATCCAGCTGGTGCAGACCTACAACCAGCTGTTCGAGGAA |
| | AACCCCATCAACGCCAGCGGCGTGGACGCCAAGGCCATCCT |
| | GTCTGCCAGACTGAGCAAGAGCAGACGGCTGGAAAATCTGA |
| | TCGCCCAGCTGCCCGGCGAGAAGAAGAATGGCCTGTTCGGA |
| | AACCTGATTGCCCTGAGCCTGGGCCTGACCCCCAACTTCAAG |
| | AGCAACTTCGACCTGGCCGAGGATGCCAAACTGCAGCTGAG |
| | CAAGGACACCTACGACGACGACCTGGACAACCTGCTGGCCC |
| | AGATCGGCGACCAGTACGCCGACCTGTTTCTGGCCGCCAAGA |
| | ACCTGTCCGACGCCATCCTGCTGAGCGACATCCTGAGAGTGA |
| | ACACCGAGATCACCAAGGCCCCCCTGAGCGCCTCTATGATCA |
| | AGAGATACGACGAGCACCACCAGGACCTGACCCTGCTGAAA |
| | GCTCTCGTGCGGCAGCAGCTGCCTGAGAAGTACAAAGAGATT |
| | TTCTTCGACCAGAGCAAGAACGGCTACGCCGGCTACATTGAC |
| | GGCGGAGCCAGCCAGGAAGAGTTCTACAAGTTCATCAAGCC |
| | CATCCTGGAAAAGATGGACGGCACCGAGGAACTGCTCGTGA |
| | AGCTGAACAGAGAGGACCTGCTGCGGAAGCAGCGGACCTTC |
| | GACAACGGCAGCATCCCCCACCAGATCCACCTGGGAGAGCT |
| | GCACGCCATTCTGCGGCGGCAGGAAGATTTTTACCCATTCCT |
| | GAAGGACAACCGGGAAAAGATCGAGAAGATCCTGACCTTCC |
| | GCATCCCCTACTACGTGGGCCCTCTGGCCAGGGGAAACAGCA |
| | GATTCGCCTGGATGACCAGAAAGAGCGAGGAAACCATCACC |
| | CCCTGGAACTTCGAGGAAGTGGTGGACAAGGGCGCTTCCGCC |
| | CAGAGCTTCATCGAGCGGATGACCAACTTCGATAAGAACCTG |
| | CCCAACGAGAAGGTGCTGCCCAAGCACAGCCTGCTGTACGA |
| | GTACTTCACCGTGTATAACGAGCTGACCAAAGTGAAATACGT |
| | GACCGAGGGAATGAGAAAGCCCGCCTTCCTGAGCGGCGAGC |
| | AGAAAAAGGCCATCGTGGACCTGCTGTTCAAGACCAACCGG |
| | AAAGTGACCGTGAAGCAGCTGAAAGAGGACTACTTCAAGAA |
| | AATCGAGTGCTTCGACTCCGTGGAAATCTCCGGCGTGGAAGA |
| | TCGGTTCAACGCCTCCCTGGGCACATACCACGATCTGCTGAA |
| | AATTATCAAGGACAAGGACTTCCTGGACAATGAGGAAAACG |
| | AGGACATTCTGGAAGATATCGTGCTGACCCTGACACTGTTTG |
| | AGGACAGAGAGATGATCGAGGAACGGCTGAAAACCTATGCC |
| | CACCTGTTCGACGACAAAGTGATGAAGCAGCTGAAGCGGCG |
| | GAGATACACCGGCTGGGGCAGGCTGAGCCGGAAGCTGATCA |
| | ACGGCATCCGGGACAAGCAGTCCGGCAAGACAATCCTGGAT |
| | TTCCTGAAGTCCGACGGCTTCGCCAACAGAAACTTCATGCAG |
| | CTGATCCACGACGACAGCCTGACCTTTAAAGAGGACATCCAG |
| | AAAGCCCAGGTGTCCGGCCAGGGCGATAGCCTGCACGAGCA |
| | CATTGCCAATCTGGCCGGCAGCCCCGCCATTAAGAAGGGCAT |
| | CCTGCAGACAGTGAAGGTGGTGGACGAGCTCGTGAAAGTGA |
| | TGGGCCGGCACAAGCCCGAGAACATCGTGATCGAAATGGCC |
| | AGAGAGAACCAGACCACCCAGAAGGGACAGAAGAACAGCC |
| | GCGAGAGAATGAAGCGGATCGAAGAGGGCATCAAAGAGCTG |
| | GGCAGCCAGATCCTGAAAGAACACCCCGTGGAAAACACCCA |
| | GCTGCAGAACGAGAAGCTGTACCTGTACTACCTGCAGAATGG |
| | GCGGGATATGTACGTGGACCAGGAACTGGACATCAACCGGC |
| | TGTCCGACTACGATGTGGACCATATCGTGCCTCAGAGCTTTC |
| | TGAAGGACGACTCCATCGACAACAAGGTGCTGACCAGAAGC |
| | GACAAGAACCGGGGCAAGAGCGACAACGTGCCCTCCGAAGA |
| | GGTCGTGAAGAAGATGAAGAACTACTGGCGGCAGCTGCTGA |
| | ACGCCAAGCTGATTACCCAGAGAAAGTTCGACAATCTGACCA |
| | AGGCCGAGAGAGGCGGCCTGAGCGAACTGGATAAGGCCGGC |
| | TTCATCAAGAGACAGCTGGTGGAAACCCGGCAGATCACAAA |
| | GCACGTGGCACAGATCCTGGACTCCCGGATGAACACTAAGTA |
| | CGACGAGAATGACAAGCTGATCCGGGAAGTGAAAGTGATCA |
| | CCCTGAAGTCCAAGCTGGTGTCCGATTTCCGGAAGGATTTCC |
| | AGTTTTACAAAGTGCGCGAGATCAACAACTACCACCACGCCC |
| | ACGACGCCTACCTGAACGCCGTCGTGGGAACCGCCCTGATCA |
| | AAAAGTACCCTAAGCTGGAAAGCGAGTTCGTGTACGGCGAC |
| | TACAAGGTGTACGACGTGCGGAAGATGATCGCCAAGAGCGA |
| | GCAGGAAATCGGCAAGGCTACCGCCAAGTACTTCTTCTACAG |
| | CAACATCATGAACTTTTTCAAGACCGAGATTACCCTGGCCAA |
| | CGGCGAGATCCGGAAGCGGCCTCTGATCGAGACAAACGGCG |
| | AAACCGGGGAGATCGTGTGGGATAAGGGCCGGGATTTTGCC |
| | ACCGTGCGGAAAGTGCTGAGCATGCCCCAAGTGAATATCGTG |
| | AAAAAGACCGAGGTGCAGACAGGCGGCTTCAGCAAAGAGTC |
| | TATCCTGCCCAAGAGGAACAGCGATAAGCTGATCGCCAGAA |
| | AGAAGGACTGGGACCCTAAGAAGTACGGCGGCTTCGACAGC |
| | CCCACCGTGGCCTATTCTGTGCTGGTGGTGGCCAAAGTGGAA |
| | AAGGGCAAGTCCAAGAAACTGAAGAGTGTGAAAGAGCTGCT |
| | GGGGATCACCATCATGGAAAGAAGCAGCTTCGAGAAGAATC |
| | CCATCGACTTTCTGGAAGCCAAGGGCTACAAAGAAGTGAAA |
| | AAGGACCTGATCATCAAGCTGCCTAAGTACTCCCTGTTCGAG |
| | CTGGAAAACGGCCGGAAGAGAATGCTGGCCTCTGCCGGCGA |
| | ACTGCAGAAGGGAAACGAACTGGCCCTGCCCTCCAAATATGT |
| | GAACTTCCTGTACCTGGCCAGCCACTATGAGAAGCTGAAGGG |
| | CTCCCCCGAGGATAATGAGCAGAAACAGCTGTTTGTGGAACA |
| | GCACAAGCACTACCTGGACGAGATCATCGAGCAGATCAGCG |
| | AGTTCTCCAAGAGAGTGATCCTGGCCGACGCTAATCTGGACA |
| | AAGTGCTGTCCGCCTACAACAAGCACCGGGATAAGCCCATCA |
| | GAGAGCAGGCCGAGAATATCATCCACCTGTTTACCCTGACCA |
| | ATCTGGGAGCCCCTGCCGCCTTCAAGTACTTTGACACCACCA |
| | TCGACCGGAAGAGGTACACCAGCACCAAAGAGGTGCTGGAC |
| | GCCACCCTGATCCACCAGAGCATCACCGGCCTGTACGAGACA |
| | CGGATCGACCTGTCTCAGCTGGGAGGCGACAAAAGGCCGGC |
| | GGCCACGAAAAAGGCCGGCCAGGCAAAAAAGAAAAAGGAA |
| | TTCGGCAGTGGAGAGGGCAGAGGAAGTCTGCTAACATGCGG |
| | TGACGTCGAGGAGAATCCTGGCCCAGTGAGCAAGGGCGAGG |
| | AGCTGTTCACCGGGGTGGTGCCCATCCTGGTCGAGCTGGACG |
| | GCGACGTAAACGGCCACAAGTTCAGCGTGTCCGGCGAGGGC |
| | GAGGGCGATGCCACCTACGGCAAGCTGACCCTGAAGTTCATC |
| | TGCACCACCGGCAAGCTGCCCGTGCCCTGGCCCACCCTCGTG |
| | ACCACCCTGACCTACGGCGTGCAGTGCTTCAGCCGCTACCCC |
| | GACCACATGAAGCAGCACGACTTCTTCAAGTCCGCCATGCCC |
| | GAAGGCTACGTCCAGGAGCGCACCATCTTCTTCAAGGACGAC |
| | GGCAACTACAAGACCCGCGCCGAGGTGAAGTTCGAGGGCGA |
| | CACCCTGGTGAACCGCATCGAGCTGAAGGGCATCGACTTCAA |
| | GGAGGACGGCAACATCCTGGGGCACAAGCTGGAGTACAACT |
| | ACAACAGCCACAACGTCTATATCATGGCCGACAAGCAGAAG |
| | AACGGCATCAAGGTGAACTTCAAGATCCGCCACAACATCGA |
| | GGACGGCAGCGTGCAGCTCGCCGACCACTACCAGCAGAACA |
| | CCCCCATCGGCGACGGCCCCGTGCTGCTGCCCGACAACCACT |
| | ACCTGAGCACCCAGTCCGCCCTGAGCAAAGACCCCAACGAG |
| | AAGCGCGATCACATGGTCCTGCTGGAGTTCGTGACCGCCGCC |
| | GGGATCACTCTCGGCATGGACGAGCTGTACAAGGAATTCTAA |
| | CTAGAGCTCGCTGATCAGCCTCGACTGTGCCTTCTAGTTGCC |
| | AGCCATCTGTTGTTTGCCCCTCCCCCGTGCCTTCCTTGACCCT |
| | GGAAGGTGCCACTCCCACTGTCCTTTCCTAATAAAATGAGGA |
| | AATTGCATCGCATTGTCTGAGTAGGTGTCATTCTATTCTGGGG |
| | GGTGGGGTGGGGCAGGACAGCAAGGGGGAGGATTGGGAAG |
| | AGAATAGCAGGCATGCTGGGGAGCGGCCGCAGGAACCCCTA |
| | GTGATGGAGTTGGCCACTCCCTCTCTGCGCGCTCGCTCGCTC |
| | ACTGAGGCCGGGCGACCAAAGGTCGCCCGACGCCCGGGCTT |
| | TGCCCGGGCGGCCTCAGTGAGCGAGCGAGCGCGCAGCTGCC |
| | TGCAGGGGCGCCTGATGCGGTATTTTCTCCTTACGCATCTGTG |
| | CGGTATTTCACACCGCATACGTCAAAGCAACCATAGTACGCG |
| | CCCTGTAGCGGCGCATTAAGCGCGGCGGGTGTGGTGGTTACG |
| | CGCAGCGTGACCGCTACACTTGCCAGCGCCCTAGCGCCCGCT |
| | CCTTTCGCTTTCTTCCCTTCCTTTCTCGCCACGTTCGCCGGCTT |
| | TCCCCGTCAAGCTCTAAATCGGGGGCTCCCTTTAGGGTTCCG |
| | ATTTAGTGCTTTACGGCACCTCGACCCCAAAAAACTTGATTT |
| | GGGTGATGGTTCACGTAGTGGGCCATCGCCCTGATAGACGGT |
| | TTTTCGCCCTTTGACGTTGGAGTCCACGTTCTTTAATAGTGGA |
| | CTCTTGTTCCAAACTGGAACAACACTCAACCCTATCTCGGGC |
| | TATTCTTTTGATTTATAAGGGATTTTGCCGATTTCGGCCTATT |
| | GGTTAAAAAATGAGCTGATTTAACAAAAATTTAACGCGAATT |
| | TTAACAAAATATTAACGTTTACAATTTTATGGTGCACTCTCAG |
| | TACAATCTGCTCTGATGCCGCATAGTTAAGCCAGCCCCGACA |
| | CCCGCCAACACCCGCTGACGCGCCCTGACGGGCTTGTCTGCT |
| | CCCGGCATCCGCTTACAGACAAGCTGTGACCGTCTCCGGGAG |
| | CTGCATGTGTCAGAGGTTTTCACCGTCATCACCGAAACGCGC |
| | GAGACGAAAGGGCCTCGTGATACGCCTATTTTTATAGGTTAA |
| | TGTCATGATAATAATGGTTTCTTAGACGTCAGGTGGCACTTTT |
| | CGGGGAAATGTGCGCGGAACCCCTATTTGTTTATTTTTCTAA |
| | ATACATTCAAATATGTATCCGCTCATGAGACAATAACCCTGA |
| | TAAATGCTTCAATAATATTGAAAAAGGAAGAGTATGAGTATT |
| | CAACATTTCCGTGTCGCCCTTATTCCCTTTTTTGCGGCATTTT |
| | GCCTTCCTGTTTTTGCTCACCCAGAAACGCTGGTGAAAGTAA |
| | AAGATGCTGAAGATCAGTTGGGTGCACGAGTGGGTTACATCG |
| | AACTGGATCTCAACAGCGGTAAGATCCTTGAGAGTTTTCGCC |
| | CCGAAGAACGTTTTCCAATGATGAGCACTTTTAAAGTTCTGC |
| | TATGTGGCGCGGTATTATCCCGTATTGACGCCGGGCAAGAGC |
| | AACTCGGTCGCCGCATACACTATTCTCAGAATGACTTGGTTG |
| | AGTACTCACCAGTCACAGAAAAGCATCTTACGGATGGCATGA |
| | CAGTAAGAGAATTATGCAGTGCTGCCATAACCATGAGTGATA |
| | ACACTGCGGCCAACTTACTTCTGACAACGATCGGAGGACCGA |
| | AGGAGCTAACCGCTTTTTTGCACAACATGGGGGATCATGTAA |
| | CTCGCCTTGATCGTTGGGAACCGGAGCTGAATGAAGCCATAC |
| | CAAACGACGAGCGTGACACCACGATGCCTGTAGCAATGGCA |
| | ACAACGTTGCGCAAACTATTAACTGGCGAACTACTTACTCTA |
| | GCTTCCCGGCAACAATTAATAGACTGGATGGAGGCGGATAA |
| | AGTTGCAGGACCACTTCTGCGCTCGGCCCTTCCGGCTGGCTG |
| | GTTTATTGCTGATAAATCTGGAGCCGGTGAGCGTGGAAGCCG |
| | CGGTATCATTGCAGCACTGGGGCCAGATGGTAAGCCCTCCCG |
| | TATCGTAGTTATCTACACGACGGGGAGTCAGGCAACTATGGA |
| | TGAACGAAATAGACAGATCGCTGAGATAGGTGCCTCACTGAT |
| | TAAGCATTGGTAACTGTCAGACCAAGTTTACTCATATATACT |
| | TTAGATTGATTTAAAACTTCATTTTTAATTTAAAAGGATCTAG |
| | GTGAAGATCCTTTTTGATAATCTCATGACCAAAATCCCTTAA |
| | CGTGAGTTTTCGTTCCACTGAGCGTCAGACCCCGTAGAAAAG |
| | ATCAAAGGATCTTCTTGAGATCCTTTTTTTCTGCGCGTAATCT |
| | GCTGCTTGCAAACAAAAAAACCACCGCTACCAGCGGTGGTTT |
| | GTTTGCCGGATCAAGAGCTACCAACTCTTTTTCCGAAGGTAA |
| | CTGGCTTCAGCAGAGCGCAGATACCAAATACTGTCCTTCTAG |
| | TGTAGCCGTAGTTAGGCCACCACTTCAAGAACTCTGTAGCAC |
| | CGCCTACATACCTCGCTCTGCTAATCCTGTTACCAGTGGCTGC |
| | TGCCAGTGGCGATAAGTCGTGTCTTACCGGGTTGGACTCAAG |
| | ACGATAGTTACCGGATAAGGCGCAGCGGTCGGGCTGAACGG |
| | GGGGTTCGTGCACACAGCCCAGCTTGGAGCGAACGACCTAC |
| | ACCGAACTGAGATACCTACAGCGTGAGCTATGAGAAAGCGC |
| | CACGCTTCCCGAAGGGAGAAAGGCGGACAGGTATCCGGTAA |
| | GCGGCAGGGTCGGAACAGGAGAGCGCACGAGGGAGCTTCCA |
| | GGGGGAAACGCCTGGTATCTTTATAGTCCTGTCGGGTTTCGC |
| | CACCTCTGACTTGAGCGTCGATTTTTGTGATGCTCGTCAGGG |
| | GGGCGGAGCCTATGGAAAAACGCCAGCAACGCGGCCTTTTT |
| | ACGGTTCCTGGCCTTTTGCTGGCCTTTTGCTCACATGT (SEQ ID |
| | NO: 10) |
| pX3361- | GAGGGCCTATTTCCCATGATTCCTTCATATTTGCATATACGAT |
| DAZL- | ACAAGGCTGTTAGAGAGATAATTGGAATTAATTTGACTGTAA |
| CRISPR2 | ACACAAAGATATTAGTACAAAATACGTGACGTAGAAAGTAAT |
| | AATTTCTTGGGTAGTTTGCAGTTTTAAAATTATGTTTTAAAAT |
| | GGACTATCATATGCTTACCGTAACTTGAAAGTATTTCGATTTC |
| | TTGGCTTTATATATCTTGTGGAAAGGACGAAACACCG [symbol] |
| | [symbol] GTTTTAGAGCTAGAAATAGCAAGTTAAA |
| | ATAAGGCTAGTCCGTTATCAACTTGAAAAAGTGGCACCGAGT |
| | CGGTGCTTTTTTGTTTTAGAGCTAGAAATAGCAAGTTAAAATA |
| | AGGCTAGTCCGTTTTTAGCGCGTGCGCCAATTCTGCAGACAAA |
| | TGGCTCTAGAGGTACCCGTTACATAACTTACGGTAAATGGCCC |
| | GCCTGGCTGACCGCCCAACGACCCCCGCCCATTGACGTCAAT |
| | AGTAACGCCAATAGGGACTTTCCATTGACGTCAATGGGTGGA |
| | GTATTTACGGTAAACTGCCCACTTGGCAGTACATCAAGTGTAT |
| | CATATGCCAAGTACGCCCCCTATTGACGTCAATGACGGTAAA |
| | TGGCCCGCCTGGCATTGTGCCCAGTACATGACCTTATGGGACT |
| | TTCCTACTTGGCAGTACATCTACGTATTAGTCATCGCTATTAC |
| | CATGGTCGAGGTGAGCCCCACGTTCTGCTTCACTCTCCCCATC |
| | TCCCCCCCCTCCCCACCCCCAATTTTGTATTTATTTATTTTTTA |
| | ATTATTTTGTGCAGCGATGGGGGCGGGGGGGGGGGGGGGGCG |
| | CGCGCCAGGCGGGGCGGGGCGGGGCGAGGGGCGGGGCGGGG |
| | CGAGGCGGAGAGGTGCGGCGGCAGCCAATCAGAGCGGCGCG |
| | CTCCGAAAGTTTCCTTTTATGGCGAGGCGGCGGCGGCGGCGG |
| | CCCTATAAAAAGCGAAGCGCGCGGCGGGCGGGAGTCGCTGCG |
| | ACGCTGCCTTCGCCCCGTGCCCCGCTCCGCCGCCGCCTCGCGC |
| | CGCCCGCCCCGGCTCTGACTGACCGCGTTACTCCCACAGGTGA |
| | GCGGGCGGGACGGCCCTTCTCCTCCGGGCTGTAATTAGCTGA |
| | GCAAGAGGTAAGGGTTTAAGGGATGGTTGGTTGGTGGGGTAT |
| | TAATGTTTAATTACCTGGAGCACCTGCCTGAAATCACTTTTTT |
| | TCAGGTTGGACCGGTGCCACCATGGACTATAAGGACCACGAC |
| | GGAGACTACAAGGATCATGATATTGATTACAAAGACGATGAC |
| | GATAAGATGGCCCCAAAGAAGAAGCGGAAGGTCGGTATCCA |
| | CGGAGTCCCAGCAGCCGACAAGAAGTACAGCATCGGCCTGGA |
| | CATCGGCACCAACTCTGTGGGCTGGGCCGTGATCACCGACGA |
| | GTACAAGGTGCCCAGCAAGAAATTCAAGGTGCTGGGCAACAC |
| | CGACCGGCACAGCATCAAGAAGAACCTGATCGGAGCCCTGCT |
| | GTTCGACAGCGGCGAAACAGCCGAGGCCACCCGGCTGAAGA |
| | GAACCGCCAGAAGAAGATACACCAGACGGAAGAACCGGATC |
| | TGCTATCTGCAAGAGATCTTCAGCAACGAGATGGCCAAGGTG |
| | GACGACAGCTTCTTCCACAGACTGGAAGAGTCCTTCCTGGTG |
| | GAAGAGGATAAGAAGCACGAGCGGCACCCCATCTTCGGCAAC |
| | ATCGTGGACGAGGTGGCCTACCACGAGAAGTACCCCACCATC |
| | TACCACCTGAGAAAGAAACTGGTGGACAGCACCGACAAGGCC |
| | GACCTGCGGCTGATCTATCTGGCCCTGGCCCACATGATCAAGT |
| | TCCGGGGCCACTTCCTGATCGAGGGCGACCTGAACCCCGACA |
| | ACAGCGACGTGGACAAGCTGTTCATCCAGCTGGTGCAGACCT |
| | ACAACCAGCTGTTCGAGGAAAACCCCATCAACGCCAGCGGCG |
| | TGGACGCCAAGGCCATCCTGTCTGCCAGACTGAGCAAGAGCA |
| | GACGGCTGGAAAATCTGATCGCCCAGCTGCCCGGCGAGAAGA |
| | AGAATGGCCTGTTCGGAAACCTGATTGCCCTGAGCCTGGGCC |
| | TGACCCCCAACTTCAAGAGCAACTTCGACCTGGCCGAGGATG |
| | CCAAACTGCAGCTGAGCAAGGACACCTACGACGACGACCTGG |
| | ACAACCTGCTGGCCCAGATCGGCGACCAGTACGCCGACCTGT |
| | TTCTGGCCGCCAAGAACCTGTCCGACGCCATCCTGCTGAGCG |
| | ACATCCTGAGAGTGAACACCGAGATCACCAAGGCCCCCCTGA |
| | GCGCCTCTATGATCAAGAGATACGACGAGCACCACCAGGACC |
| | TGACCCTGCTGAAAGCTCTCGTGCGGCAGCAGCTGCCTGAGA |
| | AGTACAAAGAGATTTTCTTCGACCAGAGCAAGAACGGCTACG |
| | CCGGCTACATTGACGGCGGAGCCAGCCAGGAAGAGTTCTACA |
| | AGTTCATCAAGCCCATCCTGGAAAAGATGGACGGCACCGAGG |
| | AACTGCTCGTGAAGCTGAACAGAGAGGACCTGCTGCGGAAGC |
| | AGCGGACCTTCGACAACGGCAGCATCCCCCACCAGATCCACC |
| | TGGGAGAGCTGCACGCCATTCTGCGGCGGCAGGAAGATTTTT |
| | ACCCATTCCTGAAGGACAACCGGGAAAAGATCGAGAAGATCC |
| | TGACCTTCCGCATCCCCTACTACGTGGGCCCTCTGGCCAGGGG |
| | AAACAGCAGATTCGCCTGGATGACCAGAAAGAGCGAGGAAA |
| | CCATCACCCCCTGGAACTTCGAGGAAGTGGTGGACAAGGGCG |
| | CTTCCGCCCAGAGCTTCATCGAGCGGATGACCAACTTCGATA |
| | AGAACCTGCCCAACGAGAAGGTGCTGCCCAAGCACAGCCTGC |
| | TGTACGAGTACTTCACCGTGTATAACGAGCTGACCAAAGTGA |
| | AATACGTGACCGAGGGAATGAGAAAGCCCGCCTTCCTGAGCG |
| | GCGAGCAGAAAAAGGCCATCGTGGACCTGCTGTTCAAGACCA |
| | ACCGGAAAGTGACCGTGAAGCAGCTGAAAGAGGACTACTTCA |
| | AGAAAATCGAGTGCTTCGACTCCGTGGAAATCTCCGGCGTGG |
| | AAGATCGGTTCAACGCCTCCCTGGGCACATACCACGATCTGCT |
| | GAAAATTATCAAGGACAAGGACTTCCTGGACAATGAGGAAAA |
| | CGAGGACATTCTGGAAGATATCGTGCTGACCCTGACACTGTTT |
| | GAGGACAGAGAGATGATCGAGGAACGGCTGAAAACCTATGC |
| | CCACCTGTTCGACGACAAAGTGATGAAGCAGCTGAAGCGGCG |
| | GAGATACACCGGCTGGGGCAGGCTGAGCCGGAAGCTGATCAA |
| | CGGCATCCGGGACAAGCAGTCCGGCAAGACAATCCTGGATTT |
| | CCTGAAGTCCGACGGCTTCGCCAACAGAAACTTCATGCAGCT |
| | GATCCACGACGACAGCCTGACCTTTAAAGAGGACATCCAGAA |
| | AGCCCAGGTGTCCGGCCAGGGCGATAGCCTGCACGAGCACAT |
| | TGCCAATCTGGCCGGCAGCCCCGCCATTAAGAAGGGCATCCT |
| | GCAGACAGTGAAGGTGGTGGACGAGCTCGTGAAAGTGATGG |
| | GCCGGCACAAGCCCGAGAACATCGTGATCGAAATGGCCAGAG |
| | AGAACCAGACCACCCAGAAGGGACAGAAGAACAGCCGCGAG |
| | AGAATGAAGCGGATCGAAGAGGGCATCAAAGAGCTGGGCAG |
| | CCAGATCCTGAAAGAACACCCCGTGGAAAACACCCAGCTGCA |
| | GAACGAGAAGCTGTACCTGTACTACCTGCAGAATGGGCGGGA |
| | TATGTACGTGGACCAGGAACTGGACATCAACCGGCTGTCCGA |
| | CTACGATGTGGACCATATCGTGCCTCAGAGCTTTCTGAAGGAC |
| | GACTCCATCGACAACAAGGTGCTGACCAGAAGCGACAAGAAC |
| | CGGGGCAAGAGCGACAACGTGCCCTCCGAAGAGGTCGTGAA |
| | GAAGATGAAGAACTACTGGCGGCAGCTGCTGAACGCCAAGCT |
| | GATTACCCAGAGAAAGTTCGACAATCTGACCAAGGCCGAGAG |
| | AGGCGGCCTGAGCGAACTGGATAAGGCCGGCTTCATCAAGAG |
| | ACAGCTGGTGGAAACCCGGCAGATCACAAAGCACGTGGCACA |
| | GATCCTGGACTCCCGGATGAACACTAAGTACGACGAGAATGA |
| | CAAGCTGATCCGGGAAGTGAAAGTGATCACCCTGAAGTCCAA |
| | GCTGGTGTCCGATTTCCGGAAGGATTTCCAGTTTTACAAAGTG |
| | CGCGAGATCAACAACTACCACCACGCCCACGACGCCTACCTG |
| | AACGCCGTCGTGGGAACCGCCCTGATCAAAAAGTACCCTAAG |
| | CTGGAAAGCGAGTTCGTGTACGGCGACTACAAGGTGTACGAC |
| | GTGCGGAAGATGATCGCCAAGAGCGAGCAGGAAATCGGCAA |
| | GGCTACCGCCAAGTACTTCTTCTACAGCAACATCATGAACTTT |
| | TTCAAGACCGAGATTACCCTGGCCAACGGCGAGATCCGGAAG |
| | CGGCCTCTGATCGAGACAAACGGCGAAACCGGGGAGATCGTG |
| | TGGGATAAGGGCCGGGATTTTGCCACCGTGCGGAAAGTGCTG |
| | AGCATGCCCCAAGTGAATATCGTGAAAAAGACCGAGGTGCAG |
| | ACAGGCGGCTTCAGCAAAGAGTCTATCCTGCCCAAGAGGAAC |
| | AGCGATAAGCTGATCGCCAGAAAGAAGGACTGGGACCCTAA |
| | GAAGTACGGCGGCTTCGACAGCCCCACCGTGGCCTATTCTGT |
| | GCTGGTGGTGGCCAAAGTGGAAAAGGGCAAGTCCAAGAAAC |
| | TGAAGAGTGTGAAAGAGCTGCTGGGGATCACCATCATGGAAA |
| | GAAGCAGCTTCGAGAAGAATCCCATCGACTTTCTGGAAGCCA |
| | AGGGCTACAAAGAAGTGAAAAAGGACCTGATCATCAAGCTGC |
| | CTAAGTACTCCCTGTTCGAGCTGGAAAACGGCCGGAAGAGAA |
| | TGCTGGCCTCTGCCGGCGAACTGCAGAAGGGAAACGAACTGG |
| | CCCTGCCCTCCAAATATGTGAACTTCCTGTACCTGGCCAGCCA |
| | CTATGAGAAGCTGAAGGGCTCCCCCGAGGATAATGAGCAGAA |
| | ACAGCTGTTTGTGGAACAGCACAAGCACTACCTGGACGAGAT |
| | CATCGAGCAGATCAGCGAGTTCTCCAAGAGAGTGATCCTGGC |
| | CGACGCTAATCTGGACAAAGTGCTGTCCGCCTACAACAAGCA |
| | CCGGGATAAGCCCATCAGAGAGCAGGCCGAGAATATCATCCA |
| | CCTGTTTACCCTGACCAATCTGGGAGCCCCTGCCGCCTTCAAG |
| | TACTTTGACACCACCATCGACCGGAAGAGGTACACCAGCACC |
| | AAAGAGGTGCTGGACGCCACCCTGATCCACCAGAGCATCACC |
| | GGCCTGTACGAGACACGGATCGACCTGTCTCAGCTGGGAGGC |
| | GACAAAAGGCCGGCGGCCACGAAAAAGGCCGGCCAGGCAAA |
| | AAAGAAAAAGGAATTCGGCAGTGGAGAGGGCAGAGGAAGTC |
| | TGCTAACATGCGGTGACGTCGAGGAGAATCCTGGCCCAGTGA |
| | GCAAGGGCGAGGAGCTGTTCACCGGGGTGGTGCCCATCCTGG |
| | TCGAGCTGGACGGCGACGTAAACGGCCACAAGTTCAGCGTGT |
| | CCGGCGAGGGCGAGGGCGATGCCACCTACGGCAAGCTGACCC |
| | TGAAGTTCATCTGCACCACCGGCAAGCTGCCCGTGCCCTGGCC |
| | CACCCTCGTGACCACCCTGACCTACGGCGTGCAGTGCTTCAGC |
| | CGCTACCCCGACCACATGAAGCAGCACGACTTCTTCAAGTCC |
| | GCCATGCCCGAAGGCTACGTCCAGGAGCGCACCATCTTCTTC |
| | AAGGACGACGGCAACTACAAGACCCGCGCCGAGGTGAAGTTC |
| | GAGGGCGACACCCTGGTGAACCGCATCGAGCTGAAGGGCATC |
| | GACTTCAAGGAGGACGGCAACATCCTGGGGCACAAGCTGGAG |
| | TACAACTACAACAGCCACAACGTCTATATCATGGCCGACAAG |
| | CAGAAGAACGGCATCAAGGTGAACTTCAAGATCCGCCACAAC |
| | ATCGAGGACGGCAGCGTGCAGCTCGCCGACCACTACCAGCAG |
| | AACACCCCCATCGGCGACGGCCCCGTGCTGCTGCCCGACAAC |
| | CACTACCTGAGCACCCAGTCCGCCCTGAGCAAAGACCCCAAC |
| | GAGAAGCGCGATCACATGGTCCTGCTGGAGTTCGTGACCGCC |
| | GCCGGGATCACTCTCGGCATGGACGAGCTGTACAAGGAATTC |
| | TAACTAGAGCTCGCTGATCAGCCTCGACTGTGCCTTCTAGTTG |
| | CCAGCCATCTGTTGTTTGCCCCTCCCCCGTGCCTTCCTTGACCC |
| | TGGAAGGTGCCACTCCCACTGTCCTTTCCTAATAAAATGAGGA |
| | AATTGCATCGCATTGTCTGAGTAGGTGTCATTCTATTCTGGGG |
| | GGTGGGGTGGGGCAGGACAGCAAGGGGGAGGATTGGGAAGA |
| | GAATAGCAGGCATGCTGGGGAGCGGCCGCAGGAACCCCTAGT |
| | GATGGAGTTGGCCACTCCCTCTCTGCGCGCTCGCTCGCTCACT |
| | GAGGCCGGGCGACCAAAGGTCGCCCGACGCCCGGGCTTTGCC |
| | CGGGCGGCCTCAGTGAGCGAGCGAGCGCGCAGCTGCCTGCAG |
| | GGGCGCCTGATGCGGTATTTTCTCCTTACGCATCTGTGCGGTA |
| | TTTCACACCGCATACGTCAAAGCAACCATAGTACGCGCCCTGT |
| | AGCGGCGCATTAAGCGCGGCGGGTGTGGTGGTTACGCGCAGC |
| | GTGACCGCTACACTTGCCAGCGCCCTAGCGCCCGCTCCTTTCG |
| | CTTTCTTCCCTTCCTTTCTCGCCACGTTCGCCGGCTTTCCCCGT |
| | CAAGCTCTAAATCGGGGGCTCCCTTTAGGGTTCCGATTTAGTG |
| | CTTTACGGCACCTCGACCCCAAAAAACTTGATTTGGGTGATGG |
| | TTCACGTAGTGGGCCATCGCCCTGATAGACGGTTTTTCGCCCT |
| | TTGACGTTGGAGTCCACGTTCTTTAATAGTGGACTCTTGTTCC |
| | AAACTGGAACAACACTCAACCCTATCTCGGGCTATTCTTTTGA |
| | TTTATAAGGGATTTTGCCGATTTCGGCCTATTGGTTAAAAAAT |
| | GAGCTGATTTAACAAAAATTTAACGCGAATTTTAACAAAATA |
| | TTAACGTTTACAATTTTATGGTGCACTCTCAGTACAATCTGCT |
| | CTGATGCCGCATAGTTAAGCCAGCCCCGACACCCGCCAACAC |
| | CCGCTGACGCGCCCTGACGGGCTTGTCTGCTCCCGGCATCCGC |
| | TTACAGACAAGCTGTGACCGTCTCCGGGAGCTGCATGTGTCA |
| | GAGGTTTTCACCGTCATCACCGAAACGCGCGAGACGAAAGGG |
| | CCTCGTGATACGCCTATTTTTATAGGTTAATGTCATGATAATA |
| | ATGGTTTCTTAGACGTCAGGTGGCACTTTTCGGGGAAATGTGC |
| | GCGGAACCCCTATTTGTTTATTTTTCTAAATACATTCAAATAT |
| | GTATCCGCTCATGAGACAATAACCCTGATAAATGCTTCAATA |
| | ATATTGAAAAAGGAAGAGTATGAGTATTCAACATTTCCGTGT |
| | CGCCCTTATTCCCTTTTTTGCGGCATTTTGCCTTCCTGTTTTTG |
| | CTCACCCAGAAACGCTGGTGAAAGTAAAAGATGCTGAAGATC |
| | AGTTGGGTGCACGAGTGGGTTACATCGAACTGGATCTCAACA |
| | GCGGTAAGATCCTTGAGAGTTTTCGCCCCGAAGAACGTTTTCC |
| | AATGATGAGCACTTTTAAAGTTCTGCTATGTGGCGCGGTATTA |
| | TCCCGTATTGACGCCGGGCAAGAGCAACTCGGTCGCCGCATA |
| | CACTATTCTCAGAATGACTTGGTTGAGTACTCACCAGTCACAG |
| | AAAAGCATCTTACGGATGGCATGACAGTAAGAGAATTATGCA |
| | GTGCTGCCATAACCATGAGTGATAACACTGCGGCCAACTTAC |
| | TTCTGACAACGATCGGAGGACCGAAGGAGCTAACCGCTTTTT |
| | TGCACAACATGGGGGATCATGTAACTCGCCTTGATCGTTGGG |
| | AACCGGAGCTGAATGAAGCCATACCAAACGACGAGCGTGAC |
| | ACCACGATGCCTGTAGCAATGGCAACAACGTTGCGCAAACTA |
| | TTAACTGGCGAACTACTTACTCTAGCTTCCCGGCAACAATTAA |
| | TAGACTGGATGGAGGCGGATAAAGTTGCAGGACCACTTCTGC |
| | GCTCGGCCCTTCCGGCTGGCTGGTTTATTGCTGATAAATCTGG |
| | AGCCGGTGAGCGTGGAAGCCGCGGTATCATTGCAGCACTGGG |
| | GCCAGATGGTAAGCCCTCCCGTATCGTAGTTATCTACACGACG |
| | GGGAGTCAGGCAACTATGGATGAACGAAATAGACAGATCGCT |
| | GAGATAGGTGCCTCACTGATTAAGCATTGGTAACTGTCAGAC |
| | CAAGTTTACTCATATATACTTTAGATTGATTTAAAACTTCATTT |
| | TTAATTTAAAAGGATCTAGGTGAAGATCCTTTTTGATAATCTC |
| | ATGACCAAAATCCCTTAACGTGAGTTTTCGTTCCACTGAGCGT |
| | CAGACCCCGTAGAAAAGATCAAAGGATCTTCTTGAGATCCTT |
| | TTTTTCTGCGCGTAATCTGCTGCTTGCAAACAAAAAAACCACC |
| | GCTACCAGCGGTGGTTTGTTTGCCGGATCAAGAGCTACCAACT |
| | CTTTTTCCGAAGGTAACTGGCTTCAGCAGAGCGCAGATACCA |
| | AATACTGTCCTTCTAGTGTAGCCGTAGTTAGGCCACCACTTCA |
| | AGAACTCTGTAGCACCGCCTACATACCTCGCTCTGCTAATCCT |
| | GTTACCAGTGGCTGCTGCCAGTGGCGATAAGTCGTGTCTTACC |
| | GGGTTGGACTCAAGACGATAGTTACCGGATAAGGCGCAGCGG |
| | TCGGGCTGAACGGGGGGTTCGTGCACACAGCCCAGCTTGGAG |
| | CGAACGACCTACACCGAACTGAGATACCTACAGCGTGAGCTA |
| | TGAGAAAGCGCCACGCTTCCCGAAGGGAGAAAGGCGGACAG |
| | GTATCCGGTAAGCGGCAGGGTCGGAACAGGAGAGCGCACGA |
| | GGGAGCTTCCAGGGGGAAACGCCTGGTATCTTTATAGTCCTGT |
| | CGGGTTTCGCCACCTCTGACTTGAGCGTCGATTTTTGTGATGC |
| | TCGTCAGGGGGGCGGAGCCTATGGAAAAACGCCAGCAACGC |
| | GGCCTTTTTACGGTTCCTGGCCTTTTGCTGGCCTTTTGCTCACA |
| | TGT (SEQ ID NO: 33) |
| pX3361- | GAGGGCCTATTTCCCATGATTCCTTCATATTTGCATATACGAT |
| DAZL- | ACAAGGCTGTTAGAGAGATAATTGGAATTAATTTGACTGTAA |
| CRISPR3 | ACACAAAGATATTAGTACAAAATACGTGACGTAGAAAGTAAT |
| | AATTTCTTGGGTAGTTTGCAGTTTTAAAATTATGTTTTAAAAT |
| | GGACTATCATATGCTTACCGTAACTTGAAAGTATTTCGATTTC |
| | TTGGCTTTATATATCTTGTGGAAAGGACGAAACACCG [symbol] |
| | [symbol] GTTTTAGAGCTAGAAATAGCAAGTTAAAA |
| | TAAGGCTAGTCCGTTATCAACTTGAAAAAGTGGCACCGAGTC |
| | GGTGCTTTTTTGTTTTAGAGCTAGAAATAGCAAGTTAAAATAA |
| | GGCTAGTCCGTTTTTAGCGCGTGCGCCAATTCTGCAGACAAAT |
| | GGCTCTAGAGGTACCCGTTACATAACTTACGGTAAATGGCCC |
| | GCCTGGCTGACCGCCCAACGACCCCCGCCCATTGACGTCAAT |
| | AGTAACGCCAATAGGGACTTTCCATTGACGTCAATGGGTGGA |
| | GTATTTACGGTAAACTGCCCACTTGGCAGTACATCAAGTGTAT |
| | CATATGCCAAGTACGCCCCCTATTGACGTCAATGACGGTAAA |
| | TGGCCCGCCTGGCATTGTGCCCAGTACATGACCTTATGGGACT |
| | TTCCTACTTGGCAGTACATCTACGTATTAGTCATCGCTATTAC |
| | CATGGTCGAGGTGAGCCCCACGTTCTGCTTCACTCTCCCCATC |
| | TCCCCCCCCTCCCCACCCCCAATTTTGTATTTATTTATTTTTTA |
| | ATTATTTTGTGCAGCGATGGGGGCGGGGGGGGGGGGGGGGCG |
| | CGCGCCAGGCGGGGCGGGGCGGGGCGAGGGGCGGGGCGGGG |
| | CGAGGCGGAGAGGTGCGGCGGCAGCCAATCAGAGCGGCGCG |
| | CTCCGAAAGTTTCCTTTTATGGCGAGGCGGCGGCGGCGGCGG |
| | CCCTATAAAAAGCGAAGCGCGCGGCGGGCGGGAGTCGCTGCG |
| | ACGCTGCCTTCGCCCCGTGCCCCGCTCCGCCGCCGCCTCGCGC |
| | CGCCCGCCCCGGCTCTGACTGACCGCGTTACTCCCACAGGTGA |
| | GCGGGCGGGACGGCCCTTCTCCTCCGGGCTGTAATTAGCTGA |
| | GCAAGAGGTAAGGGTTTAAGGGATGGTTGGTTGGTGGGGTAT |
| | TAATGTTTAATTACCTGGAGCACCTGCCTGAAATCACTTTTTT |
| | TCAGGTTGGACCGGTGCCACCATGGACTATAAGGACCACGAC |
| | GGAGACTACAAGGATCATGATATTGATTACAAAGACGATGAC |
| | GATAAGATGGCCCCAAAGAAGAAGCGGAAGGTCGGTATCCA |
| | CGGAGTCCCAGCAGCCGACAAGAAGTACAGCATCGGCCTGGA |
| | CATCGGCACCAACTCTGTGGGCTGGGCCGTGATCACCGACGA |
| | GTACAAGGTGCCCAGCAAGAAATTCAAGGTGCTGGGCAACAC |
| | CGACCGGCACAGCATCAAGAAGAACCTGATCGGAGCCCTGCT |
| | GTTCGACAGCGGCGAAACAGCCGAGGCCACCCGGCTGAAGA |
| | GAACCGCCAGAAGAAGATACACCAGACGGAAGAACCGGATC |
| | TGCTATCTGCAAGAGATCTTCAGCAACGAGATGGCCAAGGTG |
| | GACGACAGCTTCTTCCACAGACTGGAAGAGTCCTTCCTGGTG |
| | GAAGAGGATAAGAAGCACGAGCGGCACCCCATCTTCGGCAAC |
| | ATCGTGGACGAGGTGGCCTACCACGAGAAGTACCCCACCATC |
| | TACCACCTGAGAAAGAAACTGGTGGACAGCACCGACAAGGCC |
| | GACCTGCGGCTGATCTATCTGGCCCTGGCCCACATGATCAAGT |
| | TCCGGGGCCACTTCCTGATCGAGGGCGACCTGAACCCCGACA |
| | ACAGCGACGTGGACAAGCTGTTCATCCAGCTGGTGCAGACCT |
| | ACAACCAGCTGTTCGAGGAAAACCCCATCAACGCCAGCGGCG |
| | TGGACGCCAAGGCCATCCTGTCTGCCAGACTGAGCAAGAGCA |
| | GACGGCTGGAAAATCTGATCGCCCAGCTGCCCGGCGAGAAGA |
| | AGAATGGCCTGTTCGGAAACCTGATTGCCCTGAGCCTGGGCC |
| | TGACCCCCAACTTCAAGAGCAACTTCGACCTGGCCGAGGATG |
| | CCAAACTGCAGCTGAGCAAGGACACCTACGACGACGACCTGG |
| | ACAACCTGCTGGCCCAGATCGGCGACCAGTACGCCGACCTGT |
| | TTCTGGCCGCCAAGAACCTGTCCGACGCCATCCTGCTGAGCG |
| | ACATCCTGAGAGTGAACACCGAGATCACCAAGGCCCCCCTGA |
| | GCGCCTCTATGATCAAGAGATACGACGAGCACCACCAGGACC |
| | TGACCCTGCTGAAAGCTCTCGTGCGGCAGCAGCTGCCTGAGA |
| | AGTACAAAGAGATTTTCTTCGACCAGAGCAAGAACGGCTACG |
| | CCGGCTACATTGACGGCGGAGCCAGCCAGGAAGAGTTCTACA |
| | AGTTCATCAAGCCCATCCTGGAAAAGATGGACGGCACCGAGG |
| | AACTGCTCGTGAAGCTGAACAGAGAGGACCTGCTGCGGAAGC |
| | AGCGGACCTTCGACAACGGCAGCATCCCCCACCAGATCCACC |
| | TGGGAGAGCTGCACGCCATTCTGCGGCGGCAGGAAGATTTTT |
| | ACCCATTCCTGAAGGACAACCGGGAAAAGATCGAGAAGATCC |
| | TGACCTTCCGCATCCCCTACTACGTGGGCCCTCTGGCCAGGGG |
| | AAACAGCAGATTCGCCTGGATGACCAGAAAGAGCGAGGAAA |
| | CCATCACCCCCTGGAACTTCGAGGAAGTGGTGGACAAGGGCG |
| | CTTCCGCCCAGAGCTTCATCGAGCGGATGACCAACTTCGATA |
| | AGAACCTGCCCAACGAGAAGGTGCTGCCCAAGCACAGCCTGC |
| | TGTACGAGTACTTCACCGTGTATAACGAGCTGACCAAAGTGA |
| | AATACGTGACCGAGGGAATGAGAAAGCCCGCCTTCCTGAGCG |
| | GCGAGCAGAAAAAGGCCATCGTGGACCTGCTGTTCAAGACCA |
| | ACCGGAAAGTGACCGTGAAGCAGCTGAAAGAGGACTACTTCA |
| | AGAAAATCGAGTGCTTCGACTCCGTGGAAATCTCCGGCGTGG |
| | AAGATCGGTTCAACGCCTCCCTGGGCACATACCACGATCTGCT |
| | GAAAATTATCAAGGACAAGGACTTCCTGGACAATGAGGAAAA |
| | CGAGGACATTCTGGAAGATATCGTGCTGACCCTGACACTGTTT |
| | GAGGACAGAGAGATGATCGAGGAACGGCTGAAAACCTATGC |
| | CCACCTGTTCGACGACAAAGTGATGAAGCAGCTGAAGCGGCG |
| | GAGATACACCGGCTGGGGCAGGCTGAGCCGGAAGCTGATCAA |
| | CGGCATCCGGGACAAGCAGTCCGGCAAGACAATCCTGGATTT |
| | CCTGAAGTCCGACGGCTTCGCCAACAGAAACTTCATGCAGCT |
| | GATCCACGACGACAGCCTGACCTTTAAAGAGGACATCCAGAA |
| | AGCCCAGGTGTCCGGCCAGGGCGATAGCCTGCACGAGCACAT |
| | TGCCAATCTGGCCGGCAGCCCCGCCATTAAGAAGGGCATCCT |
| | GCAGACAGTGAAGGTGGTGGACGAGCTCGTGAAAGTGATGG |
| | GCCGGCACAAGCCCGAGAACATCGTGATCGAAATGGCCAGAG |
| | AGAACCAGACCACCCAGAAGGGACAGAAGAACAGCCGCGAG |
| | AGAATGAAGCGGATCGAAGAGGGCATCAAAGAGCTGGGCAG |
| | CCAGATCCTGAAAGAACACCCCGTGGAAAACACCCAGCTGCA |
| | GAACGAGAAGCTGTACCTGTACTACCTGCAGAATGGGCGGGA |
| | TATGTACGTGGACCAGGAACTGGACATCAACCGGCTGTCCGA |
| | CTACGATGTGGACCATATCGTGCCTCAGAGCTTTCTGAAGGAC |
| | GACTCCATCGACAACAAGGTGCTGACCAGAAGCGACAAGAAC |
| | CGGGGCAAGAGCGACAACGTGCCCTCCGAAGAGGTCGTGAA |
| | GAAGATGAAGAACTACTGGCGGCAGCTGCTGAACGCCAAGCT |
| | GATTACCCAGAGAAAGTTCGACAATCTGACCAAGGCCGAGAG |
| | AGGCGGCCTGAGCGAACTGGATAAGGCCGGCTTCATCAAGAG |
| | ACAGCTGGTGGAAACCCGGCAGATCACAAAGCACGTGGCACA |
| | GATCCTGGACTCCCGGATGAACACTAAGTACGACGAGAATGA |
| | CAAGCTGATCCGGGAAGTGAAAGTGATCACCCTGAAGTCCAA |
| | GCTGGTGTCCGATTTCCGGAAGGATTTCCAGTTTTACAAAGTG |
| | CGCGAGATCAACAACTACCACCACGCCCACGACGCCTACCTG |
| | AACGCCGTCGTGGGAACCGCCCTGATCAAAAAGTACCCTAAG |
| | CTGGAAAGCGAGTTCGTGTACGGCGACTACAAGGTGTACGAC |
| | GTGCGGAAGATGATCGCCAAGAGCGAGCAGGAAATCGGCAA |
| | GGCTACCGCCAAGTACTTCTTCTACAGCAACATCATGAACTTT |
| | TTCAAGACCGAGATTACCCTGGCCAACGGCGAGATCCGGAAG |
| | CGGCCTCTGATCGAGACAAACGGCGAAACCGGGGAGATCGTG |
| | TGGGATAAGGGCCGGGATTTTGCCACCGTGCGGAAAGTGCTG |
| | AGCATGCCCCAAGTGAATATCGTGAAAAAGACCGAGGTGCAG |
| | ACAGGCGGCTTCAGCAAAGAGTCTATCCTGCCCAAGAGGAAC |
| | AGCGATAAGCTGATCGCCAGAAAGAAGGACTGGGACCCTAA |
| | GAAGTACGGCGGCTTCGACAGCCCCACCGTGGCCTATTCTGT |
| | GCTGGTGGTGGCCAAAGTGGAAAAGGGCAAGTCCAAGAAAC |
| | TGAAGAGTGTGAAAGAGCTGCTGGGGATCACCATCATGGAAA |
| | GAAGCAGCTTCGAGAAGAATCCCATCGACTTTCTGGAAGCCA |
| | AGGGCTACAAAGAAGTGAAAAAGGACCTGATCATCAAGCTGC |
| | CTAAGTACTCCCTGTTCGAGCTGGAAAACGGCCGGAAGAGAA |
| | TGCTGGCCTCTGCCGGCGAACTGCAGAAGGGAAACGAACTGG |
| | CCCTGCCCTCCAAATATGTGAACTTCCTGTACCTGGCCAGCCA |
| | CTATGAGAAGCTGAAGGGCTCCCCCGAGGATAATGAGCAGAA |
| | ACAGCTGTTTGTGGAACAGCACAAGCACTACCTGGACGAGAT |
| | CATCGAGCAGATCAGCGAGTTCTCCAAGAGAGTGATCCTGGC |
| | CGACGCTAATCTGGACAAAGTGCTGTCCGCCTACAACAAGCA |
| | CCGGGATAAGCCCATCAGAGAGCAGGCCGAGAATATCATCCA |
| | CCTGTTTACCCTGACCAATCTGGGAGCCCCTGCCGCCTTCAAG |
| | TACTTTGACACCACCATCGACCGGAAGAGGTACACCAGCACC |
| | AAAGAGGTGCTGGACGCCACCCTGATCCACCAGAGCATCACC |
| | GGCCTGTACGAGACACGGATCGACCTGTCTCAGCTGGGAGGC |
| | GACAAAAGGCCGGCGGCCACGAAAAAGGCCGGCCAGGCAAA |
| | AAAGAAAAAGGAATTCGGCAGTGGAGAGGGCAGAGGAAGTC |
| | TGCTAACATGCGGTGACGTCGAGGAGAATCCTGGCCCAGTGA |
| | GCAAGGGCGAGGAGCTGTTCACCGGGGTGGTGCCCATCCTGG |
| | TCGAGCTGGACGGCGACGTAAACGGCCACAAGTTCAGCGTGT |
| | CCGGCGAGGGCGAGGGCGATGCCACCTACGGCAAGCTGACCC |
| | TGAAGTTCATCTGCACCACCGGCAAGCTGCCCGTGCCCTGGCC |
| | CACCCTCGTGACCACCCTGACCTACGGCGTGCAGTGCTTCAGC |
| | CGCTACCCCGACCACATGAAGCAGCACGACTTCTTCAAGTCC |
| | GCCATGCCCGAAGGCTACGTCCAGGAGCGCACCATCTTCTTC |
| | AAGGACGACGGCAACTACAAGACCCGCGCCGAGGTGAAGTTC |
| | GAGGGCGACACCCTGGTGAACCGCATCGAGCTGAAGGGCATC |
| | GACTTCAAGGAGGACGGCAACATCCTGGGGCACAAGCTGGAG |
| | TACAACTACAACAGCCACAACGTCTATATCATGGCCGACAAG |
| | CAGAAGAACGGCATCAAGGTGAACTTCAAGATCCGCCACAAC |
| | ATCGAGGACGGCAGCGTGCAGCTCGCCGACCACTACCAGCAG |
| | AACACCCCCATCGGCGACGGCCCCGTGCTGCTGCCCGACAAC |
| | CACTACCTGAGCACCCAGTCCGCCCTGAGCAAAGACCCCAAC |
| | GAGAAGCGCGATCACATGGTCCTGCTGGAGTTCGTGACCGCC |
| | GCCGGGATCACTCTCGGCATGGACGAGCTGTACAAGGAATTC |
| | TAACTAGAGCTCGCTGATCAGCCTCGACTGTGCCTTCTAGTTG |
| | CCAGCCATCTGTTGTTTGCCCCTCCCCCGTGCCTTCCTTGACCC |
| | TGGAAGGTGCCACTCCCACTGTCCTTTCCTAATAAAATGAGGA |
| | AATTGCATCGCATTGTCTGAGTAGGTGTCATTCTATTCTGGGG |
| | GGTGGGGTGGGGCAGGACAGCAAGGGGGAGGATTGGGAAGA |
| | GAATAGCAGGCATGCTGGGGAGCGGCCGCAGGAACCCCTAGT |
| | GATGGAGTTGGCCACTCCCTCTCTGCGCGCTCGCTCGCTCACT |
| | GAGGCCGGGCGACCAAAGGTCGCCCGACGCCCGGGCTTTGCC |
| | CGGGCGGCCTCAGTGAGCGAGCGAGCGCGCAGCTGCCTGCAG |
| | GGGCGCCTGATGCGGTATTTTCTCCTTACGCATCTGTGCGGTA |
| | TTTCACACCGCATACGTCAAAGCAACCATAGTACGCGCCCTGT |
| | AGCGGCGCATTAAGCGCGGCGGGTGTGGTGGTTACGCGCAGC |
| | GTGACCGCTACACTTGCCAGCGCCCTAGCGCCCGCTCCTTTCG |
| | CTTTCTTCCCTTCCTTTCTCGCCACGTTCGCCGGCTTTCCCCGT |
| | CAAGCTCTAAATCGGGGGCTCCCTTTAGGGTTCCGATTTAGTG |
| | CTTTACGGCACCTCGACCCCAAAAAACTTGATTTGGGTGATGG |
| | TTCACGTAGTGGGCCATCGCCCTGATAGACGGTTTTTCGCCCT |
| | TTGACGTTGGAGTCCACGTTCTTTAATAGTGGACTCTTGTTCC |
| | AAACTGGAACAACACTCAACCCTATCTCGGGCTATTCTTTTGA |
| | TTTATAAGGGATTTTGCCGATTTCGGCCTATTGGTTAAAAAAT |
| | GAGCTGATTTAACAAAAATTTAACGCGAATTTTAACAAAATA |
| | TTAACGTTTACAATTTTATGGTGCACTCTCAGTACAATCTGCT |
| | CTGATGCCGCATAGTTAAGCCAGCCCCGACACCCGCCAACAC |
| | CCGCTGACGCGCCCTGACGGGCTTGTCTGCTCCCGGCATCCGC |
| | TTACAGACAAGCTGTGACCGTCTCCGGGAGCTGCATGTGTCA |
| | GAGGTTTTCACCGTCATCACCGAAACGCGCGAGACGAAAGGG |
| | CCTCGTGATACGCCTATTTTTATAGGTTAATGTCATGATAATA |
| | ATGGTTTCTTAGACGTCAGGTGGCACTTTTCGGGGAAATGTGC |
| | GCGGAACCCCTATTTGTTTATTTTTCTAAATACATTCAAATAT |
| | GTATCCGCTCATGAGACAATAACCCTGATAAATGCTTCAATA |
| | ATATTGAAAAAGGAAGAGTATGAGTATTCAACATTTCCGTGT |
| | CGCCCTTATTCCCTTTTTTGCGGCATTTTGCCTTCCTGTTTTTG |
| | CTCACCCAGAAACGCTGGTGAAAGTAAAAGATGCTGAAGATC |
| | AGTTGGGTGCACGAGTGGGTTACATCGAACTGGATCTCAACA |
| | GCGGTAAGATCCTTGAGAGTTTTCGCCCCGAAGAACGTTTTCC |
| | AATGATGAGCACTTTTAAAGTTCTGCTATGTGGCGCGGTATTA |
| | TCCCGTATTGACGCCGGGCAAGAGCAACTCGGTCGCCGCATA |
| | CACTATTCTCAGAATGACTTGGTTGAGTACTCACCAGTCACAG |
| | AAAAGCATCTTACGGATGGCATGACAGTAAGAGAATTATGCA |
| | GTGCTGCCATAACCATGAGTGATAACACTGCGGCCAACTTAC |
| | TTCTGACAACGATCGGAGGACCGAAGGAGCTAACCGCTTTTT |
| | TGCACAACATGGGGGATCATGTAACTCGCCTTGATCGTTGGG |
| | AACCGGAGCTGAATGAAGCCATACCAAACGACGAGCGTGAC |
| | ACCACGATGCCTGTAGCAATGGCAACAACGTTGCGCAAACTA |
| | TTAACTGGCGAACTACTTACTCTAGCTTCCCGGCAACAATTAA |
| | TAGACTGGATGGAGGCGGATAAAGTTGCAGGACCACTTCTGC |
| | GCTCGGCCCTTCCGGCTGGCTGGTTTATTGCTGATAAATCTGG |
| | AGCCGGTGAGCGTGGAAGCCGCGGTATCATTGCAGCACTGGG |
| | GCCAGATGGTAAGCCCTCCCGTATCGTAGTTATCTACACGACG |
| | GGGAGTCAGGCAACTATGGATGAACGAAATAGACAGATCGCT |
| | GAGATAGGTGCCTCACTGATTAAGCATTGGTAACTGTCAGAC |
| | CAAGTTTACTCATATATACTTTAGATTGATTTAAAACTTCATTT |
| | TTAATTTAAAAGGATCTAGGTGAAGATCCTTTTTGATAATCTC |
| | ATGACCAAAATCCCTTAACGTGAGTTTTCGTTCCACTGAGCGT |
| | CAGACCCCGTAGAAAAGATCAAAGGATCTTCTTGAGATCCTT |
| | TTTTTCTGCGCGTAATCTGCTGCTTGCAAACAAAAAAACCACC |
| | GCTACCAGCGGTGGTTTGTTTGCCGGATCAAGAGCTACCAACT |
| | CTTTTTCCGAAGGTAACTGGCTTCAGCAGAGCGCAGATACCA |
| | AATACTGTCCTTCTAGTGTAGCCGTAGTTAGGCCACCACTTCA |
| | AGAACTCTGTAGCACCGCCTACATACCTCGCTCTGCTAATCCT |
| | GTTACCAGTGGCTGCTGCCAGTGGCGATAAGTCGTGTCTTACC |
| | GGGTTGGACTCAAGACGATAGTTACCGGATAAGGCGCAGCGG |
| | TCGGGCTGAACGGGGGGTTCGTGCACACAGCCCAGCTTGGAG |
| | CGAACGACCTACACCGAACTGAGATACCTACAGCGTGAGCTA |
| | TGAGAAAGCGCCACGCTTCCCGAAGGGAGAAAGGCGGACAG |
| | GTATCCGGTAAGCGGCAGGGTCGGAACAGGAGAGCGCACGA |
| | GGGAGCTTCCAGGGGGAAACGCCTGGTATCTTTATAGTCCTGT |
| | CGGGTTTCGCCACCTCTGACTTGAGCGTCGATTTTTGTGATGC |
| | TCGTCAGGGGGGCGGAGCCTATGGAAAAACGCCAGCAACGC |
| | GGCCTTTTTACGGTTCCTGGCCTTTTGCTGGCCTTTTGCTCACA |
| | TGT (SEQ ID NO: 34) |

Construction of targeting vector to knock-in mCherry in order to replace the targeted gene DAZL for sterility

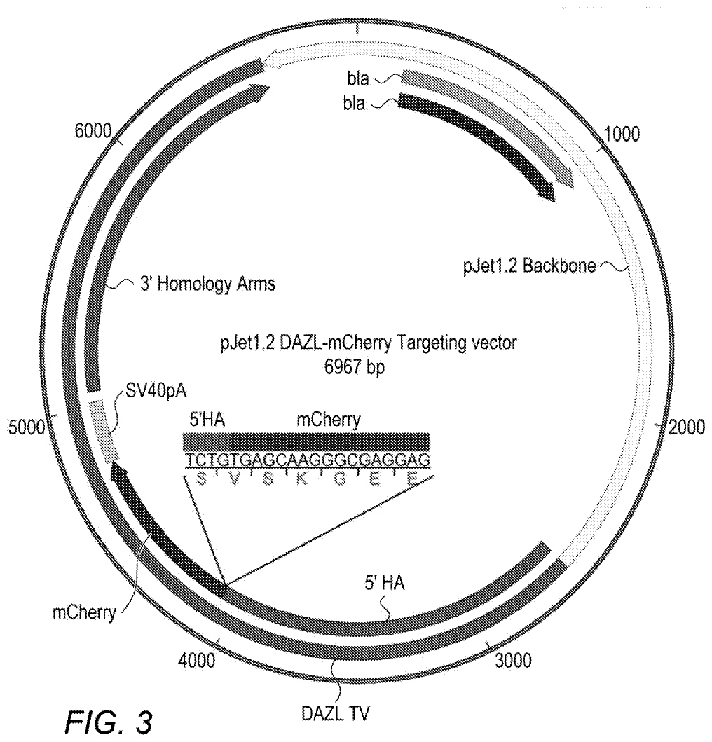

To generate the targeting vector (TV) for homologous recombination the DAZL-TV was designed and constructed (FIG. 1B and FIG. 3). The vector backbone plasmid for cloning is the pJet1.2 (THERMO FISHER SCIENTIFIC™) (FIG. 3). The insert contained the 5′ homology arm (5′ HA) followed by the coding sequence of mCherry, followed by polyadenylation site, followed by the 3′ homology arm (3′ HA). The 5′ HA consists of the 3′ end of the DAZL first intron and the four initial 5′ nucleotides of the second exon (TCTG). The 5′ HA sequence (SEQ ID NO: 11) is shown in TABLE 5.

Downstream of the 5′ HA sequence is the in-frame coding sequence of mCherry (mCherry CDS), excluding the first codon (ATG) of the initial mCherry methionine. This modified mCherry CDS sequence (SEQ ID NO: 12) is shown in TABLE 5.

Downstream of the modified mCherry CDS is the polyadenylation site (SV40). The polyadenylation site sequence (SEQ ID NO: 13) is shown in TABLE 5.

The 3′ HA consists of the 3′ end of exon 3, intron 3, exon 4, and the 5′ end of intron 4. The 3′ HA sequence (SEQ ID NO: 14) is shown in TABLE 5.

The genomic gap between the 5′ HA and the 3′ HA consists of most of exon 2, intron 2, and most of exon 3. The sequence (SEQ ID NO: 15) of the genomic gap between the 5′ HA and the 3′ HA is shown in TABLE 5 (see also FIG. 3), which in the reference genome GRCg6a, is located on Ch2: 34438202-34437740.

TABLE 5
Sequences used in the construction of the targeting vector
(TV) and targeting vector pJet1.2 DAZL-mCherry TV plasmid.

| Fragment | Sequence |
|---|---|
| 5′ HA | GCAGAGGAGGTGTAAGAGAAGAGATGGAGCAAAC |
| | TGTCTCTGCTTTTTAGGGGTAGTCTGGTGTTAATTTG |
| | GTTCACTCATGTTGAAATCATACACGTGGAAAGCTG |
| | AGTTCTAGTTCTGGCGTTACTTAATTGCTGGTTGAA |
| | TCAGGCCACATGGACAGTTTGGTTCTTGTAATTTTC |
| | TTAGTCCCTTACCGGATGTCCAATTTGAATGAACAT |
| | TGATCACTTGCAGGTGCAGTTTAAATAACTCTGTGT |
| | TAAACTTCTAATTTGTTACAATCACGAGAGCCCATT |
| | TTTCAATGTAAATTATTCTTAGGTTTCAAAGTATCA |
| | GTAACCTCAACTAAATCAAAAGATCTGCCTATCTGA |
| | AATAGGGATAATGCTACACCAGGTGAGCTGCAAGG |
| | AAAAGGTTATTAATGTTTTGAGATGTCTTAATACAG |
| | ACAAATGAGCACAAATAAGGTGGTCAAAGTAGTTG |
| | TTTTTTTTACAAGCCAGATAAAGAATGACATGTACA |
| | TAGAACCATTCATTCAGTTGAGAAGATGTGGAACC |
| | AAAGTTTCATCCATCTGAGGTGTATTTCAGTTCTTG |
| | CAAATATCTTTGTGTAATGTTTGAAGTGTGTTTTAG |
| | AGTATGGAACACGTCTTGGTGTCATCAGCAACAAG |
| | AAATGGAATTGTGTGGTCTCTGTGAACAAATGATTC |
| | CCTAAATAAACAGTAATCCAGAATCCACTTTCCTCT |
| | GACCTGAACTGAGTGAGAAACTTTGAGGCTGTGAG |
| | TTACGTTCAAGTTTAAAGGGTGCACGTGGAATGTGG |
| | GTGTGCGAAGCACATCACCGCTGTAGTTATTCCATT |
| | ACACCATGTAGATATGTGCAGTGCACTCTTAAGATC |
| | CTGCTTCGGTGTGTGCCACTCAGTGACAAGATCAGT |
| | CGTTCATATTTCTCTTGTAGTTAAATTGACTAAAAC |
| | TTTTTTTCTGAGTGCACATAAAGTGAATATTCTACC |
| | AAGACGGTGTCATCTACCTTACAGCTAAATTGTAGT |
| | ATAACTGTACCATTGTTCCAAGGAATTTCATAAGCT |
| | TTACTCACTTCTTGAGCATTACAGGCTTTTGATCAG |
| | GAAAATGGAGTTCATTCATTGGATAATCAATTCACA |
| | GTGTAGAACTTAAGAATTTCTTGCTTGCATCTAAAA |
| | GGAATTGTGTAAAAATTTGCGATGAATAATTTCGGG |
| | GTTCTCATTGTAAGTTGGGTAGTAGCAACAATGGTT |
| | GTGAAGCTTCCAGTCAGGCAAGGCTGCTTAGTGTA |
| | GCCTAGCTTGAGTCTTGATCTCAAAGAAGAGTAGGT |
| | TCAAACTTGTCCATTTGAGTGTTCCATTTGCAGTAA |
| | GTACTTCCTTTAAGTAATTGGAAAGATGCTATTGAT |
| | ACTTACGGTCTCAAACTGTATCAATAGGAAGATGG |
| | AGGCCTACCTAGTAGTGTATTACAATGTGACTGAAA |
| | TATTTCTGTTTAACCTTTTCAGTCTG (SEQ ID NO: 11) |
| mCherry CDS | TGAGCAAGGGCGAGGAGGATAACATGGCCATCATC |
| | AAGGAGTTCATGCGCTTCAAGGTGCACATGGAGGG |
| | CTCCGTGAACGGCCACGAGTTCGAGATCGAGGGCG |
| | AGGGCGAGGGCCGCCCCTACGAGGGCACCCAGACC |
| | GCCAAGCTGAAGGTGACCAAGGGTGGCCCCCTGCC |
| | CTTCGCCTGGGACATCCTGTCCCCTCAGTTCATGTA |
| | CGGCTCCAAGGCCTACGTGAAGCACCCCGCCGACA |
| | TCCCCGACTACTTGAAGCTGTCCTTCCCCGAGGGCT |
| | TCAAGTGGGAGCGCGTGATGAACTTCGAGGACGGC |
| | GGCGTGGTGACCGTGACCCAGGACTCCTCCCTGCA |
| | GGACGGCGAGTTCATCTACAAGGTGAAGCTGCGCG |
| | GCACCAACTTCCCCTCCGACGGCCCCGTAATGCAGA |
| | AGAAGACCATGGGCTGGGAGGCCTCCTCCGAGCGG |
| | ATGTACCCCGAGGACGGCGCCCTGAAGGGCGAGAT |
| | CAAGCAGAGGCTGAAGCTGAAGGACGGCGGCCACT |
| | ACGACGCTGAGGTCAAGACCACCTACAAGGCCAAG |
| | AAGCCCGTGCAGCTGCCCGGCGCCTACAACGTCAA |
| | CATCAAGTTGGACATCACCTCCCACAACGAGGACT |
| | ACACCATCGTGGAACAGTACGAACGCGCCGAGGGC |
| | CGCCACTCCACCGGCGGCATGGACGAGCTGTACAA |
| | GTAA (SEQ ID NO: 12) |
| Polyadenylation | CGCGGGCCCGGGATCCACCGGATCTAGATAACTGA |
| Site | TCATAATCAGCCATACCACATTTGTAGAGGTTTTAC |
| | TTGCTTTAAAAAACCTCCCACACCTCCCCCTGAACC |
| | TGAAACATAAAATGAATGCAATTGTTGTTGTTAACT |
| | TGTTTATTGCAGCTTATAATGGTTACAAATAAAGCA |
| | ATAGCATCACAAATTTCACAAATAAAGCATTTTTTT |
| | CACTGCATTCTAGTTGTGGTTTGTCCAAACT (SEQ ID |
| | NO: 13) |
| 3′ HA | ACTGACAGAACTGGTGTTTCCAAAGGGTGAGCAGA |
| | ATGTCATTAGTTACTGCTTTTGTAGATGTAATTCTA |
| | ACATAAATGATGTCTGTTGTTAAGTAGTTGGTCACT |
| | TACCATGCTTAAGCCTTTCAAACTGGGGTGAATTAA |
| | AGTGAAACATGTAAGATCATATAGATTTAAGATCA |
| | GTCAAGTTTTACAATTGAGAACTGGACAGATTTTAT |
| | GGTGTACCTGTTCAGGGAAAATAGTGTTAATGTCAC |
| | TCAACCAGTGGGAGCAAAGCATAAAACGTAGTGGA |
| | TGCTTGTGGGGACTGTTTTACAGGCTGAAATTTTGA |
| | CTTTCTGATGGCCATAGCAATTAAGCAGCCATCAGT |
| | GTAGTACCACTAATGTAATTGAGACAGGGAGTAGA |
| | CTTTCATTGGGGCAGTTGGACTGCAGTCTTTTGTTG |
| | CTCAGGGGTAAGTTAGAGGCAATCAAACTGTTTCA |
| | GGTGGTGAGTGAAACTTAAGGGATGGTAGAAAATT |
| | AGAGACATTCCCATTGGATATGTAGAAAGTACTCTG |
| | ATCTGTAGTGAAGAACTTAAGTGAAGATGCCTAGG |
| | ACTCTGCCCAGTTGAGTTCAGAGGAAGCTCTCCCAG |
| | CTTTGAAATTAGACTTGCTTTGCGAGGAAGACTTCA |
| | CCTCTAAAGATGCACCAATTGTTTTCTCTGAGCAGG |
| | TTCCAAAAAGTAGCATTTTTTTTTTAATAGACACAT |
| | ATAGTAATGAGCTGAAAATACTGAGCTTAATGTCTC |
| | TTGCCTGGTCTTTGTGGTGAATTCTAATGTGTGATT |
| | AGCAAGCATATGTTCTGATTATTGATAAATTGCTGT |
| | ATGTCAATCAGTGGAATACTCTACTGCAGTTCTGAG |
| | AATTGTCTCCAATATTAAGGCTTAAATAAACAAGA |
| | GGTAGTGAGATAAATTGAAAACCTCTTTTGGGATCG |
| | CTTCCTCCAATAGTGTAATTATTCCTGTAGTTCCTCC |
| | TTTCATTCAAACCTCTGCAGGAAGTACAGAATTTAG |
| | TACATACTAATTGAAGGAGCTTTTGGCTTTCTGATG |
| | CTACTAATATTAACAGTAGTACTCACTTGAGTAATT |
| | TAAATGAGAGAATATTGAATGTGGCATTTAATTCCT |
| | TTCATTTGGCCCAGTGTGCTGTCAGTCAGCAGCAAA |
| | TGTACTTTCATGCTGAATTATATATTAATGTCCTGTT |
| | AATATCAGTTAATGTTCTTTTTACTGTTTTAGTTTTT |
| | TTTAAAAAAAAAACTAACAGCTGTCAAAAAATGAA |
| | AATGTAGTATTTGAATAATATTTTTTTTCTTTTCAGG |
| | TATGGATTTGTTTCATTCCTGGACAATGTGGATGTT |
| | CAAAAGATAGTAGAAGTAAGCTCTTTATGTCTTAAG |
| | TTGTCAGAAGAACCTTCTGTATGAAGGTTGTAGGTG |
| | TGGTTAGGGGATACCAGTCCCAACTGAGAAAATAA |
| | AAAAGACTAGAAGTGCCCCAAAGTAAACTTGCTTA |
| | AATATTGTTGTGATTTAACCCAGCAGATTGTGAAGT |
| | ACCATGTAGTATTTTCCTCACTGCACTCC (SEQ ID |
| | NO: 14) |
| Genomic Gap | CAAATGCGGAAGCCCAGTGTGGAACTATCTCAGAG |
| Between | GATAATACCCATTCGTCAACAACCTGCCAAGGATAT |
| 5′ HA and 3′ HA | GTTTTACCAGAAGGAAAAATCATGCCAAATACAGT |
| | CTTTGTTGGTGGAATTGATATAAGGGTATTTATGTA |
| | CTTTCAATGGTTTTAAACTACATATGACACGCTGTA |
| | GTGGGAAAGAAATAAGAATTTTAACTTCTGGAGGG |
| | CTTTTTTTTAATTGGGTCTTTACTGATCTTGAAATAA |
| | TGCATTATGGTAAGAGAACTTTGAAACAAACAAAA |
| | GAGATTTTCCTGGAATATTAGGAGATGTGTTTAAAA |
| | ATGGTACTTGTTGCTTTAAAACAATTGTAACCGTTT |
| | ACTGTGTCTGTGAAGTAGTTCAAGACTTGGTTTCTT |
| | TTAGATGAATGAAGCAGAAATTCGGAGTTACTTTG |
| | AACAATATGGTACTGTGAAGGAGGTGAAAATAATC |
| | (SEQ ID NO: 15) |
| pJet1.2 DAZL- | GGCCTCGTGATACGCCTATTTTTATAGGTTAATGTC |
| mCherry TV | ATGATAATAATGGTTTCTTAGACGTCAGGTGGCACT |
| plasmid | TTTCGGGGAAATGTGCGCGGAACCCCTATTTGTTTA |
| | TTTTTCTAAATACATTCAAATATGTATCCGCTCATG |
| | AGACAATAACCCTGATAAATGCTTCAATAATATTGA |
| | AAAAGGAAGAGTATGAGTATTCAACATTTCCGTGT |
| | CGCCCTTATTCCCTTTTTTGCGGCATTTTGCCTTCCT |
| | GTTTTTGCTCACCCAGAAACGCTGGTGAAAGTAAA |
| | AGATGCTGAAGATCAGTTGGGTGCACGAGTGGGTT |
| | ACATCGAACTGGATCTCAACAGCGGTAAGATCCTT |
| | GAGAGTTTTCGCCCCGAAGAACGTTTTCCAATGATG |
| | AGCACTTTTAAAGTTCTGCTATGTGGCGCGGTATTA |
| | TCCCGTATTGACGCCGGGCAAGAGCAACTCGGTCG |
| | CCGCATACACTATTCTCAGAATGACTTGGTTGAGTA |
| | CTCACCAGTCACAGAAAAGCATCTTACGGATGGCA |
| | TGACAGTAAGAGAATTATGCAGTGCTGCCATAACC |
| | ATGAGTGATAACACTGCGGCCAACTTACTTCTGACA |
| | ACGATCGGAGGACCGAAGGAGCTAACCGCTTTTTT |
| | GCACAACATGGGGGATCATGTAACTCGCCTTGATC |
| | GTTGGGAACCGGAGCTGAATGAAGCCATACCAAAC |
| | GACGAGCGTGACACCACGATGCCTGTAGCAATGGC |
| | AACAACGTTGCGCAAACTATTAACTGGCGAACTAC |
| | TTACTCTAGCTTCCCGGCAACAATTAATAGACTGGA |
| | TGGAGGCGGATAAAGTTGCAGGACCACTTCTGCGC |
| | TCGGCCCTTCCGGCTGGCTGGTTTATTGCTGATAAA |
| | TCTGGAGCCGGTGAGCGTGGGTCTCGCGGTATCATT |
| | GCAGCACTGGGGCCAGATGGTAAGCCCTCCCGTAT |
| | CGTAGTTATCTACACGACGGGGAGTCAGGCAACTA |
| | TGGATGAACGAAATAGACAGATCGCTGAGATAGGT |
| | GCCTCACTGATTAAGCATTGGTAACTGTCAGACCAA |
| | GTTTACTCATATATACTTTAGATTGATTTAAAACTTC |
| | ATTTTTAATTTAAAAGGATCTAGGTGAAGATCCTTT |
| | TTGATAATCTCATGACCAAAATCCCTTAACGTGAGT |
| | TTTCGTTCCACTGAGCGTCAGACCCCGTAGAAAAGA |
| | TCAAAGGATCTTCTTGAGATCCTTTTTTTCTGCGCGT |
| | AATCTGCTGCTTGCAAACAAAAAAACCACCGCTAC |
| | CAGCGGTGGTTTGTTTGCCGGATCAAGAGCTACCAA |
| | CTCTTTTTCCGAAGGTAACTGGCTTCAGCAGAGCGC |
| | AGATACCAAATACTGTCCTTCTAGTGTAGCCGTAGT |
| | TAGGCCACCACTTCAAGAACTCTGTAGCACCGCCTA |
| | CATACCTCGCTCTGCTAATCCTGTTACCAGTGGCTG |
| | CTGCCAGTGGCGATAAGTCGTGTCTTACCGGGTTGG |
| | ACTCAAGACGATAGTTACCGGATAAGGCGCAGCGG |
| | TCGGGCTGAACGGGGGGTTCGTGCACACAGCCCAG |
| | CTTGGAGCGAACGACCTACACCGAACTGAGATACC |
| | TACAGCGTGAGCTATGAGAAAGCGCCACGCTTCCC |
| | GAAGGGAGAAAGGCGGACAGGTATCCGGTAAGCG |
| | GCAGGGTCGGAACAGGAGAGCGCACGAGGGAGCTT |
| | CCAGGGGGAAACGCCTGGTATCTTTATAGTCCTGTC |
| | GGGTTTCGCCACCTCTGACTTGAGCGTCGATTTTTG |
| | TGATGCTCGTCAGGGGGGCGGAGCCTATGGAAAAA |
| | CGCCAGCAACGCGGCCTTTTTACGGTTCCTGGCCTT |
| | TTGCTGGCCTTTTGCTCACATGTTCTTTCCTGCGTTA |
| | TCCCCTGATTCTGTGGATAACCGTATTACCGCCTTT |
| | GAGTGAGCTGATACCGCTCGCCGCAGCCGAACGAC |
| | CGAGCGCAGCGAGTCAGTGAGCGAGGAAGCGGAA |
| | GAGCGCCCAATACGCAAACCGCCTCTCCCCGCGCG |
| | TTGGCCGATTCATTAATGCAGCTGGCACGACAGGTT |
| | TCCCGACTGGAAAGCAATTGGCAGTGAGCGCAACG |
| | CAATTAATGTGAGTTAGCTCACTCATTAGGCACCCC |
| | AGGCTTTACACTTTATGCTTCCGGCTCGTATAATGT |
| | GTGGAATTGTGAGCGGATAACAATTTCACACAGGA |
| | GGTTTAAACTTTAAACATGTCAAAAGAGACGTCTTT |
| | TGTTAAGAATGCTGAGGAACTTGCAAAGCAAAAAA |
| | TGGATGCTATTAACCCTGAACTTTCTTCAAAATTTA |
| | AATTTTTAATAAAATTCCTGTCTCAGTTTCCTGAAG |
| | CTTGCTCTAAACCTCGTTCAAAAAAAATGCAGAATA |
| | AAGTTGGTCAAGAGGAACATATTGAATATTTAGCTC |
| | GTAGTTTTCATGAGAGTCGATTGCCAAGAAAACCC |
| | ACGCCACCTACAACGGTTCCTGATGAGGTGGTTAGC |
| | ATAGTTCTTAATATAAGTTTTAATATACAGCCTGAA |
| | AATCTTGAGAGAATAAAAGAAGAACATCGATTTTC |
| | CATGGCAGCTGAGAATATTGTAGGAGATCTTCTAG |
| | AAAGATGCAGAGGAGGTGTAAGAGAAGAGATGGA |
| | GCAAACTGTCTCTGCTTTTTAGGGGTAGTCTGGTGT |
| | TAATTTGGTTCACTCATGTTGAAATCATACACGTGG |
| | AAAGCTGAGTTCTAGTTCTGGCGTTACTTAATTGCT |
| | GGTTGAATCAGGCCACATGGACAGTTTGGTTCTTGT |
| | AATTTTCTTAGTCCCTTACCGGATGTCCAATTTGAA |
| | TGAACATTGATCACTTGCAGGTGCAGTTTAAATAAC |
| | TCTGTGTTAAACTTCTAATTTGTTACAATCACGAGA |
| | GCCCATTTTTCAATGTAAATTATTCTTAGGTTTCAA |
| | AGTATCAGTAACCTCAACTAAATCAAAAGATCTGC |
| | CTATCTGAAATAGGGATAATGCTACACCAGGTGAG |
| | CTGCAAGGAAAAGGTTATTAATGTTTTGAGATGTCT |
| | TAATACAGACAAATGAGCACAAATAAGGTGGTCAA |
| | AGTAGTTGTTTTTTTTACAAGCCAGATAAAGAATGA |
| | CATGTACATAGAACCATTCATTCAGTTGAGAAGATG |
| | TGGAACCAAAGTTTCATCCATCTGAGGTGTATTTCA |
| | GTTCTTGCAAATATCTTTGTGTAATGTTTGAAGTGT |
| | GTTTTAGAGTATGGAACACGTCTTGGTGTCATCAGC |
| | AACAAGAAATGGAATTGTGTGGTCTCTGTGAACAA |
| | ATGATTCCCTAAATAAACAGTAATCCAGAATCCACT |
| | TTCCTCTGACCTGAACTGAGTGAGAAACTTTGAGGC |
| | TGTGAGTTACGTTCAAGTTTAAAGGGTGCACGTGGA |
| | ATGTGGGTGTGCGAAGCACATCACCGCTGTAGTTAT |
| | TCCATTACACCATGTAGATATGTGCAGTGCACTCTT |
| | AAGATCCTGCTTCGGTGTGTGCCACTCAGTGACAAG |
| | ATCAGTCGTTCATATTTCTCTTGTAGTTAAATTGACT |
| | AAAACTTTTTTTCTGAGTGCACATAAAGTGAATATT |
| | CTACCAAGACGGTGTCATCTACCTTACAGCTAAATT |
| | GTAGTATAACTGTACCATTGTTCCAAGGAATTTCAT |
| | AAGCTTTACTCACTTCTTGAGCATTACAGGCTTTTG |
| | ATCAGGAAAATGGAGTTCATTCATTGGATAATCAAT |
| | TCACAGTGTAGAACTTAAGAATTTCTTGCTTGCATC |
| | TAAAAGGAATTGTGTAAAAATTTGCGATGAATAAT |
| | TTCGGGGTTCTCATTGTAAGTTGGGTAGTAGCAACA |
| | ATGGTTGTGAAGCTTCCAGTCAGGCAAGGCTGCTTA |
| | GTGTAGCCTAGCTTGAGTCTTGATCTCAAAGAAGAG |
| | TAGGTTCAAACTTGTCCATTTGAGTGTTCCATTTGC |
| | AGTAAGTACTTCCTTTAAGTAATTGGAAAGATGCTA |
| | TTGATACTTACGGTCTCAAACTGTATCAATAGGAAG |
| | ATGGAGGCCTACCTAGTAGTGTATTACAATGTGACT |
| | GAAATATTTCTGTTTAACCTTTTCAGTCTGTGAGCA |
| | AGGGCGAGGAGGATAACATGGCCATCATCAAGGAG |
| | TTCATGCGCTTCAAGGTGCACATGGAGGGCTCCGTG |
| | AACGGCCACGAGTTCGAGATCGAGGGCGAGGGCGA |
| | GGGCCGCCCCTACGAGGGCACCCAGACCGCCAAGC |
| | TGAAGGTGACCAAGGGTGGCCCCCTGCCCTTCGCCT |
| | GGGACATCCTGTCCCCTCAGTTCATGTACGGCTCCA |
| | AGGCCTACGTGAAGCACCCCGCCGACATCCCCGAC |
| | TACTTGAAGCTGTCCTTCCCCGAGGGCTTCAAGTGG |
| | GAGCGCGTGATGAACTTCGAGGACGGCGGCGTGGT |
| | GACCGTGACCCAGGACTCCTCCCTGCAGGACGGCG |
| | AGTTCATCTACAAGGTGAAGCTGCGCGGCACCAAC |
| | TTCCCCTCCGACGGCCCCGTAATGCAGAAGAAGAC |
| | CATGGGCTGGGAGGCCTCCTCCGAGCGGATGTACC |
| | CCGAGGACGGCGCCCTGAAGGGCGAGATCAAGCAG |
| | AGGCTGAAGCTGAAGGACGGCGGCCACTACGACGC |
| | TGAGGTCAAGACCACCTACAAGGCCAAGAAGCCCG |
| | TGCAGCTGCCCGGCGCCTACAACGTCAACATCAAG |
| | TTGGACATCACCTCCCACAACGAGGACTACACCATC |
| | GTGGAACAGTACGAACGCGCCGAGGGCCGCCACTC |
| | CACCGGCGGCATGGACGAGCTGTACAAGTAAGGGT |
| | ACCGCGGGCCCGGGATCCACCGGATCTAGATAACT |
| | GATCATAATCAGCCATACCACATTTGTAGAGGTTTT |
| | ACTTGCTTTAAAAAACCTCCCACACCTCCCCCTGAA |
| | CCTGAAACATAAAATGAATGCAATTGTTGTTGTTAA |
| | CTTGTTTATTGCAGCTTATAATGGTTACAAATAAAG |
| | CAATAGCATCACAAATTTCACAAATAAAGCATTTTT |
| | TTCACTGCATTCTAGTTGTGGTTTGTCCAAACTCATC |
| | AATGTATCTTAAGATCTACTAGTAGATCCATGCATA |
| | TCACTGACAGAACTGGTGTTTCCAAAGGGTGAGCA |
| | GAATGTCATTAGTTACTGCTTTTGTAGATGTAATTC |
| | TAACATAAATGATGTCTGTTGTTAAGTAGTTGGTCA |
| | CTTACCATGCTTAAGCCTTTCAAACTGGGGTGAATT |
| | AAAGTGAAACATGTAAGATCATATAGATTTAAGAT |
| | CAGTCAAGTTTTACAATTGAGAACTGGACAGATTTT |
| | ATGGTGTACCTGTTCAGGGAAAATAGTGTTAATGTC |
| | ACTCAACCAGTGGGAGCAAAGCATAAAACGTAGTG |
| | GATGCTTGTGGGGACTGTTTTACAGGCTGAAATTTT |
| | GACTTTCTGATGGCCATAGCAATTAAGCAGCCATCA |
| | GTGTAGTACCACTAATGTAATTGAGACAGGGAGTA |
| | GACTTTCATTGGGGCAGTTGGACTGCAGTCTTTTGT |
| | TGCTCAGGGGTAAGTTAGAGGCAATCAAACTGTTTC |
| | AGGTGGTGAGTGAAACTTAAGGGATGGTAGAAAAT |
| | TAGAGACATTCCCATTGGATATGTAGAAAGTACTCT |
| | GATCTGTAGTGAAGAACTTAAGTGAAGATGCCTAG |
| | GACTCTGCCCAGTTGAGTTCAGAGGAAGCTCTCCCA |
| | GCTTTGAAATTAGACTTGCTTTGCGAGGAAGACTTC |
| | ACCTCTAAAGATGCACCAATTGTTTTCTCTGAGCAG |
| | GTTCCAAAAAGTAGCATTTTTTTTTTAATAGACACA |
| | TATAGTAATGAGCTGAAAATACTGAGCTTAATGTCT |
| | CTTGCCTGGTCTTTGTGGTGAATTCTAATGTGTGATT |
| | AGCAAGCATATGTTCTGATTATTGATAAATTGCTGT |
| | ATGTCAATCAGTGGAATACTCTACTGCAGTTCTGAG |
| | AATTGTCTCCAATATTAAGGCTTAAATAAACAAGA |
| | GGTAGTGAGATAAATTGAAAACCTCTTTTGGGATCG |
| | CTTCCTCCAATAGTGTAATTATTCCTGTAGTTCCTCC |
| | TTTCATTCAAACCTCTGCAGGAAGTACAGAATTTAG |
| | TACATACTAATTGAAGGAGCTTTTGGCTTTCTGATG |
| | CTACTAATATTAACAGTAGTACTCACTTGAGTAATT |
| | TAAATGAGAGAATATTGAATGTGGCATTTAATTCCT |
| | TTCATTTGGCCCAGTGTGCTGTCAGTCAGCAGCAAA |
| | TGTACTTTCATGCTGAATTATATATTAATGTCCTGTT |
| | AATATCAGTTAATGTTCTTTTTACTGTTTTAGTTTTT |
| | TTTAAAAAAAAAACTAACAGCTGTCAAAAAATGAA |
| | AATGTAGTATTTGAATAATATTTTTTTTCTTTTCAGG |
| | TATGGATTTGTTTCATTCCTGGACAATGTGGATGTT |
| | CAAAAGATAGTAGAAGTAAGCTCTTTATGTCTTAAG |
| | TTGTCAGAAGAACCTTCTGTATGAAGGTTGTAGGTG |
| | TGGTTAGGGGATACCAGTCCCAACTGAGAAAATAA |
| | AAAAGACTAGAAGTGCCCCAAAGTAAACTTGCTTA |
| | AATATTGTTGTGATTTAACCCAGCAGATTGTGAAGT |
| | ACCATGTAGTATTTTCCTCACTGCACTCCATCTTGCT |
| | GAAAAACTCGAGCCATCCGGAAGATCTGGCGGCCG |
| | CTCTCCCTATAGTGAGTCGTATTACGCCGGATGGAT |
| | ATGGTGTTCAGGCACAAGTGTTAAAGCAGTTGATTT |
| | TATTCACTATGATGAAAAAAACAATGAATGGAACC |
| | TGCTCCAAGTTAAAAATAGAGATAATACCGAAAAC |
| | TCATCGAGTAGTAAGATTAGAGATAATACAACAAT |
| | AAAAAAATGGTTTAGAACTTACTCACAGCGTGATG |
| | CTACTAATTGGGACAATTTTCCAGATGAAGTATCAT |
| | CTAAGAATTTAAATGAAGAAGACTTCAGAGCTTTTG |
| | TTAAAAATTATTTGGCAAAAATAATATAATTCGGCT |
| | GCAGGGGC (SEQ ID NO: 37) |

FIG. 3 shows the targeting vector (TV) plasmid map with mCherry. In FIG. 3, the locations of the 5′ HA and mCherry sequences are indicated, and a portion of the coding region of the 5′ HA-mCherry sequence is shown (inset). The DNA sequence is shown in black (SEQ ID NO: 35), while the corresponding encoded protein is shown in red (SEQ ID NO: 36). The overall sequence of the pJet1.2 DAZL-mCherry TV plasmid is SEQ ID NO: 37, as shown in TABLE 5. Notably, as shown in FIG. 1B, all the sgRNA CRISPR sites (CRISPR 1-3; see TABLE 3) are located in the region of the genomic gap between 5′ HA and 3′ HA. Thus, the TV itself does not contain the recognition sites, ensuring that the TV will not be cleaved by the CRISPR-Cas9 system upon co-transfection of both elements. Moreover, the mCherry sequence does not contain a promoter. Therefore, the TV, when transfected to PGCs, does not result in red fluorescence encoded by the mCherry sequence (SEQ ID NO: 24). However, upon correct homologous recombination, the mCherry coding sequence is designed to integrate in-frame, into the DAZL locus, which is expressed and active in PGCs. Thus, following transfection of the TV, red fluorescence expression of mCherry serves as a biological confirmation of the correct integration. Here, the expression of mCherry depends on the DAZL promoter. Part of the endogenous DAZL promoter is located on the 5′HA on the targeting vector. This part is insufficient to drive mCherry expression. Thus, when transfected to PGCs, the TV does not express mCherry. Only upon homologous recombination (HR) integration, does the mCherry become active.

The sequence of the genomic regions of the 5′ HA was taken from the chromosome: GRCg6a 2:34438203-34439663 (see SEQ ID NO: 11).

Transfection of PGCs and obtaining purified, genetically-modified colonies Plasmid transfection of PGCs was done using lipofection or electroporation. For lipofection, LIPOFECTAMINE™ 2000 (INVITROGEN™) was used according to the manufacturer's protocol. 3-5×105 cells were seeded in 96 well plate in Avian Knockout Dulbecco's Modified Eagle's Medium (AKODMEM™) containing non-essential amino acids (NEAA), pyruvate, vitamins, CaCl₂ and growth factors (activinA, human fibroblast growth factor [hFGF], and ovatransferin). (AKODMEM™ consists of Dulbecco's Modified Eagle's Medium (DMEM; GIBCO™) calcium free medium diluted with water to 250 milliosmolar (mosmol)/L, containing 12.0 mM glucose, 2.0 mM GLUTAMAX™ (GIBCO™), 1.2 mM pyruvate (GIBCO™), 1×Minimum Essential Medium (MEM) vitamin (GIBCO™), 1×B-27 supplement (GIBCO™), 1×non-essential amino acid (NEAA) supplement (GIBCO™), 0.1 mM β-mercaptoethanol (2-mercaptoethanol; GIBCO™), 1×nucleosides (BIOLOGICAL INDUSTRIES™), 0.2% ovalbumin (SIGMA™), 0.1 mg/ml sodium heparin (SIGMA™), CaCl₂ 0.15 mM (SIGMA™), 1×MEM vitamin (GIBCO™), 1× Pen/Strep (BIOLOGICAL INDUSTRIES™), 0.2% chicken serum (SIGMA™) in avian DMEM. The following growth factors were added before use: human Activin A, 25 ng/mL (PEPROTECH™); human FGF2 4 ng/mL (R&D BIOSYSTEMS), ovotransferin (5 μg/ml) (SIGMA™).)

100 ng of plasmid, and 0.25 microliters (μ1) of LIPOFECTAMINE′ 2000 (INVITROGEN™) were diluted separately in 20 μl of OPTI-MEM™ (GIBCO™) mix together, incubated for 20 minutes and pipetted on the cells. OPTI-MEM™ is a modification of Eagle's Minimum Essential Medium (MEM), buffered with 4-(2-hydroxyethyl)-1-piperazineethanesulfonic acid (HEPES) and sodium bicarbonate, and supplemented with hypoxanthine, thymidine, sodium pyruvate, L-glutamine, trace elements, and growth factors. OPTI-MEM™ is a modification of Eagle's Minimum Essential Medium, buffered with HEPES and sodium bicarbonate, and supplemented with hypoxanthine, thymidine, sodium pyruvate, L-glutamine, trace elements, and growth factors. For electroporation, 5×10⁵ or 1.5×10⁶ cells were washed in AKODMEM™ (see above), then transferred to buffer “R” (NEON buffer, INVITROGEN′) containing plasmids at concentration of 1 μg/μ1, and electroporated by 3 pulses of 1000V, 15 milliseconds (ms) duration each, using NEON electroporator (INVITROGEN™). Thereafter, the cells were seeded immediately in 96 or 48 well plate, respectively, in antibiotics-free PGCs medium. Medium was changed after 1-3 hours, and transfected cells were allowed to recover for 4-10 days. Transfected cells were individually isolated by fluorescence-activated cell sorting (FACS) sorting. For FACS sorting, gentle cells pipetting was done and cells were sorted in PGCs culture medium. Positive mCherry cells were sorted with FACSMELODY™ sorter (BD™, USA) to 96 wells plate, containing AKODMEM™.

Individual cells were grown to form pure colonies. From these colonies, total genomic DNA was extracted for analysis. Primers were designed, as shown in Table 6.

TABLE 6
Primers for analysis of genomic integration sites.

| Primer | | Primer Sequence |
|---|---|---|
| name | Target | (SEQ ID NO:) |
| P1 | 5′ integration site | TGATAGCACAGTAAGCTGATTC |
| | (FWD) | (SEQ ID NO: 16) |
| P2 | 5′ integration site | CGTACATGAACTGAGGGGAC |
| | (REV) | (SEQ ID NO: 17) |
| P3 | 3′ integration site | TTGTTTATTGCAGCTTATAATG |
| | (FWD) | (SEQ ID NO: 18) |
| P4 | 3′ integration site | ACCCCGTTACCCATTTTTCC |
| | (REV) | (SEQ ID NO: 19) |
| P5 | Flanking primer | CCTTTTCAGTCTGCAAATGC |
| | (FWD) | (SEQ ID NO: 20) |
| P6 | Flanking primer | ATTTTCCCTGAACAGATACACC |
| | (REV) | (SEQ ID NO: 21) |

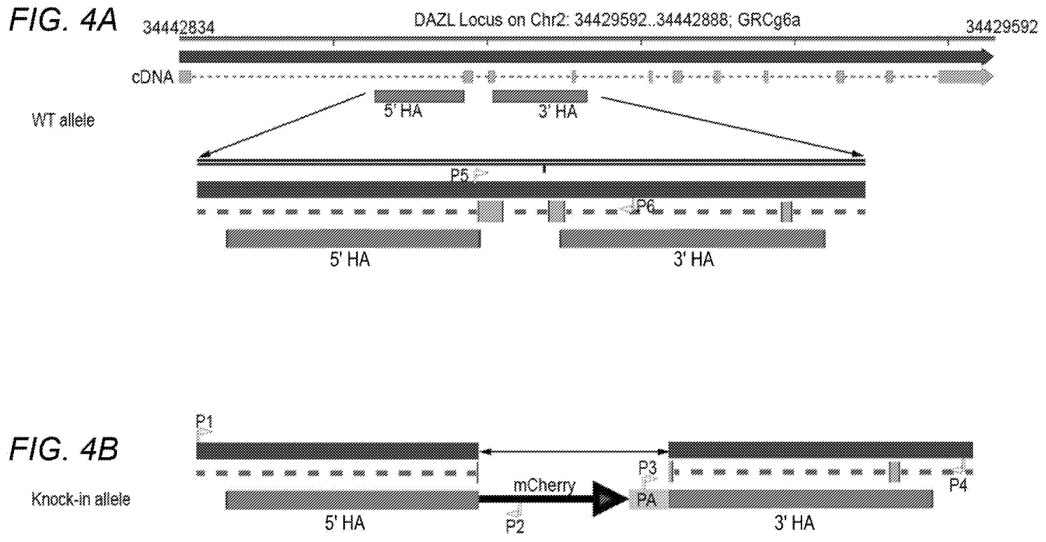

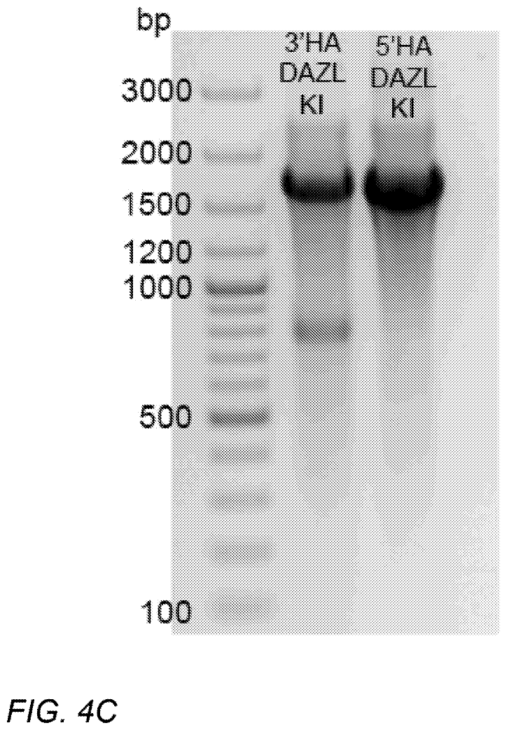

To confirm correct integration of the TV (FIGS. 4A-4B), two sets of primers, for the 5′ and the 3′ integration sites were designed (FIG. 4B). For the 5′ integration site, the forward (FWD) primer (P1) is located upstream and outside the 5′HA, and the reverse (REV) primer (P2) is located in the mCherry gene. For the 3′ integration site, the FWD primer (P3) is located in the mCherry region, and the REV primer (P4) is located downstream and outside the 3′ HA region (FIG. 4B, showing the knock-in allele with the primer locations [P1-P4]). The predicted PCR product sizes of these reaction is 1794 bp and 1803 bp, respectively (FIG. 4C).

To validate the integrity of the WT allele, a PCR reaction was designed to amplify the region flanking the CRISPR sites (FIG. 4A). The sequences of the FWD primer (P5) and the REV primer (P6) used for this reaction are shown in Table 6. respectively (as denoted in FIG. 4A). This product was sequenced to confirm the integrity of the WT allele, confirming the sequence in Table 7.

TABLE 7
Nucleotide Sequence of Region Flanking
the CRISPR Sites.

| PCR Product | Sequence |
|---|---|
| Flanking | CCTTTTCAGTCTGCAAATGCGGAAGCCCAGTGTGG |
| the CRISPR | AAGTATCTCAGAGGATAATACCCATTCGTCAACAA |
| Sites | CCTGCCAAGGATATGTTTTACCAGAAGGAAAAATC |
| | ATGCCAAATACAGTCTTTGTTGGTGGAATTGATAT |
| | AAGGGTATTTATGTACTTTCAATGGTTTTAAACTAC |
| | ATATGACACGCTGTAGTGGGAAAGAAATAAGAAT |
| | TTTAACTTCTGGAGGGCTTTTTTTTAATTGGGTCTT |
| | TACCGATCTTGAAATAATGCATTATGGTAAGAGAA |
| | CTTTGAAACAAACAAGAGAGATTTTCCTGGAATAT |
| | TAGGAGATGTGTTTAAAAATGGTACTTGTTGCTTTA |
| | AAACAATTGTAACCGTTTACTGTGTCTGTGAAGTA |
| | GTTCAAGACTTGGTTTCTTTTAGATGAATGAAGCA |
| | GAAATTCGGAGTTACTTTGAACAATATGGTACTGT |
| | GAAGGAGGTGAAAATAATCACTGACAGAACTGGT |
| | GTTTCCAAAGGGTGAGCAGAATGTCATTAGTTACT |
| | GCTTTTGTAGATGTAATTCTAACATAAATGATGTCT |
| | GTTGTTAAGTAGTTGGTCACTTACCATGCTTAAGCC |
| | TTTCAAATTGAGATGAATTAAAGTGAAACATGTAA |
| | AACTCGACTGATCTTAAATCTGTATAATCTTACAAT |
| | TGAGAACTGGACATATTTTATGGTGTATCTGTTCAG |
| | GGAAAAT (SEQ ID NO: 22) |

The above describe strategy for HR benefits from internal verification, because the targeting vector does not contain an independent promoter region, and, therefore, it is not expected to express mCherry unless it is correctly integrated to the predicted region in the DAZL locus. In the absence of a promoter, only upon correct integration to one of the alleles, but not both of them, is mCherry mRNA transcribed and the protein expressed. Because DAZL is essential for the survival of PGCs, and knocking-in mCherry to the DAZL allele renders the endogenous gene inactive, if both alleles undergo HR, there is no DAZL gene activity, and PGCs die.

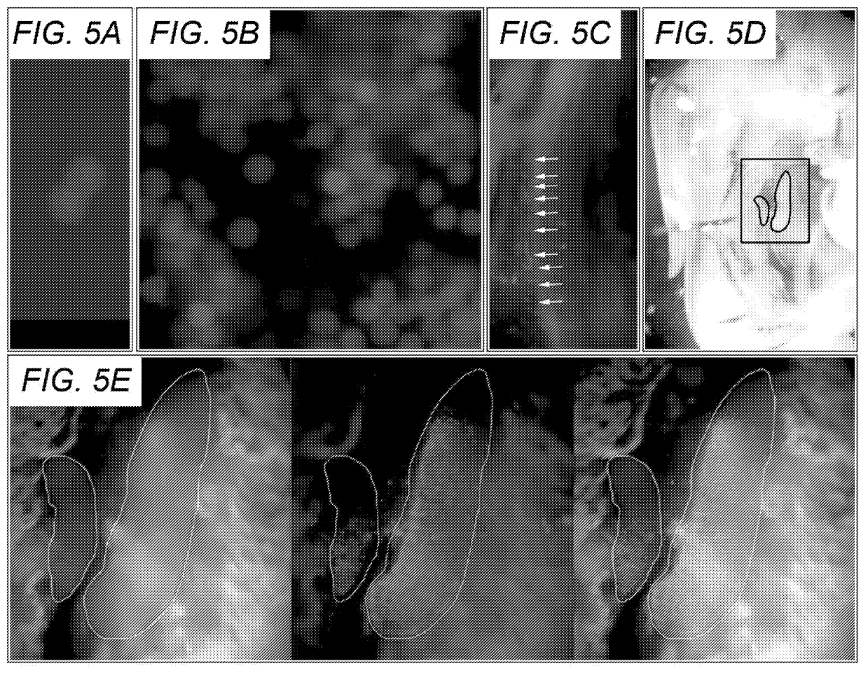

As an overview, here, FIG. 5A shows a pool of cultured PGCs following transfection with two positive cells. FIG. 5B shows a pure colony which originated from the pool of cultured PGCs shown in FIG. 5A. FIG. 5C shows successful migration of the cells of FIG. 5B to the genital anlage, and FIGS. 5D-5E show incorporation of the cells of FIG. 5B into the ovary.

Following co-electroporation of PGCs with the targeting vector and the px3361-DAZL-CRISPR1 plasmids, the cells were allowed to recover, and 48 hours later, viable dividing mCherry positive cells were observed in the culture (FIG. 5A). mCherry positive cells were collected using FACS and grown to form stable colonies (FIG. 5B). Genomic DNA from these colonies was analyzed to further confirm the homologous recombination as described above (FIG. 4C).

Generating Surrogate Chimera Embryos

To create a surrogate chimera embryo, modified PGCs are injected to the blood stream of a Hamburger-Hamilton (HH) stage 14-16 embryo, at the time when endogenous PGCs migrate to and colonize the genital ridge. Thus, in addition to the transplanted PGCs, the surrogate embryo has its own endogenous unmodified PGCs. Both PGC populations colonize the embryonic gonad and upon sexual maturity give rise to gametes. To increase the chances that a gamete is formed from the modified PGCs, a treatment to reduce the amount of endogenous PGCs can be carried out. Elimination of endogenous PGCs can be referred as “sterilization”. This can be achieved by chemical treatment using, e.g., 1,4-butanediol dimethanesulfonate (BUSULFAN™; SIGMA-ALDRICH™), or by gamma or X-ray irradiation, using, e.g., the BIOBEAM GM™ gamma irradiator (OMNIA HEALTH™, USA) prior to incubation. In certain embodiments, partial elimination of endogenous PGCs may be referred to as “sterilization”.

Administration of the primordial germ cells to the recipient animal in ovo can be carried out at any suitable time at which the PGCs can still migrate to the developing gonads. In one embodiment, administration is carried out from about stage IX according to the Eyal-Giladi & Kochav (EGK) staging system to about stage 30 according to the Hamburger-Hamilton (HH, H&H; Hamburger & Hamilton (1951) J. Morphol. 88(1): 49-92) staging system of embryonic development, and in another embodiment, at stage 15 HH. For chickens, the time of administration is, therefore, during days 1, 2, 3, or 4 of embryonic development (e.g., day 2 to day 2.5). Administration is typically by injection into any suitable target site, such as the region defined by the amnion (including the embryo), the yolk sac, etc. In one example, injection is into the embryo itself (including the embryo body wall). Alternatively, intravascular or intracoelomic injection into the embryo is employed, or the injection is performed into the heart. The methods of the presently disclosed subject matter can be carried out with prior sterilization of the recipient bird in-ovo or ex-ovo. As described above, “sterilized” embryos are partially or completely incapable of producing gametes derived from endogenous PGCs.

Specifically, freshly laid eggs were gamma irradiated at 500-700 rad using the BIOBEAM GM™ gamma irradiator (Omnia Health™, USA) and incubated with the pointed end up for 58-70 hours at 37.8° C. with 55% humidity. Following incubation, a 4-8 mm window was opened in the eggshell and 3000-8000 PGCs, which underwent successful integration of mCherry to a single DAZL locus (FIG. 5B), were injected to the blood stream, through the heart, using a sharpened micropipette having a bore of −30-40 μm. Following injections, the window was covered with white egg membrane and further sealed with a Parafilm (Parafilm) or a Leukoplast (BSN medical GmbH) tape. Embryos were incubated for several days, to confirm PGCs migration to the embryonic gonads, or until hatching with the blunt end facing up, with 45 degrees rotation every 30 min, at 37.8° C. with 55% humidity. Selected injected embryos were isolated 48 hours following injection by extraction from the eggs, washed in phosphate buffered saline (PBS), and mounted on a silicon-coated plate to be analyzed under fluorescent microscope as shown in FIG. 5C. In these embryos, the mCherry positive PGCs were found to be located in the anlage of the embryonic gonads, in the genital ridge (FIG. 5C, arrows). Additional embryos were allowed to develop for 8 days following PGCs injection and were dissected, ventral-side-up, to observe the gonads as shown in FIG. 5D, where the outlines of the gonads are delineated with a red line. FIG. 5E shows higher magnification of the inset region denoted by a blue rectangle in FIG. 5D. FIG. 5E, taken under a fluorescent lighting source, compares fluorescence on the green channel as a negative control (left panel) with fluorescence on the red channel (middle panel) and an overlapping merged image of both channels (right panel). FIG. 5E shows female phenotype gonads (delineated with white lines) which numerous mCherry positive cells colonized (red dots, middle panel), confirming that the injected PGCs are capable of migrating from the bloodstream and entering the gonads. (Typically, the PGCs migrate to the gonads at approximately days 2-3 of incubation. At this stage, the two gonads have no sexual identity, yet. Only after 8-9 days of incubation, in female embryos, the left gonad becomes an ovary and the right gonad, into which some of the PGCs had previously migrated, regresses.)

Rearing Surrogate Chimera Chicks to Sexual Maturity and Screening for Founder Chicks

PGCs-injected surrogate embryos are placed in the incubator until the chicks hatch. The chicks are then reared to sexual maturity under standard rearing conditions, e.g., according to the management guide of the breeding companies, such as Hy-Line® or Lohmann™ (see, e.g., Hy-Line® Management Guide—Brown Layers, Hy-Line® International BRN.COM.ENG November 2018, rev 9-4-19, https://www.hyline.com/filesimages/Hy-Line-Products/Hy-Line-Product-PDFs/Brown/BRN%20COM%20ENG.pdf). Typically, layer-type chickens will reach to sexual maturity at 18 weeks of age and broiler at 28 weeks of age. At this stage, sperm taken from male surrogate roosters can be analyzed using PCR (e.g., using primers P1 and P2), to determine the ratio between sperm cells derived from the modified PGCs and from the endogenous PGCs. A sperm sample which shows a ratio great than 1% (>1%) of the modified PGCs can be used to inseminate hens, and the chicks which hatch from this cross are screened by PCR to identify founders. Likewise, surrogate hens can be inseminated, and the resulting chicks are then screened (e.g., using primers P1 and P2) to identify founders. Likewise, surrogate hens can be inseminated, and the resulting chicks are then screened to identify founders. The founders are expected to be heterozygotes and otherwise healthy and fertile. Crossing between two heterozygotes will yield a Mendelian segregation ratio of 1:4 homozygote sterile embryo, in which the two alleles of DAZL are replaced by mCherry. Further propagating the flock into future generations and maintaining the trait can be accomplished by maintaining the trait using heterozygous individuals.

### Example 18: Layer Hens Lay Eggs with Broiler Embryos

Objective: The objective here was to transfer foreign genomic material obtained from a broiler chicken to an embryo of a surrogate layer hen, and thereby produce a next generation broiler chicken.

Methods: Methods used here are based on the transfer of foreign genomic material to a different breed of chicken; in this case the transfer of foreign genomic material from a broiler chicken to a layer hen. This transfer was performed by injecting genomic material from the broiler into a surrogate embryo of the layer hen, wherein the next generation of chicks hatched included broilers and broiler/layer chicks. Methods are described, in brief below.

The genomic material originated from primordial germ cells (PGCs) collected from a broiler chicken, which were then injected into the surrogate embryos where they colonize the gonad of the surrogate layer hen and upon sexual maturity, the gonads give rise to gametes, which contain the genomic material originating from the injected PGCs.

If the injected surrogate embryo has no endogenous PGCs— in other words it is sterile, then all of the gametes would be derived from the injected PGCs. That was not the case here as the gamma-irradiation used to eliminate endogenous PGCs in the surrogate embryos is not 100% effective. If the injected surrogate has endogenous PGCs, which was the case here, then the gametes will be mixed.

Gamma-irradiation of the surrogate embryo was employed to reduce the amount of endogenous PGCs present in the embryo, thus giving a better chance to the exogenous PGCs from the broiler to give rise to mature gametes. The results presented here demonstrate the conceptual feasibility of transferring foreign genomic material, in this case using layer breed hens to lay broiler chicks.

WT broiler PGCs were collected from female broiler embryo, and cultured in-vitro. These PGCs were injected into the bloodstream of gamma-irradiated (780 Rad) surrogate female layer-breed embryo. Following injection, the embryo was incubated and hatched under normal condition. Upon reaching to sexual maturity, the hen was artificially inseminated with broiler sperm. The fertile laid eggs were incubated until the hatch of the chicks.

Results:

The chicks were reared until the age of 7 week. From the cross described in the methods above, the expected genotypes of the offspring are divided into two: (1) chicks that are half broiler and half layer. This would be the case if the broiler sperm fertilized an endogenous layer egg; or (2) chicks that genetically are only broiler. In this case, broiler sperm fertilization would have been of a broiler-injected PGCs-derived egg.

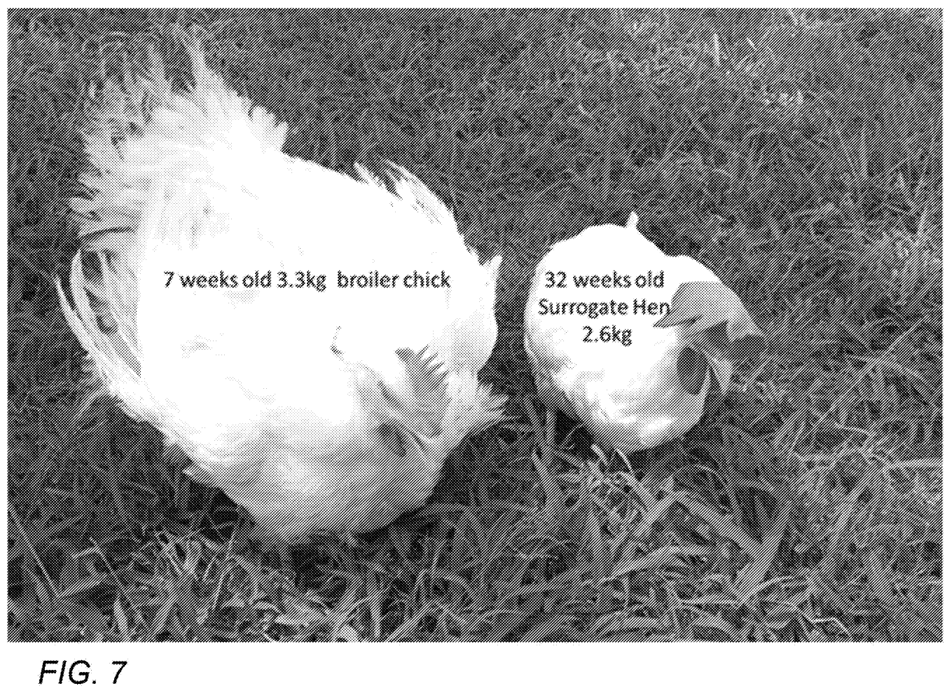

The genotypes of the two possible chicks differ greatly, which results in significantly different chick morphologies due to different growth rates. Therefore, it was possible to distinguish between the two possible chick outcomes by monitoring the body weight of the hatched chicks over time. This was a challenging experiment since the endogenous eggs tend to have better chance to ovulate than the exogenous injected PGCs-derived eggs. Yet, a broiler male chick was identified in this experiment, as shown in FIG. 7.

FIG. 7 presents a photograph of the layer-breed hen that laid the egg from which the broiler-type chick hatched. Broiler-derived PGCs were injected to a gamma-irradiated layer-type female embryo, which was further incubated, hatched, and reached sexual maturity. The hen was inseminated with WT broiler sperm. The surrogate hen, shown on the right, reached to a weight of 2.6 kg at the age of 32 weeks. At about 22 weeks of age, this hen laid an egg that was incubated for 3 weeks, and the hatch male chick was reared and reached to a weight of 3.3 kg, by the age of 7 weeks, as shown on the left.

To validate that the broiler male chick originated from an egg that was derived from the injected PGCs, a genetic motherhood test was performed. In this test, genomic DNA samples were taken from the injected PGCs, the surrogate hen (FIG. 7 on the right), a male sibling chick that was half broiler (from the sperm) and half layer (the surrogate hen), and from the broiler chick (FIG. 7 on the left). These 4 genomic samples were used as templates for PCR to identify differentially presented Single Nucleotides Polymorphism nucleotides (SNPs).

A 925 nucleic acid nucleotide sequence, corresponding to GRCg6a:Z:9033281:9034205:1 genomic region (SEQ ID NO: 54), containing several annotated SNPs was PCR amplified from all 4 samples using the FWD primer CTCCTACCTGCCTCTTCTTC (SEQ ID NO: 55) and the reverse primer TCTTCTCTGCCCATTAGAGC (SEQ ID NO: 56), and the PCR products were sequenced and screened in regions containing known annotated SNPs.

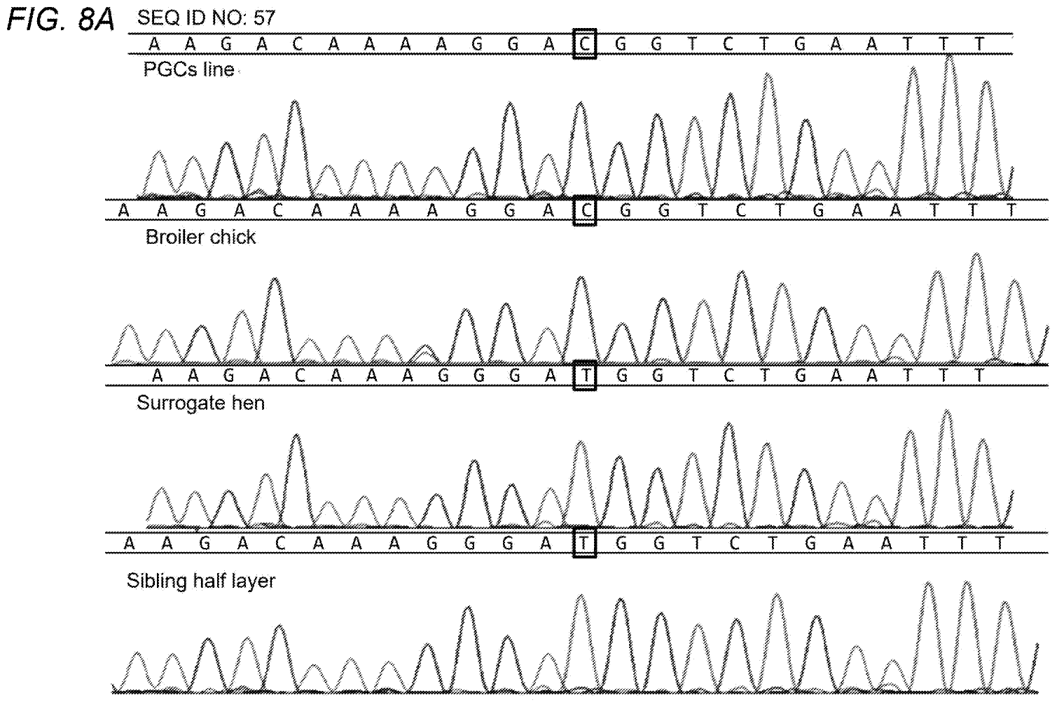

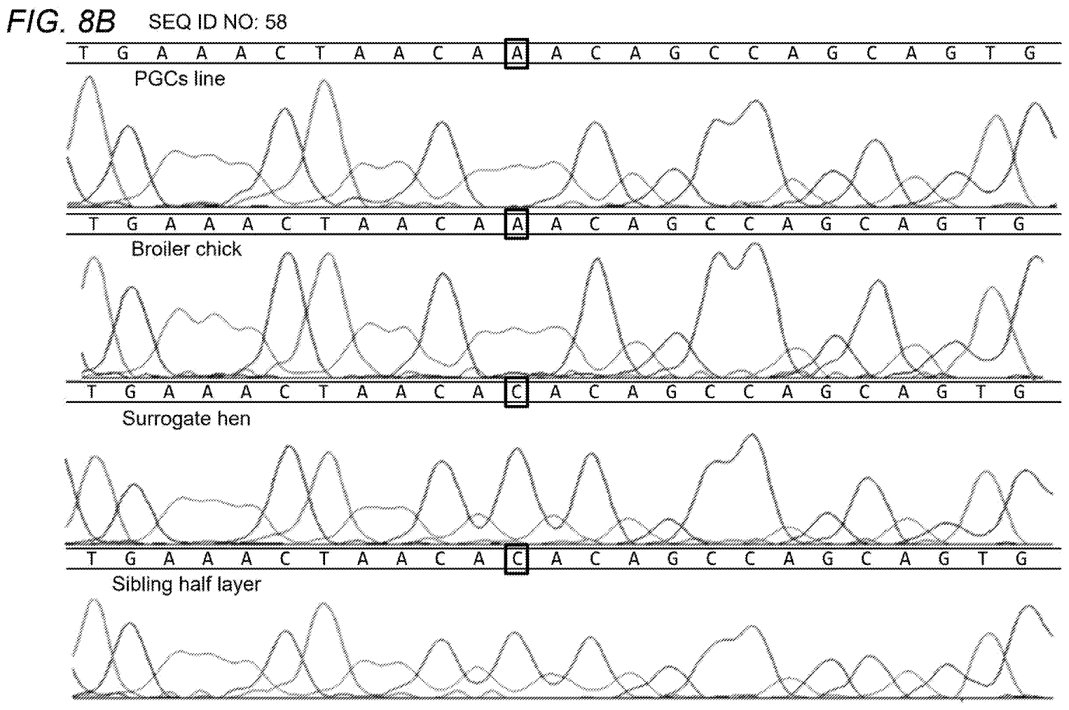

Two SNPs were found to differentiate between the genome of the broiler chick and the surrogate hen. The first SNP (dbSNP:rs736292769 C/T) was found to be C in the PGCs and the broiler chick, while the surrogate hen and the sibling, which is half layer was T (FIG. 8A). The second SNP (dbSNP:rs731066568 C/A) was found to be A in the PGCs and the broiler chick, while the surrogate hen and the sibling, which is half layer was C (FIG. 8B). The results of this motherhood test indicate that the broiler chick was originated form an egg that was derived from the injected PGCs.

Summary: Despite the challenges, it is shown here that a genetically distinct Broiler male chick was hatched from a layer hen.

### Example 19: Knocking Out the DEAD-Box Helicase 4 (DDX4, AKA Chicken VASA Homologue—cVH) Gene in PGCs

Objective: The objective here was to knockout a gene of interest (GOI) in PGCs that could then be used to transfer foreign genomic material to a surrogate embryo.

Methods: Highly conserved from nematodes to humans, and well annotated in the literature, the RNA binding protein DDX4 (DEAD-Box Helicase 4), which is on the Z chromosome in chicken NC 052572.1 GRCg7b Z:17471862 . . . 17503681) is an essential genes for gametogenesis. In accordance with its role, its expression is restricted to PGCs in all organisms including chicken. Thus it serves as a germ-cell lineage marker.

Numerous model-organisms, including c. elegans, Drosophila fly, fish, frog, and mice, demonstrate that inactivity of DDX4, results in sterility due to failure in gamete formation. Located on the Z sex chromosome, chicken DDX4-null sterile female hens can be obtained, by crossing WT female hens with males, which are heterozygotes for a null-mutation in this gene. Clearly, by definition sterile chickens cannot breed, therefore fertile heterozygotes colonies that carry the mutated allele are needed to obtain DDX4—null embryos. These can be generated from DDX4 heterozygotes male PGCs.

To generate heterozygotes PGCs harboring a null-mutation on one allele of DDX4, only male-derived PGCs can be used, as they have two Z chromosomes. Thus, male-derived PGCs were electroporated with RNP CRISPR-Cas9 with two sgRNA sequences. The first GCTGGCATTCGCTATGGAGG GRCg6a:Z:16929641:16929660:1 (SEQ ID NO: 59) is located at the 5′ end of the second exon, with predicted cleavage site at the ATG codon of the first Methionine amino acid. The second sgRNA TTCTGAGGAGCAGGCGTGGA GRCg6a:Z:16929713:16929732:1 (SEQ ID NO: 60) is located at the 3′ end of the second exon.

Results:

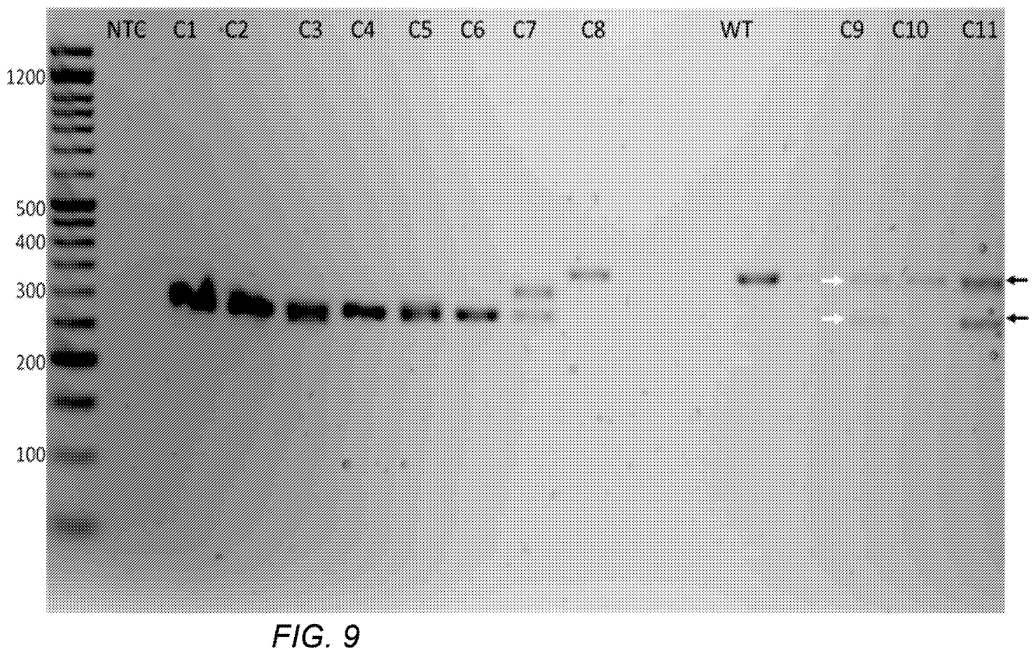

The predicted deletion between the two breaking points is about 70 bp, which forms a null mutation at the beginning of the coding region of DDX4. Following electroporation and introduction of the RNP complexes, the cells were allowed to recover for 72 hours, and sorted by FACS for collection as single cells in a 96 well plate, with one cell per well. Each cell gave rise and formed a pure colony, and genomic DNA from these colonies was analyzed using PCR amplification, as shown in FIG. 9. For this PCR, the FWD primer that was used is: AATGGAGCCATAGCAGAGCC (SEQ ID NO: 61) and the REV primer is: TGCAGGAAGCACTGCGAAG-62 (SEQ ID NO: 62). These primers flank the predicted deletion site. On the WT allele the predicted product size is 343 bp, and on the truncated allele the predicted size is 273 bp.

FIG. 9 presents the results of a PCR analysis to identify the genotype of pure colonies. Genomic DNA from 11 colonies (C1-C11) was extracted, and used as template for PCR with the primers described above, SEQ ID NO: 61 (Fw) and SEQ ID NO: 62 (Rev). Colonies C1-C6 show a single band with the predicted size of only the truncated allele. Colony C7 shows two products, which are shorter than the WT allele. Colonies C8 and C10 show a single product of a similar length of the WT allele. Colonies C9 and C11 show two products, the upper band at the size of the WT allele and the lower band at the predicted size of the truncated allele (arrows). Thus, C9 and C11 are expected to carry the desired heterozygotes mutation. To validate this, genomic DNA from colony C11 was taken for sequencing.

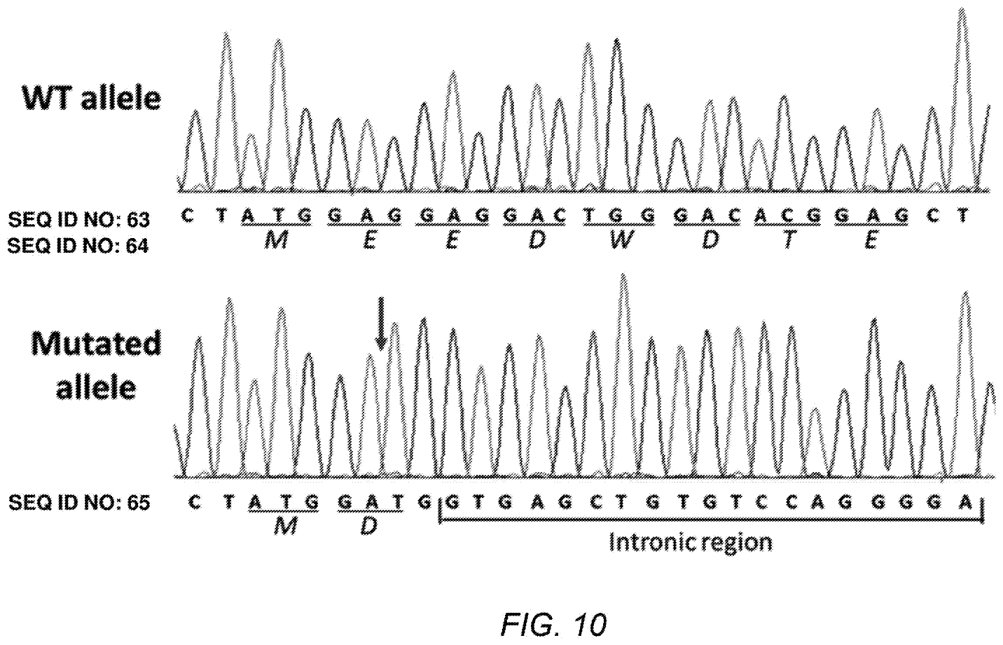

The alleles of colony C11 were sequenced individually, as shown in FIG. 10, confirming that one allele is WT and the second allele harbors a deleterious 70 bp deletion which renders the DDX4 copy on this allele null. The part of the sequence shown in FIG. 10 corresponds to the beginning of the coding region of the DDX4 gene. The coding codons are underlined and the corresponding amino acids are written in italics. The WT nucleotide sequence of the WT allele is CTATGGAGGAGGACTGGGACACGGAGCT (SEQ ID NO: 63), which encodes the WT amino acid start of the polypeptide: MEEDWDTE (SEQ ID NO: 64). The sequence of the nucleotide sequence of the mutated allele is: CTATGGATGGTGAGCTGTGTCCAGGGGA (SEQ ID NO: 65). The breaking point of the 70 bp deletion is marked with an arrow. The translation of this mutant allele begins with the first methionine (M), which is followed by a frame shift that leads to an introduction of a nonsynonymous amino acid—Aspartic acid (D) (See, SEQ ID NO: 64). The beginning of the intronic region that follows this exon is marked.

The sequencing confirmed that on the mutated allele the copy of the DDX4 gene is null.

Summary: Colony C11 was confirmed to be heterozygotes at the DDX4 gene locus, containing one WT allele and one null allele of DDX4. Thus, these PGCs are suitable to be used to generate chickens heterozygotes for DDX4.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A gene-edited or genetically modified avian primordial germ cell (PGC) comprising a first genetic modification on a chromosome, said modification modifying a trait in the PGC or in the gene-edited or genetically modified avian produced by the PGC or a combination thereof, when compared to an isogenic PGC or isogenic avian lacking the first genetic modification, the modified trait inducing sterility in the gene-edited or genetically modified avian produced by the PGC without impairing viability of the gene-edited or genetically modified avian produced by the PGC.

2. The gene-edited or genetically modified avian PGC of claim 1, wherein the first genetic modification comprises modification of a gene having an isolated function specific to a PGC or modification of a gene having a function specific to gametogenesis, gamete maturation, or gamete function, wherein said specific PGC functional modification reduces or inhibits survival, maturation, or differentiation of a PGC derived from the gene-edited or genetically modified avian or said specific gametogenesis, gamete maturation, or gamete functional modification reduces or inhibits gametogenesis, meiosis, gamete function, or gamete fertilization in the gene-edited or genetically modified avian.

3. The gene-edited or genetically modified avian PGC of claim 1, wherein the first genetic modification comprises modification of a gene encoding at least one protein selected from a DEAD-Box Helicase 4 (DDX4) protein, a Deleted In Azoospermia-Like (DAZL) protein, a Zona Pellucida Binding Protein 1/2 (ZPBP1/2) protein, a Cyclin-Dependent Kinases Regulatory Subunit 2 (CKS2) protein, a Spermatogenesis Associated 16 (SPATA16) protein, a Serine/Threonine-Protein Phosphatase PP1-Gamma Catalytic Subunit (PPP1CC) protein, an Izumo Sperm-Egg Fusion 1 (IZUMO1) protein, a Synaptonemal Complex Central Element Protein 1 (SYCE1) protein, a YTH Domain-Containing 2 (YTHDC2) protein, a Meiosis Specific With Coiled-Coil Domain (MEIOC) protein, a Septin-4 (SEPT4) protein, a Stromal Antigen 3 (STAG3) protein, a Nanos C2HC-Type Zinc Finger 3 (NANOS3) protein, a Deleted In Azoospermia 1 (DAZ1) protein, or a combination thereof.

4. The gene-edited or genetically modified avian PGC of claim 1, where said first genetic modification comprises modification of at least 2 genes.

5. The gene-edited or genetically modified avian PGC of claim 1, wherein the modified trait induces sterility in a male gene-edited or genetically modified avian produced by the PGC and a female gene-edited or genetically modified avian produced by the PGC.

6. The gene-edited or genetically modified avian PGC of claim 1, further comprising a second genetic modification on the same chromosome as the first genetic modification, the second genetic modification encoding a marker detectable in the PGC, in the gene-edited or genetically modified avian produced by the PGC, or in a PGC produced by the gene-edited or genetically modified avian produced by the PGC, and wherein the marker is a fluorescent protein, a luminescent protein, or a chromoprotein detectable in the cytoplasm of the PGC.

7. The gene-edited or genetically modified avian PGC of claim 1, wherein the gene-edited or genetically modified avian PGC is derived from an avian of the Galliformes order, the Anseriformes order, the Otidiformes order, the Columbiformes order, or the Struthioniformes order.

8. The gene-edited or genetically modified avian PGC of claim 7, wherein when derived from the Galliformes order, the gene-edited or genetically modified avian PGC is derived from an avian of the Numidia family or the Phasianidae family comprising the Gallus genus or the Meleagris genus.

9. A gene-edited or genetically modified avian embryo comprising gene-edited or genetically modified avian primordial germ cells (PGCs), each gene-edited or genetically modified avian PGC comprising a first genetic modification modifying a trait in the gene-edited or genetically modified avian embryo and/or in the PGC produced by the gene-edited or genetically modified avian embryo as an adult when compared to an isogenic avian embryo or a PGC produced by an isogenic avian embryo as an adult lacking the first genetic modification, the modified trait inducing sterility in the gene-edited or genetically modified avian embryo, as an adult without impairing viability, or in the gene-edited or genetically modified avian offspring produced by the PGC produced by the gene-edited or genetically modified avian embryo as an adult without impairing viability of the gene-edited or genetically modified avian offspring produced by the PGC.

10. A gene-edited or genetically modified avian comprising gene-edited or genetically modified avian primordial germ cells (PGCs), each gene-edited or genetically modified avian PGC comprising a first genetic modification modifying a trait in the gene-edited or genetically modified avian and/or in the PGC produced by the gene-edited or genetically modified avian when compared to an isogenic avian or a PGC produced by an isogenic avian lacking the first genetic modification, the modified trait inducing sterility in the gene-edited or genetically modified avian without impairing viability, or in a gene-edited or genetically modified avian offspring, without impairing viability of the gene-edited or genetically modified avian offspring produced by the PGC.

11. A deoxyribonucleic acid (DNA) editing system comprising:
(a) a first agent comprising a first nucleic acid sequence wherein
(i) the first nucleic acid sequence comprises a mutated or null gene of interest (GOI) sequence or a fragment thereof, the mutated or null GOI comprising
an isolated function specific to a PGC, or
a function specific to gametogenesis, gamete maturation, or gamete function in the gene-edited or genetically modified avian;
(ii) the first nucleic acid sequence comprises or encodes an endonuclease enzyme that can carry out genome editing; or
(iii) insertion of the first nucleic acid sequence in a chromosome of interest modifies or disrupts the targeted GOI, the targeted GOI having:
an isolated function specific to a PGC; or
a function specific to gametogenesis, gamete maturation, or gamete function in the gene-edited or genetically modified avian; and
(b) a second agent comprising a second nucleic acid sequence, the second nucleic acid sequence encoding a recombinase enzyme and a sequence for directing the second nucleic acid sequence to the targeted GOI on the chromosome of interest of the PGC.

12. The DNA editing system of claim 11, wherein the modification or disruption of a gene having a function specific to gametogenesis, game maturation, or gamete function reduces or inhibits gametogenesis, meiosis, gamete function, or gamete fertilization in the gene-edited or genetically modified avian.

13. The DNA editing system of claim 12, the modification or disruption of a gene comprising modification or disruption of a gene encoding a protein selected from the group consisting of DEAD-Box Helicase 4 (DDX4) protein, Deleted In Azoospermia-Like (DAZL) protein, Zona Pellucida Binding Protein 1/2 (ZPBP1/2) protein, Cyclin-Dependent Kinases Regulatory Subunit 2 (CKS2) protein, Spermatogenesis Associated 16 (SPATA16) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis-Specific With Coiled-Coil Domain-Containing Protein (MEIOC) protein, Septin-4 (SEPT4) protein, Stromal Antigen 3 (STAG3) protein, Nanos C2HC-Type Zinc Finger 3 (NANOS3) protein, and Deleted In Azoospermia 1 (DAZ1) protein, or a combination thereof.

14. The DNA editing system of claim 11, wherein the sequence for directing the first nucleic acid sequence or the second nucleic acid sequence to the chromosome of interest of the PGC comprises:
(a) a left homology arm (LHA) nucleotide sequence that is substantially homologous to the 5′ region flanking a target gene locus in the chromosome of interest of the PGC; and
(b) a right homology arm (RHA) nucleotide sequence that is substantially homologous to the 3′ region flanking the target gene locus in the chromosome of interest of the PGC.

15. The DNA editing system of claim 11, wherein the first nucleic acid sequence or the second nucleic acid sequence comprises a detectable marker.

16. The DNA editing system of claim 11, wherein:
(a) the first nucleic acid sequence comprises any one of the sequences set forth in SEQ ID NO: 10, SEQ ID NO: 33, or SEQ ID NO: 34; and
(b) the second nucleic acid sequence comprises sequences set forth in SEQ ID NO: 37.

17. A method for producing a gene-edited or genetically modified avian, the method comprising:
(a) obtaining a primordial germ cell (PGC) from an avian;
(b) stably integrating into at least one targeted gene of interest (GOI) on a chromosome of interest in the PGC
(i) a first exogenous polynucleotide operatively linked to a recombinase recognition site, the first exogenous polynucleotide comprising a mutated or null GOI sequence or fragment thereof or encodes an endonuclease enzyme that can carry out genome editing, the first exogenous polynucleotide eliciting a sterility-inducing phenotype in the PGC or in a gene-edited or genetically modified avian derived from the PGC, and wherein insertion of the first exogenous polynucleotide modifies or disrupts the targeted GOI, the targeted GOI having
an isolated function specific to a PGC; or
a function specific to gametogenesis, gamete maturation, or gamete function in the gene-edited or genetically modified avian; and
(ii) a second exogenous polynucleotide encoding a recombinase enzyme;
(c) producing pure PGC colonies that comprise the first exogenous polynucleotide and the second exogenous polynucleotide;
(d) transplanting a pure PGC colony into a male chick embryo to produce a chimera male chick embryo and transplanting a pure PGC colony to a female chick embryo to produce a chimera female chick embryo;
(e) producing a chimeric founder adult avian by hatching and rearing the chimera founder chicks to sexual maturity;
(f) screening the chimera founder adult avian to verify heterozygosity for the edited GOI;
(g) breeding a male chimera founder adult avian having heterozygosity for the edited GOI with a female chimera founder adult avian having heterozygosity for the edited GOI to produce progeny embryos;
(h) identifying a homozygotic embryo from the progeny embryos; and
(i) hatching and rearing the avian;
thereby producing the avian.

18. The method of claim 17, wherein the at least one targeted GOI comprises a gene encoding a protein selected from the group consisting of DEAD-Box Helicase 4 (DDX4) protein, Deleted In Azoospermia-Like (DAZL) protein, Zona Pellucida Binding Protein 1/2 (ZPBP1/2) protein, Cytokine-Dependent Kinases Regulatory Subunit 2 (CKS2) protein, Spermatogenesis Associated 16 (SPATA16) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis-Specific With Coiled-Coil Domain-Containing Protein (MEIOC) protein, Septin-4 (SEPT4) protein, Stromal Antigen 3 (STAG3) protein, Nanos C2HC-Type Zinc Finger 3 (NANOS3) protein, and Deleted In Azoospermia 1 (DAZ1) protein, or any combination thereof.

19. The method of claim 17, wherein the sequence for directing the first nucleic acid sequence or the second nucleic acid sequence to the chromosome of interest of the PGC comprises:
(a) a left homology arm (LHA) nucleotide sequence that is substantially homologous to the 5′ region flanking a target gene locus for the targeted GOI in the chromosome of interest of the PGC; and
(b) a right homology arm (RHA) nucleotide sequence that is substantially homologous to the 3′ region flanking the target gene locus for the targeted GOI in the chromosome of interest of the PGC.

20. The method of claim 17, wherein:
(a) the first exogenous polynucleotide comprises any one of the sequences set forth in SEQ ID NO: 10, SEQ ID NO: 33, or SEQ ID NO: 34; and
(b) the second exogenous polynucleotide comprises sequences set forth in SEQ ID NO: 37.

21. The method of claim 17, the PGC derived from an avian of the Galliformes order, the Anseriformes order, the Otidiformes order, the Columbiformes order, or the Struthioniformes order.
